(19) United States
(12) Patent Application Publication
(10) Pub. No.: US 2025/0332115 A1
(43) Pub. Date: Oct. 30, 2025

(54) CONTROLLED EXPRESSION OF THERAPEUTICALLY RELEVANT BIOMOLECULES FOR LUMEN-LOCALIZED PAYLOADS IN BIOMIMETIC NANOVESICLES AND EXOSOMES

(71) Applicant: Thomas MALCOLM, Andover, NJ (US)

(72) Inventor: Thomas MALCOLM, Andover, NJ (US)

(21) Appl. No.: 18/868,294

(22) PCT Filed: May 25, 2023
(86) PCT No.: PCT/US2023/023532

§ 371 Date: Nov. 22, 2024

Related U.S. Application Data
(60) Provisional application No. 63/345,659, filed on May 25, 2022.
(60) Provisional application No. 63/424,978, filed on Nov. 14, 2022.

(51) Int. Cl. A61K 9/50; A61K 38/17; A61K 38/48; A61K 40/11; A61K 40/31; A61K 40/42; C12N 5/0783

(52) U.S. Cl. CPC A61K 9/5068; A61K 38/1709; A61K 38/482; A61K 40/11; A61K 40/31; A61K 40/421; C12N 5/0636; C12N 5/0646; A61K 2239/39

(57) ABSTRACT

Disclosed herein are methods of generating therapeutic biomimetic nanovesicles (BioNVs) or therapeutic exosomes with lumen-loaded, therapeutically relevant biomolecules from hypoimmunogenic cells, compositions of therapeutic BioNVs or therapeutic exosomes, and methods of using the same for treatment or prevention of a disease or disorder.

### CROSS-REFERENCE TO RELATED APPLICATIONS

This application claims the benefit of and priority to U.S. Provisional Application No. 63/345,659, filed May 25, 2022, and U.S. Provisional Application No. 63/424,978, filed Nov. 14, 2022, the contents of which are herein incorporated by reference in their entirety.

### TECHNICAL FIELD

The present disclosure provides, in part, methods of preparing lumen-loaded biomimetic nanovesicles and exosomes and compositions of and methods of using the same for the treatment of disease, e.g., in a mammalian subject, such as a human.

### BACKGROUND

Cell-derived vesicles (CDVs), including biomimetic nanovesicles (BioNVs) and exosomes, are a powerful and novel tool for the treatment of a multitude of diseases including cancers, hereditary conditions, tissue damage, endocrine conditions, and infectious diseases. BioNVs can be derived from cells through various cell disruption processes. By contrast, exosomes are naturally shed from whole cells via endocytic pathways. The diameter and volume (the latter, defined by the lumen compartment within the lipid bilayer) of both BioNVs and exosomes can be pre-determined depending on the methods used for disrupting the cells (or treating the cells for exosomes) from which they are derived.

The lumen of BioNVs can be loaded with therapeutically relevant molecules by three main methods: (1) the spontaneous inclusion of biomolecules (nucleic acids, proteins/peptides) into the lumen of the CDVs that have been expressed from within the cells from which the CDVs are derived; (2) the addition of (bio)molecules (nucleic acids, proteins/peptides, small molecules, or biologics) into segmented concentration chambers during BioNV formation ensuring controlled loading; or (3) a combination of (1) and (2), where essential cell-derived biomolecules are packaged in the lumen of the CDV while the desired biomolecules are added for specific purposes, for example, the addition of doxorubicin for delivery to a targeted cancer cell.

Cells express a multitude of biomolecules (cytokines, chemokines, regulatory nucleic acids, etc.) that aid in cellular regeneration, tissue repair, and cytotoxicity through complex pathways that are generally governed by external stimuli. These biomolecules are tightly regulated through cellular pathways that typically begin with surface receptors that sense environmental stimuli, followed by the triggering of intracellular domains of the receptors, that result in the intracellular propagation of signal transduction events that can involve complex cross-talk between signaling pathways. The endpoints of these signaling pathways is generally a system of transcriptional activators and repressors that regulate gene function and expression of the biomolecules. Layered within these complex and orchestrated pathways are numerous types of transitioning effector molecules and biomolecules such as phosphate containing compounds (cAMP, ATP, GTP, etc.), metallic ions (calcium, iron, zinc, etc.), signal modulating enzymes (phosphatases, kinases, phosphorylases, etc.), transcription factors and initiators, and translation factors, to name a few.

Each type of cell within an organism has a battery of different surface receptors, each with distinct downstream functionality that ultimately leads to a specific biomolecule repertoire. The expression of these biomolecules from healthy cells generally contains effector functions designed to protect and maintain homeostasis the cell, or the tissue within which they are located, and the overall survival of the organism. For example, T cells contain T-cell Receptors (TCRs) or Chimeric Antigen Receptors (if engineered), that when engaged with a pathogen infected or transformed cell, trigger the T cell to initiate the rapid expression of inflammatory and apoptotic cytokines including interferons, perforins, granzymes, and granulysins that form lytic granules in the cytoplasm that localize subsequently to the sight of the TCR and target cell ligand interface, called the Immunological Synapse (IS). In another example, M1 macrophages are activated through the CD38 surface receptor causing the cells to resist bacterial invasion and activate the phagocytosis and digestion of necrotic cells via pro-inflammatory cytokines. Also, M2 macrophages are activated through the EGR2 surface receptor causing the cells to enter repair mode by producing (in part) anti-inflammatory cytokines. In another example, Natural Killer cells contain multiple surface and transmembrane receptors linked to complex intercellular signaling networks that give the cell exceptional environmental sensory properties so that the cell recognizes the difference between healthy cells and infected or transformed cells for destruction, with high precision. This complex network is so sensitive that Natural Killer cells have adapted specific receptors that are coordinated with multiple intercellular signaling pathways, often in a synergistic manner, to regulate its cytotoxic phenotype for specific diseases caused by viral or bacterial infection, genetic abnormalities, or cancers. This level of synergy among receptor-signaling pathways is necessary for Natural Killer cells as they are primed/pre-loaded with cytotoxic secretory lysosomes containing perforins. granzymes, granulysin and other cytotoxins that could wreak havoc if they are not tightly controlled. CD4+ and some subsets of CD8+ T-cells (with the exception of CD8+ Killer T-cells that are primed with low levels of lytic granules) are not primed with lytic granules, and thus do not require as tight of a biochemical regulation network.

In addition to immunomodulatory cells, recent reports have shown that there is a strong correlation of cytokine signaling between leukocytes and cardiomyocytes, and that the interaction plays an important role in controlling inflammation after cardiac injury. The pro-inflammatory cytokines tumor necrosis factor TNF-α, interleukin-6 (IL-6), and IL-1β, have been observed in elevated levels in patients with cardiac injury. These cytokines can be triggered under pathologic conditions in cardiac tissue, and a runaway pro-inflammatory effect has been linked in patients to high mortality cases of heart failure. In cardiomyocytes, the expression of IL-6 at low levels can have repairing benefits to heart tissue, but at high levels can lead to runaway inflammation and heart failure. IL-6 is regulated through the levels IL-1β, suggesting that the direct regulation of IL-1β can be manipulated to tune the levels of IL-6 in a positive and controlled manner.

The packaging of desired biomolecules that have therapeutic value into delivery systems such as AAVs, Anelloviridae, or lipid nanoparticles (LNPs), requires complicated packaging processes. Currently, the concentration of biomolecule(s) and variety of biomolecule(s) to can be packaged in these systems is very limited and dependent on engineered promoter-gene constructs and packaging size limits. Further, naturally shed exosomes, although capable of combining multiple biomolecules from the cell from which they are shed, will generally have lower, less desirable, and less ‘controllable’ concentrations of the desired biomolecule(s) (compared to BioNVs) due to the canonical pathways that are committed to these types of extracellular vesicles (EVs). Thus, additional systems are required for controlled loading into the lumen. Additionally, CDVs derived from resting cells that are in a ‘natural somatic state’ are limited as therapeutic sources because the naturally occurring biomolecules with therapeutic properties have low (or absent/repressed) expression levels. As such, there remains a need for a method to control and regulate the genetic expression of therapeutically desired/relevant biomolecules within a cell's genotypic repertoire for the purpose of packaging them into the lumen of a BioNV (or exosome) such that the concentration and variety of the therapeutically desired biomolecules can make treatment of disease feasible.

### SUMMARY

In embodiments, a method of generating a therapeutic biomimetic nanovesicle (BioNV) comprising (a) obtaining a hypoimmunogenic cell, (b) activating the hypoimmunogenic cell to express one or more therapeutically relevant biomolecules, and (c) processing the activated, hypoimmunogenic cell to generate the therapeutic BioNV, wherein the therapeutic BioNV comprises the hypoimmunogenic cell plasma membrane and encapsulates the one or more therapeutically relevant biomolecules.

In embodiments, a method of generating therapeutic Extracellular Vesicles (EVs) such as exosomes and microsomes comprising (a) obtaining a hypoimmunogenic cell, (b) activating the hypoimmunogenic cell to express one or more therapeutically relevant biomolecules, (c) harvesting naturally shed therapeutic exosomes from said activated, hypoimmunogenic cell, wherein the therapeutic exosomes comprises the hypoimmunogenic cell plasma membrane and encapsulates the one or more therapeutically relevant biomolecules.

In embodiments, a method of treating or preventing a disease or disorder comprising administering a therapeutically effective amount of a therapeutic biomimetic nanovesicle (BioNV) generated by claim 1 to a subject in need thereof.

In embodiments, a method of treating or preventing a disease or disorder comprising administering a therapeutically effective amount of a therapeutic exosome generated by claim 2 in a subject in need thereof.

In embodiments, the hypoimmunogenic cell is a stem cell, an induced pluripotent stem cell (iPSC), a reprogrammed pluripotent or multipotent cell, an embryonic stem cell, a mesenchymal stem cell, or a differentiated cell which originates from any stem cell thereof. In embodiments, the hypoimmunogenic cell is a T cell, helper T cell, T-memory cell, or NK cell. In embodiments, the hypoimmunogenic cell is a macrophage. In embodiments, the hypoimmunogenic cell is a monocyte. In embodiments, the hypoimmunogenic cell is a hepatocyte, a cardiomyocyte, a neuron, an endothelial cell, a pancreatic cell, or a retinal pigmented epithelium (RPE) cell.

In embodiments, the hypoimmunogenic cell substantially lacks one or more MHC class I proteins, MHC class II proteins, T cell receptor (TCR) proteins, and/or cytokine release syndrome (CRS) proteins. In embodiments, the hypoimmunogenic cell has a β2-macroglobulin (B2M) gene disruption and/or a disruption that reduces or ablates MHC class I protein expression and/or activity. In embodiments, the hypoimmunogenic cell has a CIITA gene disruption and/or a disruption that reduces or ablates MHC class II protein expression and/or activity.

In embodiments, the hypoimmunogenic cell has an HLA-A gene disruption and/or a disruption that reduces or ablates HLA-A protein expression and/or activity. In embodiments, the hypoimmunogenic cell has an HLA-B gene disruption and/or a disruption that reduces or ablates HLA-B protein expression and/or activity. In embodiments, the hypoimmunogenic cell has an HLA-C gene disruption and/or a disruption that reduces or ablates HLA-C protein expression and/or activity. In embodiments, the hypoimmunogenic cell has an HLA-E gene disruption or an HLA-G gene disruption and/or a disruption that reduces or ablates HLA-E or HLA-G protein expression and/or activity. In embodiments, the hypoimmunogenic cell has an HLA-F gene disruption and/or a disruption that reduces or ablates HLA-F protein expression and/or activity.

In embodiments, the hypoimmunogenic cell has a T cell alpha constant (TRAC) gene disruption and/or a disruption that reduces or ablates TRAC protein expression and/or activity. In embodiments, the hypoimmunogenic cell has a T cell beta constant (TRBC) gene disruption and/or a disruption that reduces or ablates TRBC protein expression and/or activity.

In embodiments, the hypoimmunogenic cell has reduced or ablated expression of a PD-1 gene and/or reduced or ablated PD-1 protein expression and/or activity, wherein the hypoimmunogenic cell is activated; or the hypoimmunogenic cell has expression or increased expression of a PD-1 gene and/or gene product, wherein the hypoimmunogenic cell is not activated. In embodiments, the hypoimmunogenic cell has an IL-4 gene disruption and/or a disruption that reduces or ablates IL-4 protein expression and/or activity. In embodiments, the hypoimmunogenic cell has an IL-6 gene disruption and/or a disruption that reduces or ablates IL-6 protein expression and/or activity. In embodiments, the hypoimmunogenic cell has an IL-10 gene disruption and/or a disruption that reduces or ablates IL-10 protein expression and/or activity. In embodiments, the hypoimmunogenic cell has an IL-16 gene disruption and/or a disruption that reduces or ablates IL-16 protein expression and/or activity.

In embodiments, the hypoimmunogenic cell has a SerpinB9 gene disruption and/or a disruption that reduces or ablates SerpinB9 protein expression and/or activity. In embodiments, the hypoimmunogenic cell expresses or has increased expression and/or activity of a SerpinB9 gene and/or gene product.

In embodiments, the hypoimmunogenic cell expresses or has increased expression and/or activity of a CCL2 gene and/or gene product. In embodiments, the hypoimmunogenic cell expresses or has increased expression and/or activity of a PD-L1 gene and/or gene product, wherein the hypoimmunogenic cell is not activated. In embodiments, the hypoimmunogenic cell expresses or has increased expression and/or activity of a H2-M3 gene and/or gene product.

In embodiments, the hypoimmunogenic cell expresses or has increased expression and/or activity of a CD47 gene and/or gene product. In embodiments, the hypoimmunogenic cell expresses or has increased expression and/or activity of a CD24 gene and/or gene product. In embodiments, the hypoimmunogenic cell expresses or has increased expression and/or activity of a chimeric CD24/CD47 gene and/or gene product.

In embodiments, the hypoimmunogenic cell expresses or has increased expression and/or activity of a CTLA-4 gene and/or gene product. In embodiments, the hypoimmunogenic cell expresses or has increased expression and/or activity of a CD200 gene and/or gene product. In embodiments, the hypoimmunogenic cell expresses or has increased expression and/or activity of a chimeric CD24/CD200 gene and/or gene product or a chimeric CD47/CD200 gene and/or gene product. In embodiments, the hypoimmunogenic cell expresses or has increased expression and/or activity of an MFG-E8 gene and/or gene product. In embodiments, the hypoimmunogenic cell expresses or has increased expression and/or activity of a NCAM gene and/or gene product. In embodiments, the hypoimmunogenic cell expresses or has increased expression and/or activity of an α-phagocytic integrin gene and/or gene product. In embodiments, the hypoimmunogenic cell expresses or has increased expression and/or activity of an antibody or antibody format molecule which targets an IL-6 surface receptor (anti-IL-6R).

In embodiments, the hypoimmunogenic cell expresses a FasL gene and/or gene product. In embodiments, the hypoimmunogenic cell does not overexpress a FasL gene and/or gene product.

In embodiments, the hypoimmunogenic cell substantially lacks expression and/or activity of one or more immunogenic proteins and expresses or has increased expression of one or more immunoprotective proteins.

In embodiments, the hypoimmunogenic cell has reduced or ablated expression and/or activity of 3 or more immunogenic proteins, 4 or more immunogenic proteins, 5 or more immunogenic proteins, 6 or more immunogenic proteins, 7 or more immunogenic proteins, 8 or more immunogenic proteins, 9 or more immunogenic proteins, 10 or more immunogenic proteins, 11 or more immunogenic proteins, or 12 or more immunogenic proteins. In embodiments, the hypoimmunogenic cell expresses or has increased expression of 3 or more immunoprotective proteins, 4 or more immunoprotective proteins, 5 or more immunoprotective proteins, 6 or more immunoprotective proteins, 7 or more immunoprotective proteins, 8 or more immunoprotective proteins, 9 or more immunoprotective proteins, or 10 or more immunoprotective proteins.

In embodiments, the hypoimmunogenic cell has reduced or ablated expression and/or activity of a gene and/or gene product of HLA-A, HLA-B, HLA-C, HLA-F, CIITA, IL-6, TRAC, TRBC, and one of either HLA-E or HLA-G.

In embodiments, the hypoimmunogenic cell has reduced or ablated expression and/or activity of a gene and/or gene product of HLA-A, HLA-B, HLA-C, HLA-F, CIITA, IL-6, TRAC, TRBC, SerpinB9, and one of either HLA-E or HLA-G.

In embodiments, the hypoimmunogenic cell has reduced or ablated expression and/or activity of a gene and/or gene product of HLA-A, HLA-B, HLA-C, HLA-F, CIITA, IL-6, TRAC, TRBC, SerpinB9, and one of either HLA-E or HLA-G.

In embodiments, the hypoimmunogenic cell comprises a hypoimmunogenic cell that has reduced or ablated expression and/or activity of a gene and/or gene product of HLA-A, HLA-B, HLA-C, HLA-F, CIITA, IL-6, TRAC, TRBC, SerpinB9, one of either HLA-E or HLA-G, and one or more of IL-4, IL-10, and IL-16.

In embodiments, the hypoimmunogenic cell expresses or has increased expression and/or activity of α-phagocytic integrin, CCL2, H2-M3, FasL, MFEG8, and PD-L1 and/or CTLA-4, wherein the hypoimmunogenic cell does not overexpress FasL, and wherein the hypoimmunogenic cell is not activated with the expression of PD-L1, and expresses or has increased expression and/or activity of one of either CD24, CD47, CD200, chimeric CD24/CD47, chimeric CD24/CD200, and chimeric CD47/CD200, or two of either CD24, CD47, and CD200.

In embodiments, the hypoimmunogenic cell expresses or has increased expression and/or activity of α-phagocytic integrin, CCL2, H2-M3, FasL, MFEG8, SerpinB9, and PD-L1 and/or CTLA-4, wherein the hypoimmunogenic cell does not overexpress FasL, and wherein the hypoimmunogenic cell is not activated with the expression of PD-L1, and expresses or has increased expression and/or activity of one of either CD24, CD47, CD200, chimeric CD24/CD47, chimeric CD24/CD200, and chimeric CD47/CD200, or two of either CD24, CD47, and CD200.

In embodiments, the hypoimmunogenic cell expresses or has increased expression and/or activity of a CD200 gene and/or gene product and does not express and/or substantially lacks either a CD24 or a CD47 gene and/or gene product. In embodiments, the hypoimmunogenic cell has no expression and/or activity of a SerpinB9 gene and/or gene product and a CD200 gene and/or gene product.

In embodiments, the hypoimmunogenic cell is allogeneic. In embodiments, the hypoimmunogenic cell does not cause an immune reaction in patients to which it or a BioNV derived therefrom is administered.

In embodiments, the hypoimmunogenic cell comprises one or more targeting agents. In embodiments, the one or more targeting agents comprises a chimeric antigen receptor (CAR). In embodiments, the CAR is bispecific. In embodiments, the CAR lacks an intracellular portion. In embodiments, the CAR comprises a targeting agent, a transmembrane domain, and an intracellular domain, which comprises a costimulatory domain and/or a signaling domain. In embodiments, the transmembrane domain is derived from CD28, CD3ζ, CD4, CD8a, or ICOS, or a fragment thereof. In embodiments, the intracellular domain comprises an intracellular signaling domain of a CD3ζ-chain and/or one or more co-stimulatory molecules, optionally selected from CD28, 4-1BB, ICOS, CD27, and OX40. In embodiments, the one or more targeting agents comprises an antibody or antibody format. In embodiments, the antibody or antibody format is selected from one or more of a monoclonal antibody, polyclonal antibody, antibody fragment, Fab, Fab′, Fab′-SH, F(ab′)2, Fv, single chain Fv (scFv), V_{NAR}, V_{H}H, afflilin, diabody, nanobody, linear antibody, bispecific antibody, multi-specific antibody, chimeric antibody, humanized antibody, human antibody, and fusion protein comprising the antigen-binding portion of an antibody. In embodiments, the antibody format is a scFv. In embodiments, the one or more targeting agents comprises a viral epitope recognition receptor (VERR) or viral ligand. In embodiments, the one or more targeting agents comprises a ligand for a receptor. In embodiments, the one or more targeting agents comprises a receptor for a ligand.

In embodiments, the hypoimmunogenic cell is a T cell; however, a person of skill in the art, with the benefit of this disclosure in its entirety, will appreciate that the hypoimmunogenic cell can be any cell type, and that activation can be via any cell receptor present on the cell. In embodiments, activating the hypoimmunogenic cell comprises activation of a TCR/CD3 receptor complex by a protein antigen. In embodiments, activating the hypoimmunogenic cell comprises activation of a TCR/CD3 receptor complex by a small molecule. In embodiments, the hypoimmunogenic cell comprises activation of a TCR/CD3 receptor complex by a viral antigen.

In embodiments, activating the hypoimmunogenic cell comprises activation of a calcium dependent channel. In embodiments, activating the hypoimmunogenic cell comprises activation of an LFA-1 integrin receptor. In embodiments, activating the hypoimmunogenic cell comprises activation of a CD28 receptor. In embodiments, activating the hypoimmunogenic cell comprises activation of an IL-2 receptor.

In embodiments, activating the hypoimmunogenic cell comprises activation of a SLAMF1(CD150) receptor. In embodiments, the activation of the SLAMF1(CD150) receptor is by a measles virus. In embodiments, the activation of the SLAMF1(CD150) receptor is by a Gram-negative bacteria.

In embodiments, activating the hypoimmunogenic cell comprises activation of an IFNγ receptor. In embodiments, activating the hypoimmunogenic cell comprises activation of a CD4 receptor by one or more viruses.

In embodiments, activating the hypoimmunogenic cell, such as a Natural Killer cell (or T-cell if the receptor(s) listed below is relevant to T-cell activation), comprises activation of a receptor (either individually or in combination to allow synergistic activation responses) such as CD2, CD3, CD16, CD28, CD314 (NKG2D), CD335, B7-H6, CD158d, IL-2R, IL-12R, DNAM-1, CD2, CD44, CD137, CX3CR1, CD27, CD160, 2B4 but not limited to these sensory receptors. CD16 can be activated via binding to the Fc region of antibodies (anti-S2 IgG). CD134, CD335, B7-H6 can be activated in combination with ICAM-1 combined with the Fc region of antibodies. CD314 can be activated by ULBP1, MICA, MICB, or H60. CD158d can be activated to trigger proinflammatory molecules via HLA-G. IL-2R can be activated by molecular IL-2. IL-12R can be activated by molecular IL-12. DNAM-1 can be activated by CD155 or CD112 or NKp30. CD2 can be activated by LFA3. CD44 can be activated by hyaluronic acid, hyaluronan, osteopontin, collagens, or matrix metalloproteinases. CD28 homolog can be activated by B7H7 protein ligand. CD137 can be activated by itself and members of the Tumor Necrosis Factor Receptor family of proteins. CX3CR1 can be activated by CX3CL1. CD27 can be activated by CD70. CD160 can be activated by HLA-C. 2B4 can be activated by CD48. Each type of activation can lead to differing levels of cytotoxic biomolecules, each of which may have therapeutic value when packages into a BioNV. The activation of each (or any) combination above can occur by the addition of the each of the following ligands (or any other relevant ligand) respectively, either in soluble form (partial peptide, domain or whole protein), anchored in a membrane (partial peptide, domain or whole protein), a target cell membrane constituent (partial peptide, domain or whole protein), or cross-linked to a solid phase such as, but not limited to, sephadex or magnetic beads (as a partial peptide, domain or whole protein).

In embodiments, the activation of some Natural Killer receptors (or T-cell if the receptor(s) listed below is relevant to T-cell activation), does not lead to degranulation or granule polarization to the IS. These receptors include, but are not limited to, i) 2B4 individually, ii) CD134 individually, iii) CD134 in combination with 2B4 and one of either inhibitory receptor KIR or CD94, iv) LFA-1 in combination with one of either inhibitory receptor KIR or CD94, v) CD16 in combination with one of either inhibitory receptor KIR or CD94. The activation of each (or any) combination above can occur by the addition of the each of the following ligands (or any other relevant ligand) respectively, either in soluble form (partial peptide, domain or whole protein), anchored in a membrane (partial peptide, domain or whole protein), a target cell membrane constituent (partial peptide, domain or whole protein), or cross-linked to a solid phase such as, but not limited to, sephadex or magnetic beads (as a partial peptide, domain or whole protein), i) CD48, ii) ULBP1, iii) HLA-E, iv) HLA-C in combination with HLA-E, or v) HLA-E.

In embodiments, the activation of some Natural Killer receptors (or T-cell if the receptor(s) listed below is relevant to T-cell activation), leads to degranulation. Immediately prior to degranulation, perforins and granzymes may (or may not) undergo post-translational modifications that enhance their activities, such as the binding of calcium to perforin, placing the protein in active state. It may be favorable to activate cells along the degranulation pathway to achieve maximum effectiveness of cytotoxic proteins. These receptors include but are not limited to, i) CD16 in combination with LFA-1 and one of either inhibitory receptor KIR or CD94, ii) CD134 in combination with 2B4, or iii) CD16 individually. The activation of each (or any) combination above can occur by the addition of the each of the following ligands (or any other relevant ligand) respectively, either in soluble form (partial peptide, domain or whole protein), anchored in a membrane (partial peptide, domain or whole protein), a target cell membrane constituent (partial peptide, domain or whole protein), or cross-linked to a solid phase such as, but not limited to, sephadex or magnetic beads (as a partial peptide, domain or whole protein), i) HLA-C in combination with HLA-E, ii) ULBP1 in combination with CD48, or iii) anti-S2 IgG.

In embodiments, receptors that when activated, lead to the polarization of lytic granules may not (or may be) be favorable, as their localization to the cell membrane may minimize their packaging in to BioNVs, but may maximize their packaging into secreted EVs (exosomes and microsomes), due to differences in extrusion manufacturing (BioNVs) vs. cellular exocytosis processing (EVs). Some receptors, that induce the polarization of lytic granules to the IS include but are not limited to, i) CD16 in combination with LFA-1 (also leads to degranulation), ii) LFA-1 individually, or iii) CD134 in combination with LFA-1 and 2B4 (also leads to degranulation). The activation of each (or any) combination above can occur by the addition of the each of the following ligands (or any other relevant ligand) respectively, either in soluble form (partial peptide, domain or whole protein), anchored in a membrane (partial peptide, domain or whole protein), a target cell membrane constituent (partial peptide, domain or whole protein), or cross-linked to a solid phase such as, but not limited to, sephadex or magnetic beads (as a partial peptide, domain or whole protein), i) anti-S2 IgG in combination with ICAM-1, ii) ICAM-1, or iii) ULBP1 in combination with CD48 and ICAM-1.

In embodiments, activating the hypoimmunogenic cell comprises activation by an engineered, non-native biomolecule selected from one or more of a soluble peptide, a chimeric antigen receptor, a small molecule decoy, a small molecule ligand, a designer nucleic acid ligand, a carbohydrate ligand, a viral ligand, a chimeric biomolecular ligand, a fusion protein, an antibody, and an antibody format molecule. In embodiments, activating the hypoimmunogenic cell comprises activation by an inorganic compound. In embodiments, activating the hypoimmunogenic cell comprises activation by expression, overexpression, or increased activity of a transcription factor.

In embodiments, activating the hypoimmunogenic cell comprises activation at the DNA level by one or more of a transposase-based method, Cre/Lox-based method, endonuclease-based method, homologous recombination (HR)-based method, non-homologous end joining (NEHJ)-based method, microhomology-mediated end-joining (MMEJ)-based method, homology-mediated end joining (HMEJ)-based method, small RNA, or a combination thereof. In embodiments, the small RNA comprises one or more of a guide RNA (gRNA), tracer RNA (tracrRNA), micro RNA (miRNA), RNA inference (RNAi), small interference RNA (siRNA), duplex RNA, Piwi-interacting RNA (piRNA), small nuclear RNA (snRNA), small nucleolar RNA (snoRNA), antisense oligonucleotide (ASO), locked nucleic acid (LNA), splice switching oligonucleotide (SSO), tRNA, complementary messenger RNA, repeat associated small interfering RNA (rasiRNA), endonuclease, and small non-coding RNA.

In embodiments, activating the hypoimmunogenic cell comprises activation at the RNA level by one or more guide RNA (gRNA), tracer RNA (tracrRNA), micro RNA (miRNA), RNA inference (RNAi), small interference RNA (siRNA), duplex RNA, Piwi-interacting RNA (piRNA), small nuclear RNA (snRNA), small nucleolar RNA (snoRNA), antisense oligonucleotide (ASO), locked nucleic acid (LNA), splice switching oligonucleotide (SSO), tRNA, complementary messenger RNA, repeat associated small interfering RNA (rasiRNA), endonuclease, small non-coding RNA, IRES element, or a combination thereof. In embodiments, activating comprises one or more RNA-guided endonucleases.

In embodiments, activating the hypoimmunogenic cell comprises activation by an endogenous promoter region and/or enhancer region. In embodiments, activating the hypoimmunogenic cell comprises activation by stably integrating a genetic element. In embodiments, activating the hypoimmunogenic cell comprises activation by transient expression of a genetic element.

In embodiments, activating the hypoimmunogenic cell results in a metabolically altered state of the hypoimmunogenic cell.

In embodiments, the one or more therapeutically relevant biomolecules is a chemokine, interferon, interleukin, alarmin, lymphokine, perforin, granzyme, granulysin, tumor necrosis factor (TNF), colony-stimulating factor, bone morphogenic protein (BMP), erythropoietin (EPO), granulocyte-stimulating factor (G-CSF), granulocyte macrophage colony-stimulating factor (GM-CSF), gene editing payload, fusion protein, antibody or antibody format, or a combination thereof. In embodiments, the cytokine is a pro-inflammatory cytokine. In embodiments, the cytokine is an anti-inflammatory cytokine. In embodiments, the granzyme is granzyme A, B, H, K, or M.

In embodiments, the gene editing payload comprises one or more gene editor nucleic acids and/or proteins, or one or more nucleic acids encoding one or more gene editors. In embodiments, the one or more gene editors is a site-directed endonuclease, TALEN, ZFN, RNase P RNA, CRISPR/Cas nuclease, C2c1, C2c2, C2c3, Cas9, Cpf1, TevCas9, Archaea Cas9, CasY.1, CasY.2, CasY.3, CasY.4, CasY.5, CasY.6, CasX, Cas omega, transposase, and/or any ortholog or homolog thereof. In embodiments, the gene editing payload comprises a transactivating response region (TAR) loop system.

In embodiments, the therapeutic BioNV comprises (i) one or more membrane-embedded targeting agents targeted against one or more cellular biomarkers, (ii) one or more membrane-embedded proteins of an α-phagocytic integrin, CCL2, H2-M3, FasL, MFEG8, PD-L1 and/or CTLA-4, SerpinB9, and anti-IL-6R antibody or antibody format, (iii) a membrane-embedded protein of one of either CD24, CD47, CD200, chimeric CD24/CD47, chimeric CD24/CD200, and chimeric CD47/CD200, or two of either CD24, CD47, and CD200, and (iv) a membrane substantially lacking proteins of one or more of HLA-A, HLA-B, HLA-C, HLA-F, CIITA, IL-6, TRAC, TRBC, one of either HLA-E or HLA-G, and one or more of IL-4, IL-10, and IL-16, wherein the one or more therapeutically relevant biomolecules is a chemokine, interferon, interleukin, alarmin, lymphokine, perforin, granzyme, granulysin, tumor necrosis factor (TNF), colony-stimulating factor, bone morphogenic protein (BMP), erythropoietin (EPO), granulocyte-stimulating factor (G-CSF), granulocyte macrophage colony-stimulating factor (GM-CSF), gene editing payload, fusion protein, antibody or antibody format, or a combination thereof.

In embodiments, the therapeutic BioNV comprises (i) one or more membrane-embedded targeting agents targeted against one or more cellular biomarkers, (ii) one or more membrane-embedded proteins of an α-phagocytic integrin, CCL2, H2-M3, FasL, MFEG8, PD-L1 and/or CTLA-4, SerpinB9, and anti-IL-6R antibody or antibody format, (iii) a membrane-embedded protein of either CD24 and CD47, or a chimeric CD24/CD47, and (iv) a membrane substantially lacking proteins of one or more of HLA-A, HLA-B, HLA-C, HLA-F, CIITA, IL-6, TRAC, TRBC, one of either HLA-E or HLA-G, SerpinB9 and CD200, and one or more of IL-4, IL-10, and IL-16, wherein the one or more therapeutically relevant biomolecules is a chemokine, interferon, interleukin, alarmin, lymphokine, perforin, granzyme, granulysin, tumor necrosis factor (TNF), colony-stimulating factor, bone morphogenic protein (BMP), erythropoietin (EPO), granulocyte-stimulating factor (G-CSF), granulocyte macrophage colony-stimulating factor (GM-CSF), gene editing payload, fusion protein, antibody or antibody format, or a combination thereof.

In embodiments, the one or more targeting agents is an antibody or antibody format. In embodiments, the one or more targeting agents is a CAR. In embodiments, processing the activated, hypoimmunogenic cell is by one or more of sonication, adaptive focused acoustics technology, French press, extrusion, serial extrusion, enzymatic rupture, cell lysis by detergent, and/or electroporation. In embodiments, processing the activated, hypoimmunogenic cell is by serial extrusion.

In embodiments, the therapeutic BioNV of about 10 nm to about 1200 nm in size. In embodiments, the therapeutic BioNV is about 10 nm to about 100 nm in size. In embodiments, the therapeutic BioNV is about 100 nm to about 200 nm in size. In embodiments, the therapeutic BioNV is about 200 nm to about 500 nm in size. In embodiments, the therapeutic BioNV is about 500 nm to about 1200 in size.

In embodiments, the therapeutic exosome comprises (i) one or more membrane-embedded targeting agents targeted against one or more cellular biomarkers, (ii) one or more membrane-embedded proteins of an α-phagocytic integrin, CCL2, H2-M3, FasL, MFEG8, PD-L1 and/or CTLA-4, SerpinB9, and anti-IL-6R antibody or antibody format, (iii) a membrane-embedded protein of one of either CD24, CD47, CD200, chimeric CD24/CD47, chimeric CD24/CD200, and chimeric CD47/CD200, or two of either CD24, CD47, and CD200, and (iv) a membrane substantially lacking proteins of one or more of HLA-A, HLA-B, HLA-C, HLA-F, CIITA, IL-6, TRAC, TRBC, one of either HLA-E or HLA-G, and one or more of IL-4, IL-10, and IL-16, wherein the one or more therapeutically relevant biomolecules is a chemokine, interferon, interleukin, alarmin, lymphokine, perforin, tumor necrosis factor (TNF), colony-stimulating factor, bone morphogenic protein (BMP), erythropoietin (EPO), granulocyte-stimulating factor (G-CSF), granulocyte macrophage colony-stimulating factor (GM-CSF), gene editing payload, fusion protein, antibody or antibody format, or a combination thereof.

In embodiments, the therapeutic exosome comprises (i) one or more membrane-embedded targeting agents targeted against one or more cellular biomarkers, (ii) one or more membrane-embedded proteins of an α-phagocytic integrin, CCL2, H2-M3, FasL, MFEG8, PD-L1 and/or CTLA-4, SerpinB9, and anti-IL-6R antibody or antibody format, (iii) a membrane-embedded protein of either CD24 and CD47, or a chimeric CD24/CD47, and (iv) a membrane substantially lacking proteins of one or more of HLA-A, HLA-B, HLA-C, HLA-F, CIITA, IL-6, TRAC, TRBC, one of either HLA-E or HLA-G, SerpinB9 and CD200, and one or more of IL-4, IL-10, and IL-16, wherein the one or more therapeutically relevant biomolecules is a chemokine, interferon, interleukin, alarmin, lymphokine, perforin, granzyme, granulysin, tumor necrosis factor (TNF), colony-stimulating factor, bone morphogenic protein (BMP), erythropoietin (EPO), granulocyte-stimulating factor (G-CSF), granulocyte macrophage colony-stimulating factor (GM-CSF), gene editing payload, fusion protein, antibody or antibody format, or a combination thereof.

In embodiments, the one or more targeting agents is an antibody or antibody format. In embodiments, the one or more targeting agents is a CAR. In embodiments, harvesting the therapeutic exosomes from the activated, hypoimmunogenic cell comprises inducing hypoxia. In embodiments, harvesting the therapeutic exosomes from the activated, hypoimmunogenic cell comprises expressing or increasing expression of one or more cellular factors. In embodiments, harvesting the therapeutic exosomes from the activated, hypoimmunogenic cell comprises supplying one or more small molecule exosome modulators. In embodiments, harvesting the therapeutic exosomes from the activated, hypoimmunogenic cell comprises supplementing the media with one or more exosome factors. In embodiments, the therapeutic exosome is about 10 nm to about 200 nm in size. In embodiments, the therapeutic exosome is about 10 nm to about 100 nm in size. In embodiments, the therapeutic exosome is about 100 nm to about 200 nm in size.

In embodiments, the mammalian disease is a cancer, infectious disease, hereditary disorder, or an orphan disease.

In aspects, described herein are methods of treating and/or preventing a disease or disorder using co-administration of a BioNV and whole cell. In embodiments, methods include administering a therapeutically effective amount of a biomimetic nanovesicle (BioNV) comprising one or more BioNV antigen-binding constructs, and administering a therapeutically effective amount of a whole cell comprising one or more whole cell antigen-binding constructs.

In embodiments, the disease or disorder is cancer. In embodiments, the cancer is one or more of a carcinoma, a sarcoma, a myeloma, a leukemia, a lymphoma, a mixed-type cancer, and/or a metastatic cancer.

In embodiments, the BioNV is administered first and the whole cell is administered second, the whole cell is administered first and the BioNV is administered second, or the BioNV and the whole cell are administered contemporaneously.

In embodiments, the one or more BioNV antigen-binding constructs comprises a CAR. In embodiments, the one or more whole cell antigen-binding comprises a CAR. In embodiments, the one or more whole cell antigen-binding constructs binds a cancer cell. In embodiments, the one or more BioNV antigen-binding constructs binds a cancer cell. In embodiments, the one or more whole cell antigen-binding constructs binds the BioNV. In embodiments, the one or more BioNV antigen-binding constructs binds the whole cell. In embodiments, the whole cell comprises a CAR that binds an antigen, ligand, or receptor present on both a cancer cell and the BioNV.

In embodiments, the BioNV comprises a CAR that binds an antigen, ligand, or receptor present on a cancer cell, and an antigen, ligand, and/or receptor that binds a CAR on the whole cell. In embodiments, the whole cell comprises a first CAR that binds an antigen, ligand, or receptor present on a cancer cell, and a second CAR that binds an antigen, ligand, and/or receptor present on the BioNV.

In embodiments, the antigen, ligand, and/or receptor on the BioNV is, comprises, or resembles, the antigen, ligand, or receptor present on the cancer cell; or the antigen, ligand, and/or receptor on the BioNV is different from the antigen, ligand, or receptor present on the cancer cell. In embodiments, the first CAR is capable of signaling via a pathway that results in cell-mediated cytotoxicity, or comprises one or more intracellular signaling domains capable of signaling via a pathway that results in cell-mediated cytotoxicity. In embodiments, second CAR is capable of signaling via a pathway that results in cell-mediated cytotoxicity by the whole cell, or comprises one or more intracellular signaling domains capable of signaling via a pathway that results in cell-mediated cytotoxicity. In embodiments, the second CAR is not capable of signaling via a pathway that results in cell-mediated cytotoxicity by the whole cell, or lacks one or more intracellular signaling domains capable of signaling via a pathway that results in cell-mediated cytotoxicity. In embodiments, the second CAR is capable of signaling via a pathway that results in persistence, survival, and/or proliferation of the whole cell, or comprises one or more intracellular signaling domains capable of signaling via a pathway that results in persistence, survival, and/or proliferation of the whole cell.

In embodiments, administering the BioNV improves the functionality of the whole cell. In embodiments, the functionality of the whole cell comprises one or more of cell-mediated cytotoxicity, cytokine release, tumor cell or cancer cell killing, honing to a tumor cell or cancer tissue, tissue infiltration, proliferation, persistence, and/or survival. In embodiments, administering the BioNV reduces one or more toxicities of the whole cell.

### DESCRIPTION OF THE DRAWINGS

FIG. 1 depicts a non-limiting diagrammatic representation of T cell activation via the interaction between the MHC class I T cell receptor (TCR) synapse. Target peptide display via MHC class I complexes on virally infected cells, cancer cells, etc., can be recognized by hypervariable regions of TCRα/β on lymphocytes, resulting in conformational changes of the intracellular domains that trigger signaling to produce cytotoxic cytokines.

FIG. 2 depicts a non-limiting diagrammatic representation of an activated immune cell containing anti-cancer cytokines processed into a biomimetic nanovesicle (BioNV). Anti-cytokines (and various cytotoxic peptides) can be loaded into BioNVs which can be used for robust delivery.

FIG. 3 depicts a non-limiting diagrammatic representation of a process of activating cells with a biomarker antigen via its CAR/TCR receptor. The biomarker antigen can be conjugated to a magnetic or streptavidin bead (or others), then added to the cellular suspension to cause activation. After activation, the antigen can be separated from the cellular suspension, leaving the activated cell with an unbound CAR/TCR complex.

FIGS. 4A-4D depict non-limiting diagrammatic representations of BioNV whole cell therapy co-administration strategies for treating cancer. FIG. 4A shows a BioNV+whole cell cancer cell targeting strategy, where the BioNV and whole cell (e.g., CAR-T cell) target a tumor cell via different antigen recognition constructs (e.g., CAR 1-CAR 1R and CAR 2-CAR 2R), and the whole cell recognizes both the tumor cell and BioNV via the same construct to improve cancer cell targeting. FIG. 4B shows a BioNV+whole cell cancer cell targeting strategy, where the BioNV and whole cell (e.g., CAR-T cell) target a tumor cell via different antigen recognition constructs (e.g., CAR 1-CAR 1R and CAR 2-CAR 2R), and the whole cell recognizes both the tumor cell and BioNV via the same antigen-binding domain, but the intracellular signaling domains differs (e.g., CAR 2_{A}) to improve cancer cell targeting and whole cell activation/stimulation with pathways that can be redundant, overlap, or differ. FIG. 4C shows a BioNV+whole cell cancer cell targeting strategy, where the BioNV and whole cell (e.g., CAR-T cell) target a tumor cell via different antigen recognition constructs (e.g., CAR 1-CAR 1R and CAR 2-CAR 2R), and the whole cell recognizes both the tumor cell and BioNV via different antigen-binding domains and the intracellular signaling domains differs between CAR 2 and CAR 2_{A}) to improve cancer cell targeting, whole cell activation/stimulation with pathways that can be redundant, overlap, or differ, and reduce off-target binding. FIG. 4D shows a similar strategy to FIG. 4C where a T cell receptor (TCR) can be used in place of (or in addition to) the CAR to target the whole cell (e.g., TCR 2) to the tumor cell, to target the whole cell to the BioNV (e.g., via TCR 2_{A}), and to target the BioNV to the tumor cell (e.g., TCR 1) to a cognate TCR ligand (e.g., TCR 1L) on the tumor cell.

FIG. 5 depicts a non-limiting diagrammatic representation of a serial extrusion to produce BioNVs.

### DETAILED DESCRIPTION OF THE DISCLOSURE

The present disclosure relates to, in part, methods of generating therapeutic biomimetic nanovesicles (BioNVs), or extracellular vesicles (EVs) such as naturally-shed therapeutic exosomes or microsomes, that find use in treating diseases. It should be understood that while exosomes are referred to below, any other EV can be used. The therapeutic BioNVs and/or therapeutic exosomes originate from hypoimmunogenic cells which have been modified to lack expression and/or activity of one or more immunogenic molecules (e.g., MHC class I/II, HLA, T cell receptor (TCR), cytokine release syndrome (CRS) molecules, etc.) and express and/or have increased expression of one or more immunoprotective molecules (e.g., CD47, CD24, CD200, α-phagocytic integrins, etc.). The hypoimmunogenic cells are subject to genetic regulation and activation to control expression of therapeutically relevant biomolecules which can be lumen-loaded, or encapsulated, within the BioNVs and exosomes. The therapeutic BioNVs and therapeutic exosomes can originate from any hypoimmunogenic cell type, including stem cell subsets or cells differentiated therefrom.

The present disclosure relates to, in part, compositions of BioNVs and/or exosomes with surface-oriented targeting agents (e.g., CARs) which recognize one or more cellular biomarkers. This results in a cell-targeted (or disease-specific) therapeutic BioNV/exosome on the order of 20-1200 nm in size, far smaller in size than a traditional cell-based CAR-T/NK cell therapies. The BioNV/exosome can originate from a cell such as a stem cell, iPSC, reprogrammed pluripotent or multipotent cell, embryonic stem cell, mesenchymal stem cell, or a differentiated cell from any stem cell, among other cell types. The plasma membrane-derived BioNV, or naturally-shed exosome, retains the hypoimmunogenic properties from the hypoimmunogenic cell. These properties can be obtained from a hypoimmunogenic cell due to genetic engineering focused on knock-out of specific immunogenic cell surface markers or immunogenic molecules (e.g., MHC class I/II, T cell receptor (TCR), cytokine release syndrome (CRS), etc.) and/or expression or increased expression of immunoprotective cell surface markers (e.g., CD47, CD34, CD24, CD200, α-phagocytic, etc.). The targeting agent can comprise all variations of an antibody construct, including for example, Fab, Fab′, Fab′-SH, F(ab′)2, scFv, diabody, nanobody, linear antibody, bispecific antibody, multi-specific antibody, chimeric antibody, humanized antibody, human antibody, and fusion proteins comprising the antigen-binding portion of an antibody, V_{H}H nanobodies, V_{NARS}, among other antibody formats, allowing BioNVs/exosomes to target of any cell of interest (targeting any type of cell surface biomarker). The BioNV/exosome can comprise a targeting agent that is a viral epitope recognition receptor (VERR) or viral ligand, a ligand for a receptor, or a receptor for a ligand. The BioNV/exosome can also encapsulate and deliver any small molecule, biologic, nucleic acid, gene editing therapeutic payload, etc., of choice to the intended cellular targets to treat cancer.

Moreover, the present disclosure relates to, in part, methods of treating or preventing a disease or disorder by administering the therapeutically effective amount of a therapeutic BioNV or a therapeutic exosome to a subject in need thereof.

Methods of Generating Biomimetic Nanovesicles (BioNVs) and/or Exosomes

In aspects, the present disclosure includes methods or generating a therapeutic biomimetic nanovesicle (BioNV) comprising (a) obtaining a hypoimmunogenic cell, (b) activating the hypoimmunogenic cell to express one or more therapeutically relevant biomolecules, and (c) processing the activated, hypoimmunogenic cell to generate the therapeutic BioNV, wherein the therapeutic BioNV comprises the hypoimmunogenic cell plasma membrane and encapsulates the one or more therapeutically relevant biomolecules.

In aspects, the present disclosure includes methods of generating therapeutic exosomes comprising (a) obtaining a hypoimmunogenic cell, (b) activating the hypoimmunogenic cell to express one or more therapeutically relevant biomolecules, and (c) harvesting naturally shed therapeutic exosomes from said activated, hypoimmunogenic cell, wherein the therapeutic exosomes comprise the hypoimmunogenic cell plasma membrane and encapsulates the one or more therapeutically relevant biomolecules.

In embodiments, therapeutic BioNVs and/or exosomes are derived from hypoimmunogenic cells, wherein the hypoimmunogenic cell can be a stem cell (e.g., iPSC, mesenchymal, etc.), an induced pluripotent stem cell (iPSC), a reprogrammed pluripotent or multipotent cell, an embryonic stem cell, a mesenchymal stem cell, or a differentiated cell which originates from any modified cell thereof. In embodiments, the hypoimmunogenic cell from which BioNVs and/or exosomes are derived is a T cell, helper T cell, T-memory cell, or NK cell. In embodiments, the hypoimmunogenic cell is a macrophage. In embodiments, the cell is a monocyte. In embodiments, the hypoimmunogenic cell is a hepatocyte, a cardiomyocyte, a neuron, an endothelial cell, a pancreatic cell, or a retinal pigmented epithelium (RPE) cell. In embodiments, the hypoimmunogenic cell can be any terminally differentiated cell, for example and without limitation, a muscle cell (satellite cell), adipocyte, osteocyte, cardiomyocyte, hepatocyte, blood cell (including erythrocyte, thrombocyte, and all immune cell types), glial cell (among other neuronal cell types), epithelial cell, epidermal cell, interstitial cell (e.g., respiratory interstitial cell), fibroblast (e.g., dermal fibroblast), endothelial cell (e.g., bronchial endothelial cell), oral cell, stromal cell, or germ cell. In embodiments, the hypoimmunogenic cell can be any function-specific cell type, for example and without limitation, exocrine secretory epithelial cell, hormone-secreting cell (e.g. enteroendocrine cell, thyroid cell, pancreatic islet cell, etc.), sensory transducer cell, autonomic neuronal cell, sensory organ cell (e.g., pillar cell, olfactory cell, Schwann cell, satellite glial cell, etc.), barrier cell (e.g., pneumocyte, duct cell, kidney cell, podocyte, etc.), extracellular matrix cell (e.g., tendon fibroblast, osteoblast, connective tissue cell, etc.), or contractile cell (e.g., skeletal muscle cell, cardiac muscle cell, myoepithelial cell, etc.).

In embodiments, the modified cell is an iPSC. In embodiments, the iPSC is differentiated into a particular cell. In embodiments, the modified cell is differentiated to express or not express a variety of cell surface markers. In embodiments, the methods include BioNVs derived from a hypoimmunogenic iPSC.

In embodiments, therapeutic BioNVs/exosomes are derived from iPSCs that have been engineered to be hypoimmunogenic. In embodiments, iPSCs are reverted from a somatic state using microRNA technology in lieu of small molecule trans-activators. The use of microRNA provides a tighter differentiation system and that results in higher quality iPSCs. Without wishing to be bound by theory, these high quality iPSCs are less prone to expression dampening (of post-engineered proteins, such as CD47) and genetic drift, and possess higher culture splitting qualities/quantities (the cultures can be divided more times than other methods before cellular integrity issues occur).

In embodiments, the hypoimmunogenic cell from which the BioNVs and/or exosomes are derived retain functionality from the hypoimmunogenic cell, for example and without limitation, the ability to cross the blood-brain barrier, such as is the case of macrophages/monocytes, or tissue-specific factors such as is the case in cardiomyocytes, hepatocytes, etc.

In embodiments, the allogeneic and hypoimmunogenic properties of the activated cells (e.g., derived from iPSCs) are created by knocking-out, silencing, inactivating, blocking or otherwise negating the transcriptional efficiencies of immunogenic molecules. In embodiments, the hypoimmunogenic cell substantially lacks one or more MHC class I proteins, MHC class II proteins, T cell receptor (TCR) proteins, and/or cytokine release syndrome (CRS) proteins. Inactivation of immunogenic molecules can include, for example, disruption of MHC class I and beta-2-microglobulin (B2M) genes, such as in the case for CD8+ T cell lineages, and MHC class II and MHC II transactivator (CIITA) genes, such as in the case of CD4+ T cell lineages. Without wishing to be bound by theory, these proteins contribute to the human leukocyte antigen (HLA) immunogenicity that requires HLA allele matching in the donor-recipient for treatment by cell-based therapies. In embodiments, allogeneic and hypoimmunogenic properties are achieved by disruption of genes encoding the T cell receptor (TCR) proteins including, for example, the α and β chains (as in the case of αβ T cells) or the γ and δ chains (as in the case of γδ T cells) forming the ligand-binding site and the signaling modules CD3δ, CD3γ, CD3ε, and CD3ζ. This can be performed to reduce extraneous T cell receptor types other than those of the CAR cassette and further improve the homogeneity of the CAR of interest and reduce off-target effects in BioNV/exosome formation.

In embodiments, the hypoimmunogenic cell includes a knock-out, or disruption, of expression of immunogenic cell surface proteins and/or intracellular or secreted proteins. In embodiments, the hypoimmunogenic cell has a β2-macroglobulin (B2M) gene disruption and/or a disruption that reduces or ablates MHC class I protein expression and/or activity. In embodiments, knocking-out the B2M genes reduces the number of potential doses to be administered due to the risk of preventing long term acceptance of the BioNVs by the recipient, such as what has been observed in the whole cell-based approaches described above. To overcome this issue, in embodiments, the HLA-E or HLA-G gene remains intact, allowing the immune system to adapt to the resulting BioNV. In embodiments, the HLA-A, HLA-B, HLA-C, HLA-F, and HLA-E or HLA-G (but not both of HLA-E and HLA-G) are knocked out sequentially.

In embodiments, the hypoimmunogenic cell has an HLA-A gene disruption and/or a disruption that reduces or ablates HLA-A protein expression and/or activity. In embodiments, the hypoimmunogenic cell has an HLA-B gene disruption and/or a disruption that reduces or ablates HLA-B protein expression and/or activity. In embodiments, the hypoimmunogenic cell has an HLA-C gene disruption and/or a disruption that reduces or ablates HLA-C protein expression and/or activity. In embodiments, the hypoimmunogenic cell has an HLA-E gene disruption or an HLA-G gene disruption and/or a disruption that reduces or ablates HLA-E or HLA-G protein expression and/or activity. In embodiments, the hypoimmunogenic cell has an HLA-F gene disruption and/or a disruption that reduces or ablates HLA-F protein expression and/or activity.

In embodiments, the hypoimmunogenic cell has a CIITA gene disruption and/or a disruption that reduces or ablates MHC class II protein expression and/or activity. In embodiments, allogeneic iPSCs have their MHC Class I and MHC Class II complexes disrupted by knocking out critical proteins involved in their expression, for example, B2M which is a serum protein found in association with the MHC class I heavy chain on the surface of nearly all nucleated cells that is involved in the presentation of peptide antigens to the immune system. In embodiments, allogeneic iPSCs have their CIITA gene disrupted, so that a resulting differentiated cell line (e.g., DCs, mononuclear phagocytes, endothelial cells, thymic epithelial cells, B cells, etc.) does not express MHC class II proteins.

In embodiments, the hypoimmunogenic cell has a T cell alpha constant (TRAC) gene disruption and/or a disruption that reduces or ablates TRAC protein expression and/or activity. In embodiments, the hypoimmunogenic cell has a T cell beta constant (TRBC) gene disruption and/or a disruption that reduces or ablates TRBC protein expression and/or activity. In embodiments, the hypoimmunogenic cell has reduced or ablated expression of a PD-1 gene and/or reduced or ablated PD-1 protein expression and/or activity, wherein the hypoimmunogenic cell is activated; or the hypoimmunogenic cell has expression or increased expression of a PD-1 gene and/or gene product, wherein the hypoimmunogenic cell is not activated.

In embodiments, “increased expression and/or activity,” as used herein refers to an increase in expression and/or activity in the hypoimmunogenic cell in comparison to its native, or wild-type cognate cell. For example, in embodiments, the increased expression and/or activity of one or more biomolecules described herein can confer the hypoimmunogenic properties of an iPSC relative to an iPSC which does not have the same expression pattern or expression level of the protein. In embodiments, the “increased expression and/or activity,” is due to a genetic amendment, such as a knock-in.

CRS is a major concern with whole cell therapies, where despite engineered hypoimmunogenicity, effector functions and other consequences of interaction with cells post-infusion can result in the release of biomolecules that result in a systemic inflammatory syndrome characterized by fever, multiple organ dysfunction, etc. In embodiments, the hypoimmunogenic cell is engineered to disrupt expression and/or activity one or more proteins that contribute to CRS. In embodiments, the hypoimmunogenic cell has reduced or ablated expression and/or activity (e.g., knock-out or silencing) of CRS-related cytokines.

In embodiments, the hypoimmunogenic cell has an IL-4 gene disruption and/or a disruption that reduces or ablates IL-4 protein expression and/or activity. In embodiments, the hypoimmunogenic cell has an IL-6 gene disruption and/or a disruption that reduces or ablates IL-6 protein expression and/or activity. In embodiments, the hypoimmunogenic cell has an IL-10 gene disruption and/or a disruption that reduces or ablates IL-10 protein expression and/or activity. In embodiments, the hypoimmunogenic cell has an IL-16 gene disruption and/or a disruption that reduces or ablates IL-16 protein expression and/or activity. In embodiments, the modified cell contains gene disruptions (e.g., knock-outs) in other cytokine release syndrome (CRS)-related interleukins. In embodiments, the reduction or ablation of interleukins causing CRS decreases the likelihood of CRS.

Serine proteinase inhibitor B9 (SerpinB9) is a member of the serine protease inhibitor superfamily. SerpinB9 has been reported to protect cells from the immune killing effects of granzyme B. In embodiments, the hypoimmunogenic cell has a SerpinB9 gene disruption and/or a disruption that reduces or ablates SerpinB9 protein expression and/or activity. In embodiments, the hypoimmunogenic cell has SerpinB9 knocked-out and/or silenced. Alternatively, in embodiments, the hypoimmunogenic cell expresses or has increased expression and/or activity of a SerpinB9 gene and/or gene product.

In embodiments, the overexpression of SerpinB9 sequesters the function of granzyme B which is related to immunostimulatory responses, such as apoptosis of a targeted and/or diseased cell. In embodiments, granzyme B is inhibited in activated lymphocytes, NK cells, macrophages, and follicular DCs, among other cell types. In embodiments, e.g., where a BioNV that is intended to deliver a non-granzyme payload, for example a gene editing payload, the hypoimmunogenic cell can express and/or overexpress SerpinB9.

In embodiments, the hypoimmunogenic cell expresses or has increased expression and/or activity of a CCL2 gene and/or gene product. In embodiments, the hypoimmunogenic cell expresses or has increased expression of a PD-L1 gene and/or gene product, and wherein the hypoimmunogenic cell is not activated; or wherein the modified cell has reduced or ablated expression of a PD-L1 gene and/or gene product, and wherein the hypoimmunogenic cell is activated. In embodiments, the hypoimmunogenic cell expresses or has increased expression and/or activity of a H2-M3 gene and/or gene product. In embodiments, the hypoimmunogenic cell expresses or has increased expression and/or activity of a CTLA-4 gene and/or gene product.

In embodiments, the hypoimmunogenic cell expresses or has increased expression and/or activity of a CD47 gene and/or gene product. In embodiments, prevention of the potential inhibitory phenotypes of CD47 expression across cells is done via interference with the inhibitory mechanism of action of the series of microRNAs on the 3′UTR of the CD47 gene by deleting this region in stable constructs or by eliminating/inhibiting the expression of the microRNAs. In embodiments, this can resolve inhibitory issues caused by the microRNAs.

In embodiments, the hypoimmunogenic cell expresses or has increased expression and/or activity of a CD24 gene and/or gene product. CD24 is a sialoglycoprotein expressed on mature granulocytes and B-cells and is also an anti-phagocytic protein. CD24 prevents phagocytosis through interactions with Siglec-G/10 on macrophages. In embodiments, the hypoimmunogenic cell expresses or overexpresses a CD24 protein and/or gene product. In embodiments, the hypoimmunogenic cell expresses or has increased expression and/or activity of a chimeric CD24/CD47 gene and/or gene product. In embodiments, the hypoimmunogenic cells include CD24/CD47, with a tethered transmembrane domain. In embodiments, the domains of CD47 isoform 2 and CD24 can be either separately expressed or tethered to form a bilobed, chimeric protein. In embodiments, the hypoimmunogenic cells are iPSCs are from fibroblasts, not from ABO cells.

In embodiments, the hypoimmunogenic cell expresses or has increased expression and/or activity of a CD200 gene and/or gene product. In embodiments, CD200 tags minimize phagocytosis by macrophages and also prevent the activation of granulocytes. In embodiments, the hypoimmunogenic cell does not express CD200 when it is not desirable to prevent granulocytes, for example in the solid tumor microenvironment (TME), as activation of granulocytes would complement the mechanism of action of a BioNV designed to release granzymes and perforins. However, in embodiments, if a CD47 or CD24 tag is used, or a CD24/CD47 chimeric, bilobed protein tag (each prevents phagocytosis) in combination with overexpressed H2-M3 (which dampens the NK response) is used, stability can be achieved without CD200, while allowing BioNV clearance. In embodiments, where granzymes and perforins are not a therapeutic biomolecule of choice, CD200 can be expressed to prevent the activation of granulocytes, while eliminating a CD47 tag or a CD24 tag, but not both tags. In embodiments, the hypoimmunogenic cell expresses or has increased expression and/or activity of a chimeric CD24/CD200 gene and/or gene product or a chimeric CD47/CD200 gene and/or gene product. In embodiments, these CD200 strategies represent a hypoimmunogenic cell line for generating BioNVs for targeting non-cancer cells, such as targeting the liver, kidney, cardiac cells, and/or tissue regeneration pathways.

In embodiments, the obtained hypoimmunogenic cell (or differentiated cell therefrom) does not express and/or overexpress all three of CD47, CD24, and CD200. In embodiments, the hypoimmunogenic cell is engineered such that BioNVs or exosomes that result from the hypoimmunogenic cell line are stabilized, but not to a degree where the BioNVs or exosomes are resistant to being cleared from the body. A BioNV/exosome that is too stable could eventually trigger a humoral response, resulting in limiting the number of doses or treatments that can be administered.

In embodiments, the hypoimmunogenic cell expresses or has increased expression and/or activity of an MFG-E8 gene and/or gene product. In embodiments, the hypoimmunogenic cell expresses or has increased expression and/or activity of a NCAM gene and/or gene product. In embodiments, the hypoimmunogenic cell expresses or has increased expression and/or activity of an α-phagocytic integrin gene and/or gene product.

In embodiments, the hypoimmunogenic cell expresses or has increased expression and/or activity of an antibody or antibody format molecule which targets an IL-6 surface receptor (anti-IL-6R). In embodiments, the hypoimmunogenic cell is an iPSC cell line that has an anti-IL-6R antibody engineered into the cell line. In embodiments, BioNVs/exosomes carry α-IL-6R, thereby blocking the signaling pathway on localized immune cells in the tumor environment from becoming activated.

In embodiments, the hypoimmunogenic cell expresses a FasL gene and/or gene product. In embodiments, the hypoimmunogenic cell does not overexpress a FasL gene and/or gene product. In embodiments, the hypoimmunogenic cell does not overexpress FasL because an enrichment of naturally expressed levels of FasL is observed in the membranes of BioNVs after processing, e.g., via serial extrusion. Too high of concentrations of FasL can be counter-productive and prevent the recruitment of T-cells to the solid tumor and/or cause premature T-cell death.

In embodiments, the hypoimmunogenic cells can express one or more fusion proteins of one or more portions of any immunoprotective protein herein. For example, in embodiments, constructs can be made where the appropriate portion of a ligand of choice is tethered to a transmembrane domain. In embodiments, constructs can be made where the biologically relevant portion of two or more proteins are tethered together and/or to a transmembrane domain.

In embodiments, the hypoimmunogenic cell substantially lacks expression and/or activity of one or more immunogenic proteins and expresses or has increased expression of one or more immunoprotective proteins.

In embodiments, the hypoimmunogenic cell has reduced or ablated expression and/or activity of one or more immunogenic proteins, such as proteins that result in an immune response in the subject, donor-recipient mismatch, HLA alloimmunity, inflammation, CRS, and the like, such as MHC class I proteins, MHC class II proteins, HLA proteins, TCR proteins, CRS proteins, etc. In embodiments, the hypoimmunogenic cell has reduced or ablated expression and/or activity of 3 or more immunogenic proteins, 4 or more immunogenic proteins, 5 or more immunogenic proteins, 6 or more immunogenic proteins, 7 or more immunogenic proteins, 8 or more immunogenic proteins, 9 or more immunogenic proteins, 10 or more immunogenic proteins, 11 or more immunogenic proteins, or 12 or more immunogenic proteins.

In embodiments, the modified cell has expression or increased expression and/or activity of one or more immunoprotective proteins, such as proteins that result prevent or reduce an immune response in the subject, prevent or reduce premature clearance of the BioNV in the subject, prevent or reduce phagocytosis, confer barrier-crossing functionality, and the like, such as CD47, CD24, CD200, CD34, CCL2, H2-M3, MFEG8, PD-L1 (non-activated cell source), CTLA-4, etc. In embodiments, the hypoimmunogenic cell expresses or has increased expression of 3 or more immunoprotective proteins, 4 or more immunoprotective proteins, 5 or more immunoprotective proteins, 6 or more immunoprotective proteins, 7 or more immunoprotective proteins, 8 or more immunoprotective proteins, 9 or more immunoprotective proteins, or 10 or more immunoprotective proteins.

In embodiments, the hypoimmunogenic cell has reduced or ablated expression and/or activity of a gene and/or gene product of HLA-A, HLA-B, HLA-C, HLA-F, CIITA, IL-6, TRAC, TRBC, and one of either HLA-E or HLA-G.

In embodiments, the hypoimmunogenic cell has reduced or ablated expression and/or activity of a gene and/or gene product of HLA-A, HLA-B, HLA-C, HLA-F, CIITA, IL-6, TRAC, TRBC, SerpinB9, and one of either HLA-E or HLA-G.

In embodiments, the hypoimmunogenic cell comprises a hypoimmunogenic cell that has reduced or ablated expression and/or activity of a gene and/or gene product of HLA-A, HLA-B, HLA-C, HLA-F, CIITA, IL-6, TRAC, TRBC, SerpinB9, one of either HLA-E or HLA-G, and one or more of IL-4, IL-10, and IL-16.

In embodiments, the hypoimmunogenic cell expresses or has increased expression and/or activity of α-phagocytic integrin, CCL2, H2-M3, FasL, MFEG8, and PD-L1 and/or CTLA-4, wherein the hypoimmunogenic cell does not overexpress FasL, and wherein the hypoimmunogenic cell is not activated with the expression of PD-L1, and expresses or has increased expression and/or activity of one of either CD24, CD47, CD200, chimeric CD24/CD47, chimeric CD24/CD200, and chimeric CD47/CD200, or two of either CD24, CD47, and CD200.

In embodiments, the hypoimmunogenic cell expresses or has increased expression and/or activity of α-phagocytic integrin, CCL2, H2-M3, FasL, MFEG8, SerpinB9, and PD-L1 and/or CTLA-4, wherein the hypoimmunogenic cell does not overexpress FasL, and wherein the hypoimmunogenic cell is not activated with the expression of PD-L1, and expresses or has increased expression and/or activity of one of either CD24, CD47, CD200, chimeric CD24/CD47, chimeric CD24/CD200, and chimeric CD47/CD200, or two of either CD24, CD47, and CD200.

In embodiments, the hypoimmunogenic cell expresses or has increased expression and/or activity of a CD200 gene and/or gene product and does not express and/or substantially lacks either a CD24 or a CD47 gene and/or gene product. In embodiments, the hypoimmunogenic cell has no expression and/or activity of a SerpinB9 gene and/or gene product and a CD200 gene and/or gene product.

In embodiments, the hypoimmunogenic cell expresses or has increased expression and/or activity of one or more antigen, ligand, and/or receptor of a tumor cell or cancer tissue. In embodiments, the one or more antigen, ligand, and/or receptor of a tumor cell or cancer tissue is listed in Table 4, Table 5, and/or Table 6 below. In embodiments, the one or more antigen, ligand, and/or receptor of a tumor cell or cancer tissue is a neoantigen, such as a mutant variant or peptide specific to a cancer or disease state that is otherwise not present in a subject to be treated. In embodiments, the hypoimmunogenic cell expresses or has increased expression and/or activity of a co-stimulatory molecule for immune cells. In embodiments, the co-stimulatory molecule for immune cells is IL-15.

In embodiments, the hypoimmunogenic cell is allogeneic. In embodiments, the hypoimmunogenic cell does not cause an immune reaction in patients to which it or a BioNV derived therefrom is administered.

In embodiments, the hypoimmunogenic cell comprises one or more targeting agents. In embodiments, the one or more targeting agents comprises a chimeric antigen receptor (CAR). In embodiments, the CAR is bispecific. In embodiments, the CAR lacks an intracellular portion. In embodiments, the CAR comprises a targeting agent, a transmembrane domain, and an intracellular domain, which comprises a costimulatory domain and/or a signaling domain. In embodiments, the transmembrane domain is derived from CD28, CD3ζ, CD4, CD8a, or ICOS, or a fragment thereof. In embodiments, the intracellular domain comprises an intracellular signaling domain of a CD3ζ-chain and/or one or more co-stimulatory molecules, optionally selected from CD28, 4-1BB, ICOS, CD27, and OX40.

In embodiments, the one or more targeting agents comprises an antibody or antibody format. In embodiments, In embodiments, the antibody or antibody format is selected from one or more of a monoclonal antibody, polyclonal antibody, antibody fragment, Fab, Fab′, Fab′-SH, F(ab′)2, Fv, single chain Fv (scFv), V_{NAR}, V_{H}H, afflilin, diabody, nanobody, linear antibody, bispecific antibody, multi-specific antibody, chimeric antibody, humanized antibody, human antibody, and fusion protein comprising the antigen-binding portion of an antibody. In embodiments, the antibody format is a scFv. In embodiments, the one or more targeting agents comprises a viral epitope recognition receptor (VERR) or viral ligand. In embodiments, the one or more targeting agents comprises a ligand for a receptor. In embodiments, the one or more targeting agents comprises a receptor for a ligand.

In embodiments, the obtained hypoimmunogenic cell has a B2M knock-out (KO), CIITA KO, CD47tg knock-in (KI), IL-6 KO are engineered into the iPSC, the TRAC and TRBC genes can be knocked out. In embodiments, only one gene for each is knocked out rather than both of on the separate alleles. TRAC and TRBC genes can be knocked out as described herein. The purpose of knocking out the TRAC and TRBC genes is to eliminate the T-cell receptors. In embodiments, the modified cell is differentiated to a T-cell subset which lacks T-cell receptors to derive the BioNVs/exosomes. Genetically modified the cells to substantially lack TCRs reduces the chances for a competing ligand to the CAR construct that can target non-specifically to alternate tissues. Therefore, in embodiments, the TCR genes are knocked-out as a strategy to reduce off-target effects of the BioNVs/exosomes. In embodiments, TRAC/TRBC knock-outs decrease the likelihood of CRS, as well as BioNV/exosome toxicity, generally.

In embodiments, the generated BioNVs/exosomes expresses CD47. Exosomes and vesicles are readily cleared from the body by macrophages through phagocytosis. Phagocytosis greatly impacts the therapeutic value and efficacy of exosomes. To prevent macrophage depletion of BioNVs/exosomes but without wishing to be bound by theory, in embodiments, the BioNV/exosome is CD47 tagged on the surface. A CD47tg (tag) provides a “do not eat me” signal which, in embodiments, increases the half-life and serum stability of the BioNV/exosome in the subject. In embodiments, the molecular CD47 isoform 2 (isoform that interacts with the SIRPα receptor on a macrophage) is engineered into the modified cell (e.g., iPSC cell line). Without the CD47tg, the BioNV/exosome half-life would be diminished due to phagocytosis inhibition, resulting in the need for higher and/or more frequent doses.

In embodiments, the obtained hypoimmunogenic cell has a B2M gene disruption (or other genes that can achieve the MHC I suppression), CIITA gene disruption (or other genes that achieve the MHC II suppression), CD47tg knock-in, and knock-outs of key Interleukins (ILs) that are related to the cause of cytokine release syndrome. In embodiments, the modified cell comprises an IL-6 gene disruption. Interleukin 6 (IL-6) is the main interleukin that is responsible or CRS. In embodiments, IL-6 knock-out prevents undesirable IL-6 packaging into the BioNV/exosome and reduces the BioNV's (or exosome's) contribution to a localized (and concentrated due to biomarker targeting) and/or potentially systemic CRS events.

In embodiments, the modified cell is expanded after engineering; any small scale expansion or large-scale feeder system expansion methods known in the art can be used.

In embodiments, the hypoimmunogenic cell for generating BioNVs/exosomes expresses one or more of the above proteins by a regulatable expression element, which can be modulated throughout the BioNV/exosome manufacturing process. In embodiments, regulatable expression element includes a regulatable promoter (such as Tet on/off promoter), or CRISPRa/i regulated systems, among other regulatable expression elements. In embodiments, CAR expression is controlled by a regulatable expression element. Simple over expression of the construct from a CMV (or other type) promoter can lead to surface densities of CAR that are too high and thus could cause a number of problems such as ‘hyper activation’ leading to in vitro exhaustion and cell death after activation. Cell death results in loss of the cell line for generating BioNVs/exosomes. In embodiments, the surface density of CAR protein constructs is regulated. For example, in embodiments, the typical concentration range of CAR protein per microgram of T cells is between 0.20 ng-0.70 ng. Too little expression of the CAR results in poor biomarker targeting. However, the density cannot exceed those of the cellular limit. Too high of a CAR density can result in parent cell exhaustion during the activation process in vitro prior to BioNV/exosome derivation and can also result in poor quality BioNV/exosomes due to high protein concentration in the plasma membrane. An upper limit allows for increasing the targeting efficiency for low biomarker expression on cancer cells. The density limit for cellular therapeutics is listed in the published U.S. non-provisional application, US 20220040106 A1, which is hereby incorporated by reference.

In embodiments, the hypoimmunogenic cell for generating BioNVs/exosomes can comprise stable cell integration (safe harbor genetic location) of any genetic element described herein in a cell (e.g., iPSCs) can be controlled by implementing a Tet-regulated CRISPRa+targeted 3× transcription factor targeted gRNA system. The CRISPR activation system for three upstream transcription factors can trigger a signal cascade event that enhances the production of CARs that have replaced endogenous antibody ORFs at designated locus (loci). This system can be ‘tunable’ by including a Tet-regulated promoter, allowing for the ability to vary the concentrations of CARs on the surface of the cell. Next, stable cell replacement of CDRs and heavy and light antibody regions with CAR cassettes can be achieved via Cpf-1 directed homology directed repair (HDR). Finally, the stably integrated CAR cassette can contain flanking gRNA binding sites which allow the scFV (among other antibody formats) or VERR/viral ligand to be repeatedly swapped or altered for rapid and consistent insertion of a desired sequence.

In embodiments, the concentration of the CAR on the surface of the iPSC base cell line, or any downstream differentiated cell (and the resulting BioNV/exosomes), can be regulated using a variety of transcription control elements, such as a tetracycline on/off promoter (or similar drug-regulated promoters) to drive the expression of a CRISPR activation/gRNA (CRISPRa) system. The CRISPRa system can then activate the antibody-regulating transcription factors, for example, Drm2, Fr5, and Bxp2, which regulate the expression of an engineered CAR cassette that has been integrated at the site of an antibody locus (where the antibody genes have been replaced). Additionally, a similar transcription control element can be provided to control overexpression of genes (e.g., CD47), drive genes controlling differentiation, etc., at defined manufacturing stages.

In embodiments, the hypoimmunogenic cell has the B2M KO, CIITA KO, CD47tg KI, IL-6 KO, where IL-2p GFP reporters are constructed, the CAR constructs can be integrated/engineered into the modified cell. In embodiments, the CAR constructs can be knocked-in to the TRAC/TRBC genes, simultaneously knocking-out the remaining TRAC/TRBC genes, resulting in a cell that is CAR+ and TRAC/TRBC−/−. In embodiments, the CAR construct can be knocked-in to the TRAC/TRBC gene location on both loci simultaneously, resulting in a cell that is CAR+/+ and TRAC/TRBC −/−.

In embodiments, the hypoimmunogenic cell has the B2M KO, CIITA KO, CD47tg KI, IL-6 KO, IL-2p GFP and CAR modified cells (e.g., iPSCs) engineered, and the Immunological Synapse (IS) quality is measured between the CAR recognition domains and the biomarker during the method of generating BioNVs/exosomes. In embodiments, the quality of the IS of BioNVs/exosomes can be directly related to efficacy in whole cell therapies.

In embodiments, the therapeutic BioNV/exosome, or the modified/activated cell derived therefrom, comprises a nucleic acid encoding green fluorescence protein (GFP). In embodiments, once the B2M KO, CIITA KO, CD47tg KI, IL-6 KO, TRAC/TRBC single KOs are engineered into the iPSC, a GFP molecule can be engineered into the modified cell line. In embodiments, this serves as the control cell line. In embodiments, the non-control cell line (the therapeutic cell line) does not have GFP. In embodiments, the nucleic acid encoding GFP is operably linked to a promoter from one or more of IL-2, perforin, granzyme, granulysin, alarmin, TNF, INF, a combination thereof, and/or any other cell-specific gene or reporter gene. The IL-2 promoter is constitutively activated when lymphocytes are broadly/globally activated from various stimuli. In embodiments, a more focused activation/repression (regulation) is used. In embodiments, the IL-2p GFP reporter gene serves as an indicator for the degree of broad/global activation of the cell (part of the BioNV/exosome derivation process). In embodiments, the GFP signal, coupled with immunoblot analysis of cytokine levels (such as perforins, granzymes, alarmins, TNFs, and INFs) allows efficient regulation of the degree of broad/global activation of a lymphocyte when exposed to activating antigens. In embodiments, GFP is used to compare the degrees of activation between manufacturing lots and ensure consistency for therapeutic development.

In embodiments, BioNV/exosomes are formed by obtaining iPSCs (hypo-iPSC). In embodiments, the hypo-iPSCs are characterized by a B2M−/−, CIITA−/−, CD47+/+, PD1−/− plasma membrane profile and may be used to generate the present BioNV/exosomes. Hypo-BioNV/exosomes can be generated from the parent iPSC cell line via sonication, adaptive focused acoustics technology, French press, extrusion, serial extrusion, cell lysis by detergent, and electroporation, among other methods. In embodiments, serial extrusion is the method used to generate Hypo-BioNV/exosomes. Serial extrusion of iPSCs can produce BioNV/exosomes that are HLA1/HLA2 negative (hypoimmunogenic) with tgCD47+, exhibiting PD1 resistance elimination.

In embodiments, the obtained hypoimmunogenic cells are CD34+, or derived from CD34+ cells, such as human CD34+ cord blood. In embodiments, CD34+ cord blood-derived cell lines may serve as a base cell line for therapeutic BioNV/exosome development, production, and manufacturing for the delivery of gene editing therapeutics. In embodiments, CD34+ cord blood-derived hypo-immunogenic cell line has been experimentally confirmed for the low expression of HLA 1/2 and overexpression of CD47 (Deuse T. et al. “Hypoimmunogenic derivatives of induced pluripotent stem cells evade immune rejection in fully immunocompetent allogeneic recipients.” Nat Biotechnol. 2019; 37(3):252-258).

In embodiments, the hypoimmunogenic cell can include engineered cells using multiple hypoimmunogenic engineering techniques, for example, by Deuse et al., Han et al., Xu et al., Harding et al., and also as described in published U.S. patent applications US20190376045, 20190376045, 20210308183, and 20210292715 to Deuse, US20210161971 to Nagy, US20180141992 to Strominger, and Published European patent application 3693384 to Poirot, each of which is incorporated by reference herein in their entirety (Han X, et al. “Generation of hypoimmunogenic human pluripotent stem cells.” PNAS. Vol. 116, No. 21 2019: pp. 10441-10446. doi: 10.1073/pnas.1902566116.) and (Xu H, et al. “Targeted Disruption of HLA Genes via CRISPR-Cas9 Generates iPSCs with Enhanced Immune Compatibility.” Cell Stem Cell. Vol. 24, No. 4, 2019: pp. 566-578. doi: 10.1016/j.stem.2019.02.005.).

In embodiments, BioNVs/exosomes are derived from cells which have eliminated HLA genes that encode the MHC membrane glycoproteins that confer immune reactions associated with GVHD rejections. The HLA gene clusters can be divided into three categories: 1) the MHC Class I pathway, 2) the MHC Class II pathway, and 3) the MHC Class III pathway. Only the MHC Class I and II pathways express the protein complexes elicit an immune response in GVHD, whereas MHC Class III complexes are not involved in immunization activities.

The elimination of the MHC classes of protein complexes can trigger natural killer cells and macrophages into an active clearance mode where the cells are subsequently destroyed. To avoid this kill mechanism, in embodiments, the addition of a CD47 isoform 2 transmembrane molecular protein tag can be engineered into the cell membrane of the modified cell to avoid natural killer and macrophage-mediated kill responses, for example, as described in Willingham et al., Deuse et al., and Han et al. (Willingham S B, et al. “The CD47-signal regulatory protein alpha (SIRPa) interaction is a therapeutic target for human solid tumors.” PNAS. Vol. 109, No. 17, 2012: pp. 6662-7. doi: 10.1073/pnas.1121623109.).

In embodiments, cells can be engineered to use additional mechanisms to prevent these responses such as those described in 1) the CD24 transmembrane molecular protein tags (for example as performed in Zhao et al.) (Zhao W, et al. “Strategies for Genetically Engineering Hypoimmunogenic Universal Pluripotent Stem Cells.” iScience. Vol. 23, No. 6, 2020:101162. doi: 10.1016/j.isci.2020.101162.), 2) the membrane-bound surfactant protein-D (SP-D) (for example as performed in Jiaravuthisan et al.) (Jiaravuthisan P, et al. “A membrane-type surfactant protein D (SP-D) suppresses macrophage-mediated cytotoxicity in swine endothelial cells.” Transpl Immunol. Vol. 47, 2018: pp. 44-48. doi: 10.1016/j.trim.2018.02.003.), and 3) the molecular PD-L1 tag for prevention of T-cell responses. In embodiments, a BioNV derived from an ‘activated’ cell would encapsulate and/or release perforin and/or granzyme, resulting in targeted cell death. In embodiments, the activated cell would generate perforin and/or granzyme to be packaged into the BioNV. In embodiments, hypoimmunogenic cells that are to be activated would not express PD-L1 to avoid the resultant BioNV from being targeted to PD-1 on T-cells. In embodiments, this reduces the likelihood of releasing perforin and/or granzyme, resulting in unwanted T-cell death. In embodiments, PD-L1 is overexpressed in BioNVs derived from a cell that has not been activated and is not loaded with apoptotic cytokines. In embodiments, hypoimmunogenic cells that are to be activated have PD-L1 downregulated, knocked-out, or otherwise silenced. In embodiments hypoimmunogenic cells that are not to be activated have PD-L1 upregulated, i.e., for BioNVs used for gene editor delivery. In embodiments, CD47 isoform 2, can be engineered into the cell to prevent both macrophage and natural killer-mediated cytotoxicity, because it acts as a “don't eat me” tag through the SIRP-α receptor that is expressed on these cells, among other cells. In embodiments, CD47 can be utilized in genetically engineered iPSCs for immune tolerance to innate immune cells, for example, such as in Chhabra et al., Han et al., and Jaiswal et al. (Chhabra A, et al. “Hematopoietic stem cell transplantation in immunocompetent hosts without radiation or chemotherapy.” Sci Transl Med. Vol. 8, No. 351, 2016: 351ra105. doi: 10.1126/scitranslmed.aae0501.) and (Jaiswal S, et al. “CD47 is upregulated on circulating hematopoietic stem cells and leukemia cells to avoid phagocytosis.” Cell. Vol. 138, No. 2, 2009: pp. 271-85. doi: 10.1016/j.cell.2009.05.046.). In embodiments, cells can be modified as described in U.S. Pat. No. 8,562,997 to Jaiswal, et al., which is incorporated by reference herein in its entirety.

In embodiments, some approaches can be used which do not entirely knock-out all HLA genes, for example, as performed in Xu et al. and Han et al., which only knock-out the HLA genes that are highly associated with an immune response, leaving intact the HLA genes that dampen a macrophage or NK response (e.g., HLA-E, HLA-F, and HLA-G). In embodiments, this approach does not require the addition of a CD47 tag; the modified cell can be engineered to generate BioNVs/exosomes with or without CD47.

In embodiments, methods improve upon the approaches of hypoimmunogenicity of Table 1.

TABLE 1
Three methods of modification of cells using the HLA knockout combined with a CD47 isoform
2 tag and a PD-L1 transmembrane tag (Zhao, et al.) and (Gornalusse GG, et al. “HLA-
E-expressing pluripotent stem cells escape allogeneic responses and lysis by NK cells.”
Nat Biotechnol. Vol. 35, No. 8, 2017: pp. 765-772. doi: 10.1038/nbt.3860.).

| | | | | | | | | | |
|---|---|---|---|---|---|---|---|---|---|
| Source | HLA-A | HLA-B | HLA-C | HLA-E | HLA-F | HLA-G | HLA-II | CD47 | PD-L1 |

| Gornalusse, 2017 | Knockout | + | Knockout | | |
|---|---|---|---|---|---|
| Deuse, 2019 | B2M Knockout | | | CIITA KO | + |
| Xu, 2019 | Knockout | | | CIITA KO | |

| | | | | |
|---|---|---|---|---|
| Han, 2019 | Knockout | CIITA KO | + | + |

In embodiments, developing the allogeneic modified cell involves the removal of MHC Class I and MHC class II protein complexes through the disruption of certain HLA genes, or a B2M knockout, followed by knocking out the CIITA gene. In embodiments, the knockouts can be performed using CRISPR gene editing approaches, due to their rapid mechanism of action. In embodiments, the knockouts are performed using Zinc Finger Nucleases (ZFNs) and/or TALENS. In embodiments, Cre/Lox recombinase systems are used to generate the modified cell. In embodiments, RNA silencing (RNAi, shRNA, microRNA, CRISPR Cas13a-d, etc.) is used to generate the modified cell.

In embodiments, developing the allogeneic modified cell includes the Harding et al. methods of creating allogenicity that is distinct from the above methods (Harding et al., “Induction of long-term allogeneic cell acceptance and formation of immune privileged tissue in immunocompetent hosts.” BioRxiv 716571 [Preprint], Jul. 30, 2019. doi:10.1101/716571.). In embodiments, in lieu of deleting the MHC class I/II genes and running the risk of preventing long-term acceptance by the recipient, the Harding et al. method includes an alternate approach based on immune escape mechanisms that occurs in nature. In embodiments, the method relies on the Harding et al. biomimicry based on the horizontally transmitted cancer devil facial tumor disease (DFTD) type 2 that is predominant in Tasmanian devils. In embodiments, developing the allogeneic modified cell includes over-expression of the immunomodulatory proteins CCL21, PD-L1, FasL, SerpinB9, H2-M3, CD47, CD200, and/or MFG-E8 to protect cell derivatives from long-term immune rejection in mice (and humans), without the deletion of MHC class I/II proteins. In embodiments, the modified cell expresses one or more of the proteins shown in Table 2, including any splice variant and/or isoform of any of the indicated proteins (e.g., CD200 splice variants). In embodiments, this system can be used to interfere with the activity of antigen presenting cells (APCs), macrophages, natural killer cells, and T-lymphocytes. In embodiments, the modified cell lines can also contain the safe-cell system developed by Liang et al. 2018, where cell division genes are linked to a suicide gene to prevent runaway teratomas leading to cancers (Liang Q, et al. “Linking a cell-division gene and a suicide gene to define and improve cell therapy safety.” Nature. Vol. 563, No. 7733, 2018: pp. 701-704. doi: 10.1038/s41586-018-0733-7.).

In embodiments, methods improve upon the approaches of hypoimmunogenicity of Table 2.

TABLE 2
Expression or increased expression of illustrative
proteins for creating allogenic modified cells.

| | | Illustrative |
|---|---|---|
| | | Immunomodulatory |
| Gene | Type of protein | Function |
| Ccl2 | Chemokine | Recruitment of activated DCs |
| PD-L1 | Check Point Inhibitor | Shuts Down Effector T-cell |
| (non-activated | | Responses |
| cells and/or | | |
| BioNVs) | | |
| FASL | Transmembrane Signal | Induces T-cell Apoptosis |
| H2-M3 | Antigen Presentation - | Inhibits NK cells |
| | MHC-like | |
| SERPINB9 | Inhibitory Chemokine | Blocks granzyme B function |
| | (Serine Protease | |
| | Inhibitor) | |
| Cd47 | Transmembrane | Blocks phagocytosis |
| | SIRP-αR ligand | |
| Cd200 | Type 1 Membrane | Prevents macrophage and |
| | Glycoprotein | granulocyte activation |
| Mfg-e8 | Chemokine (attractant) | Macrophage recruitment to |
| | | anti-inflammatory profile |
| | | site |

In embodiments, obtaining cells from which BioNVs/exosomes are derived includes cells that are engineered to have knock-outs of one or more of HLA-A, HLA-B, HLA-C, HLA-E, HLA-G, HLA-F, CIITA, IL-6, IL-4, IL-10, IL-16, TRAC, TRBC, and/or any combination thereof; and knock-ins of one or more of CCL2, PD-L1 (in BioNVs derived from non-activated cell sources), CTLA-4, H2-M3, CD24, CD47 (minus the 3′ UTR region or an alternate 3′ UTR region that does not contain binding sites for the inhibitory microRNAs), MFG-E8, CD200, and/or any combination thereof.

In embodiments, BioNVs are generated from a modified cell with one or more of the modifications of Table 3.

TABLE 3
Illustrative engineered cell expression profile for BioNV
formation for human use (Fife B T and Bluestone J A. “Control
of peripheral T-cell tolerance and autoimmunity via the CTLA-4
and PD-1 pathways.” Immunol Rev. Vol. 224, 2008:
pp. 166-82. doi: 10.1111/j.1600-065X.2008.00662.x.) and (Rong
Z, et al. “An effective approach to prevent immune rejection
of human ESC-derived allografts.” Cell Stem Cell.
Vol. 14, No. 1 2014: pp. 121-30. doi: 10.1016/j.stem.2013.11.014.).

| | Knock-ins (inducible or constitutive |
|---|---|
| Knockouts | over-expression) |
| HLA-A | CCL2 |
| HLA-B | PD-L1 (non-activated cells and/or BioNVs) |
| | and/or CTLA-4 (Fife et al. and Rong et al.) |
| HLA-C | H2-M3 |
| HLA-E or HLA-G | CD24 or CD47 or CD47 tethered to CD24 |
| (but not both) | |
| HLA-F | MFEG8 |
| CIITA | CD200 for non-immunomodulatory functions |
| IL-6 | only (such as liver cell regeneration). |
| | These cells will not contain a |
| | CD47 tag. |
| IL-4, IL-10, or IL-16 | SerpinB9 |
| (optional) | (where granzyme is not used) |
| TRAC | |
| TRBC | |
| SerpinB9 | |
| (where granzyme | |
| is used) | |

In embodiments, inactivation/activation of genes is controlled by inducible promoters throughout the differentiation, activation, and manufacturing process for BioNVs/exosomes. In embodiments, disruption of MHC, TCR, and CRS genes produce allogeneic iPSCs which are −/− CRS and −/− TCR, leading them to have plasma membranes which exhibit hypoimmunogenic properties upon infusion into a subject. CRS genes implicated in the pathogenesis of CRS include IL-6, IL-10, IFN-γ, monocyte chemoattractant protein 1 (MCP-1), granulocyte-macrophage colony-stimulating factor (GM-CSF), among other cytokines, including tumor necrosis factor (TNF), IL-1, IL-2, IL-2-receptor-α, and IL-8. In embodiments, one or more of these genes is inactivated, e.g., in a cell from which the BioNVs/exosomes are derived.

In embodiments, the obtained hypoimmunogenic cell to generate the BioNVs/exosomes is a metabolically geared (e.g., a specific and differentiated cell lineage) to produce one or more desired therapeutically-relevant biomolecules, or to increase the concentration (or regulate to a desired concentration), therapeutically-relevant biomolecules, so that the regulated cell can then be processed into a BioNV/exosome. In embodiments, the cell

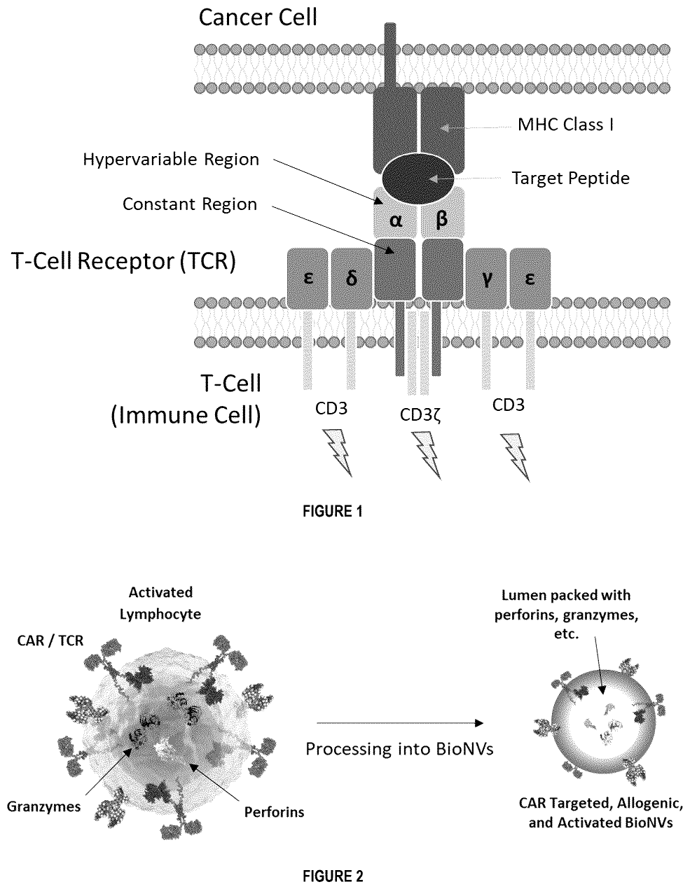

In embodiments, the broad or global regulation of cells results in the expression of multiple genes to produce cytokines, chemokines, regulatory nucleic acids, among other therapeutically-relevant biomolecules that gear a cell into an ‘activated’ state, thereby enhancing the cell to fulfill its metabolically destined purpose/phenotype. In embodiments, controlled expression of the one or more therapeutically-relevant biomolecules in cells can recapitulate, for example, T-lymphocytes entering the activated state that occurs the T cell receptor (TCR) engages an Antigen Presenting Cell (APC) (FIG. 1). In embodiments, controlled expression of the one or more therapeutically-relevant biomolecules in cells can recapitulate the interaction(s) between the antigen peptide that is presented in the Major Histocompatibility Complex (MHC) and the TCR of the T cell, which initiates conformational changes in the TCR that trigger intercellular signaling cascades and mass gene expression of cytokines including perforins, granzymes, alarmins, interleukins, and interferons to name a few (FIG. 1). Typically, a cytokine ‘activates’ a cell into a specialized mode that allows the cell to clear the antigen from the host and recruit immune cells to the site of infection to aid in the clearance of the antigen/infected cells and repair of the surrounding tissue. In embodiments, these cellular processes occur without supplying an exogenous cytokine, which can lead to deleterious effects in the cell line, such as exhaustion, competing signaling pathways, etc. In embodiments, the activated state of the T-cell, for example, can be harnessed in a BioNV/exosome through processing methods in the form of capturing the cytokines or transmembrane ligands in its lumen and/or membrane, respectively (FIG. 2).

In embodiment, broad or global activation of cells (e.g., T-cells, Natural Killer cells) can be achieved outside the host in culture (in vitro). In embodiments, the activated cells can then be expanded and processed into BioNVs/exosomes that act like miniature T-cells, without the complex genetic baggage of the entire cell. The broad regulation of T-cells in vitro can be achieved by numerous methods described herein.

In embodiments, activating the hypoimmunogenic cell comprises activation of a TCR/CD3 receptor complex by a protein antigen (in the case of T-cells). In embodiments, activating the hypoimmunogenic cell comprises activation of a TCR/CD3 receptor complex by a small molecule. In embodiments, small molecule stimulation used for cellular activation by a TCR/CD3 receptor can include phorbol esters. In embodiments, activating the hypoimmunogenic cell comprises activation of a TCR/CD3 receptor complex by a viral antigen. In embodiments, the viral antigen can be a recombinant viral protein, such as an extracellular viral antigen (e.g., glycoprotein or spike protein) or intracellular viral antigen, etc.

In embodiments, activating the hypoimmunogenic cell comprises activation of a calcium dependent channel. In embodiments, calcium dependent channels include calcium release-activated channels (CRACs) which are activated by an increase in Ca2+ concentration, for example, by supply into the culture media. In embodiments, potassium channels (K channels) or calcium channels (Ca channels) can be used, or overexpressed in cells, with the cognate inorganic component supplied in the media to activate cells. In embodiments, prolonged rise in intracellular calcium or potassium is a signal to mimic lymphocyte activation by antigens or mitogens.

In embodiments, activating the hypoimmunogenic cell comprises activation of an LFA-1 integrin receptor. In embodiments, LFA-1 integrin receptor activation can include supplying activating ligand peptides, including ICAM1, ICAM2 (CD102), ICAM3 (CD50), ICAM4, ICAM5, JAM-A, and/or portions thereof.

In embodiments, activating the hypoimmunogenic cell comprises activation of a CD28 receptor. In embodiments, activation by CD28 can be performed by supplying one or more ligand peptides for CD28, including CD80, CD86, GRAP2 and Grb2. In embodiments, activation by CD28 is performed in cell subsets that are not memory T cells.

In embodiments, activation by IL-12 can be performed by supplying one or more of recombinant human IL-12. In more detail, NK cells constitutively express perforins and granzymes. Secretory lysosomes (˜500 nm in diameter) containing the perforin and granzymes are held at a constant concentration within the cell, allowing the Natural Killer cell to be primed/ready for cytotoxic activity, without delay. CD8+ Killer T-cells (and their cellular subsets) also contain low to mid-level concentrations of perforins and granzymes in lytic granules. However, in other T-cells (CD4+/CD8+ memory and helper T-cells for example), perforins and granzymes within lytic granules are generally low or absent, resulting in delayed expression and cytotoxic action that could take several days to a week to accumulate enough lytic granules to initiate its cytotoxic phenotype. This delay is a regulatory and buffered feature among the differing cell types to prevent runaway reactions from occurring. By analogy, NK and Killer T-cells are like a garden hose loaded with water. When someone turns on the faucet, water immediately comes out. On the other hand, other T-cells are like a garden hose that is empty. When the faucet is turned on it takes time for the water to come out—a delayed reaction. From an activation perspective for manufacturing, using NK and/or Killer T-cells derived from iPSCs are ideal because they are already primed with secretory lysosomes and lytic granules, respectively, and can be stimulated/activated in a manner to rapidly produce additional perforins and granzymes, if necessary Other T-cells (or other cells such as neutrophils) can still be used as source for cytotoxic proteins, but the time to express them may be extended. The former cell types will reduce costs.

In embodiments, activating the hypoimmunogenic cell comprises activation of an IL-2 receptor and an IL-12 receptor. In embodiments, activation by IL-2 can be performed by supplying one or more of recombinant human IL-2. In embodiments, activation by IL-12 can be performed by supplying one or more of recombinant human IL-12. IL-2 and IL-12 increase the accumulation of perforin and granzyme B mRNA in NK and Killer T-cells. It is likely that the mRNA transcripts for perforin and granzyme(s) form a low level ‘primed and sustainable pool’ (the water in the garden hose) in the cytoplasm and act as the source for rapid deployment during activation. IL-2 and/or IL-12 are likely better candidates for cellular activation in vitro than other sources or modes of action such as antigen receptor engagement on other cells, or antigen stimulation on surface receptors because these paths have been shown to only cause an increase in granzyme(s). The production of both perforins and granzymes is therefore ideal and can be achieved with IL-2 and IL-12 stimulation through their respective receptors. Further, IL-2 has been shown to stabilize perforin and granzyme mRNA stability in the cytoplasm. Therefore, IL-2 is likely the preferable activating cytokine over IL-12, because it helps to maintain these cytoplasmic mRNAs reservoirs (IL-2 keeps the water in the hose) (REF—PMID: 8103068).

In embodiments, the cytokine INF-α can also be used be used to activate the JAK1/TYK2 to STAT pathway, via the INFAR1 and/or INFAR2 receptors, at least in T-cells, for the production of perforins and granzyme(s).

In embodiments, activating the hypoimmunogenic cell, such as a Natural Killer cell (or T-cell if the receptor(s) listed below is relevant to T-cell activation), comprises activation of a receptor (either individually or in combination to allow synergistic activation responses) such as CD2, CD3, CD16, CD28, CD314 (NKG2D), CD335, B7-H6, CD158d, IL-2R, IL-12R, DNAM-1, CD2, CD44, CD137, CX3CR1, CD27, CD160, 2B4 but not limited to these sensory receptors. CD16 can be activated via binding to the Fc region of antibodies (anti-S2 IgG). CD134, CD335, B7-H6 can be activated in combination with ICAM-1 combined with the Fc region of antibodies. CD314 can be activated by ULBP1, MICA, MICB, or H60. CD158d can be activated to trigger proinflammatory molecules via HLA-G. IL-2R can be activated by molecular IL-2. IL-12R can be activated by molecular IL-12. DNAM-1 can be activated by CD155 or CD112 or NKp30. CD2 can be activated by LFA3. CD44 can be activated by hyaluronic acid, hyaluronan, osteopontin, collagens, or matrix metalloproteinases. CD28 homolog can be activated by B7H7 protein ligand. CD137 can be activated by itself and members of the Tumor Necrosis Factor Receptor family of proteins. CX3CR1 can be activated by CX3CL1. CD27 can be activated by CD70. CD160 can be activated by HLA-C. 2B4 can be activated by CD48. Each type of activation can lead to differing levels of cytotoxic biomolecules. The activation of each (or any) combination above can occur by the addition of the each of the following ligands (or any other relevant ligand) respectively, either in soluble form (partial peptide, domain or whole protein), anchored in a membrane (partial peptide, domain or whole protein), a target cell membrane constituent (partial peptide, domain or whole protein), or cross-linked to a solid phase such as, but not limited to, sephadex or magnetic beads (as a partial peptide, domain or whole protein).

In embodiments, the activation of some Natural Killer receptors (or T-cell if the receptor(s) listed below is relevant to T-cell activation), does not lead to degranulation or granule polarization to the IS. These receptors include, but are not limited to, i) 2B4 individually, ii) CD134 individually, iii) CD134 in combination with 2B4 and one of either inhibitory receptor KIR or CD94, iv) LFA-1 in combination with one of either inhibitory receptor KIR or CD94, v) CD16 in combination with one of either inhibitory receptor KIR or CD94. The activation of each (or any) combination above can occur by the addition of the each (or any other relevant ligand) of the following ligands respectively, either in soluble form (partial peptide, domain or whole protein), anchored in a membrane (partial peptide, domain or whole protein), a target cell membrane constituent (partial peptide, domain or whole protein), or cross-linked to a solid phase such as, but not limited to, sephadex or magnetic beads (as a partial peptide, domain or whole protein), i) CD48, ii) ULBP1, iii) HLA-E, iv) HLA-C in combination with HLA-E, or v) HLA-E.

In embodiments, the activation of some Natural Killer receptors (or T-cell if the receptor(s) listed below is relevant to T-cell activation), leads to degranulation. Immediately prior to degranulation, perforins and granzymes may (or may not) undergo post-translational modifications that enhance their activities. It may be favorable to activate cells along the degranulation pathway to achieve maximum effectiveness of cytotoxic proteins. These receptors include but are not limited to, i) CD16 in combination with LFA-1 and one of either inhibitory receptor KIR or CD94, ii) CD134 in combination with 2B4, iii) CD16 individually. The activation of each (or any) combination above can occur by the addition of the each of the following ligands (or any other relevant ligand) respectively, either in soluble form (partial peptide, domain or whole protein), anchored in a membrane (partial peptide, domain or whole protein), a target cell membrane constituent (partial peptide, domain or whole protein), or cross-linked to a solid phase such as, but not limited to, sephadex or magnetic beads (as a partial peptide, domain or whole protein), i) HLA-C in combination with HLA-E, ii) ULBP1 in combination with CD48, iii) anti-S2 IgG.

In embodiments, receptors that when activated, lead to the polarization of lytic granules may not (or may be) be favorable, as their localization to the cell membrane may minimize their packaging in to BioNVs, but may maximize their packaging into secreted EVs (exosomes and microsomes), due to differences in extrusion manufacturing (BioNVs) vs. cellular exocytosis processing (EVs). Some receptors, that induce the polarization of lytic granules to the IS include but are not limited to, i) CD16 in combination with LFA-1 (also leads to degranulation), ii) LFA-1 individually, iii) CD134 in combination with LFA-1 and 2B4 (also leads to degranulation). The activation of each (or any) combination above can occur by the addition of the each of the following ligands (or any other relevant ligand) respectively, either in soluble form (partial peptide, domain or whole protein), anchored in a membrane (partial peptide, domain or whole protein), a target cell membrane constituent (partial peptide, domain or whole protein), or cross-linked to a solid phase such as, but not limited to, sephadex or magnetic beads (as a partial peptide, domain or whole protein), i) anti-S2 IgG in combination with ICAM-1, ii) ICAM-1, iii) ULBP1 in combination with CD48 and ICAM-1.

In embodiments, activating the hypoimmunogenic cell comprises activation of a SLAMF1(CD150) receptor. In embodiments, activation of cells via the SLAMF1(CD150) receptor can be performed with one or more SLAM-associated proteins. In embodiments, said activation by the SLAMF1(CD150) receptor is by an interaction with a measles virus. In embodiments, said activation by the SLAMF1(CD150) receptor is by an interaction with a Gram negative bacteria.

In embodiments, activating the hypoimmunogenic cell comprises activation of an IFNγ receptor. In embodiments, activation by an IFNγ receptor can include any Toll-like Receptor (TLR) superfamily member, and can be performed by supplying one or more TLR receptor agonists, for example, IFN-γ, lipoteichoic acid (LTA), lipopolysaccharide (LPS), gardiquimod, R848, CpG, Pam3CSK4, etc.

In embodiments, activating the hypoimmunogenic cell comprises activation of a CD4 receptor by one or more viruses. In embodiments, activating viruses include (but are not limited to), the two distantly related Arena Viruses; Pichinde Virus and Lymphocytic Choriomeningitis Virus. Pichinde Virus and Lymphocytic Choriomeningitis Virus have been shown to induce tumor-specific CTL responses up to 50% of the circulating CD8+ T cell pool. In embodiments, ex vivo activation of CD8+ T cells can increase the levels alarmin(s) (such as, without limitation, IL-1α, IL-33, and IL-17), which in turn would be packaged (along with other anti-cancer biomolecules, e.g., perforins and granzymes) into the BioNVs/exosomes after their derivation from the virally activated cell. In embodiments, alarmins can increase the elicit potent cytotoxic effector T lymphocyte (CTLeff) responses in treating diseases. In embodiments, the response generating from administering the BioNVs/exosomes herein would be localized at the site of the disease (e.g., tumor, infected cell, etc.) to where the BioNVs/exosomes that carry the alarmins are specifically targeted.

In embodiments, activation of the expression of cytotoxic biomolecules such as perforins and granzyme(s) can be accomplished by targeting cytoplasmic signaling molecules (such as kinases, phosphatases, GTPases. ATPases, etc.) that are part of the signaling pathway, downstream from membrane associated receptors and factors. In embodiments, an example of such an approach in T-cells could involve a small molecule that activates STATs, Blimp1, Tbet, Runx3, thPOK (by blocking Runx3 or eome inhibition).

In similar embodiments, PKC (PKCtheta), a serine- and threonine-specific protein kinase(s) and transduction signaling molecule central to T-cell and NK cellular activation can be activated by calcium and the second messenger diacylglycerol. Small molecules can also be added to the cell that cross the cellular membrane and activate PKC, resulting in the production of perforins and granzymes. There are a number of commercially available small molecules that can activate PKC. These include (but are not limited to):

Bryostatin 1: Potent agonist with binding affinities to classical and novel PKC isozyme subgroups (REF—PMID: 16834754).

Ingenol-3-angelate (I3A, PEP005): Broad PKC activator. Proapoptotic and immunostimulatory effects Likely promotes degranulation processes. (REF—PMID: 22069553).

Phorbol 12-myristate 13-acetate: Reversible PKC activator. Activates Ca2+-ATPase (REF—PMID: 3478199).

Prostratin: PKC and NF-κB activator. Synergizes with calcium/calcineurin signaling. Potent synergistic activator of perforin and granzyme(s). (REF—PMID: 24204950).

SC-9, SC-10: PKC activator that induces PKC phosphorylation. PKC inhibition can be restored with SC-10 Dual system that can activate cells, then reduce activated state once perforin and granzyme(s) optimal levels are reached. (REF—PMID: 1414485).

Phorbol-12,13-dibutyrate (PDBu): Increases kinase phosphorylation. Induces contraction of vascular smooth muscle. Release of mobilized Ca2+ likely leads to perforin activation in a preloaded state (REF—PMID: 19632318)

Other PKC activators can be found at Santa Cruz.

In similar embodiments, Crk, an adapter protein that binds to some tyrosine-phosphorylated proteins, and acts as a key regulator of activation in NK cells, can be target for the activation or release of secondary inhibitory related functionalities to achieve perforin and granzyme expression. MHC-I inhibitory receptors block activation signals resulting in the phosphorylation of Crk and its subsequent association with c-Abl (REF—PMID: 18835194). HLA-E induces Crk phosphorylation in NKG2A+NK cells (REF—PMID: 22464172). Because Crk is required for CD16 activation (as mentioned above), prevention of inhibitory mechanisms by small molecule interruption, in combination with CD16 receptor activator can be an alternate way to activate NK cells to produce perforins and granzymes into secretory lysosomes. Also, a small molecule blocker of Crk phosphorylation (1-(2,6-Dichlorophenyl)-1,5-dihydro-6-((4-(2-hydroxyethoxy)phenyl)methyl)-3-(1-methylethyl)-4H-pyrazolo[3,4-d]pyrimidin-4-one) is an example of a small molecule that inhibits Crk phosphorylation in combination with a small molecule activator can be an alternate way to activate NK cells. In embodiments, the Crk phosphorylation site can be mutated to prevent inhibition. The mutation can be transient or stable in the cell. In such a mutant, a small activating molecule can be used to activate the cell through Crk. Similar strategies can be used at other points of signal transduction pathways that lead to NK activation resulting in the production of perforins and granzymes. As mentioned above, some of the points of activation (targeted proteins in the signaling pathway(s)) may result in cross talk between signaling pathways that may cause varying degrees of calcium dependent perforin activation and lor perforin and granzyme polarization within the cell. As such, depending on the desired state of each protein prior to BioNV manufacturing processes, the type of activating molecule will need to be selected to suit those purposes. For example, perforin in its folded and Ca2+ unbound state may be desirable over perforin bound to Ca2+. The latter may lead to unintended off-target effects on healthy tissues in the context of being packaged in a BioNV. Thus, small molecules that activate the expression of native perforin without inducing calcium release will be desirable.

In embodiments, the activation of transcription factors that lead to the activation of perforins and granzymes may also be used. In this approach, a transcription factor that targets only perforin promoters for increase in expression may be desirable if the intention is to increase the amount of perforin to levels higher than granzyme. In another approach a transcription factor that targets only granzyme promoters for increase in its expression may be desirable if the intention is to increase the amount of granzyme to levels higher than perforin. In another approach each transcription factor can be activated simultaneously to produce equivalent levels of each protein. Small molecules can be used to activate the transcription factors. Proteins designed/engineered (transiently or stably expressed) to interact with transcription factors or transcription factor genes, can also be used to activate transcription factors. Examples of transcription factors that positively regulate the expression of perforins and granzyme include eomes and NF-κB (but not limited to these). Eomes can be activated by CRISPR activating plasmids (REF—EOMES CRISPR). NF-κB can be activated by the small molecule(s) 4-Hydroxyquinazoline; 4-Quinazolinol.

In embodiments, CRISPR activation and inhibition systems (CRISPRa/i) can be used to directly activate or repress perforin and/or granzyme gene expression function, independent of upstream signaling. In such a system, the CRISPRa/i can be engineered transiently (episomally) or stably integrated into the desired iPSC cell that can be differentiated into the desired cell type (T-cell or NK for example). After differentiation, in a stably integrated cell line, the CRISPRa can be activated using a drug inducible promoter such as tetracycline (but not limited to tetracycline or tetracycline-based promoters). Once induced, CRISPRa and its respective gRNAs that target the promoter regions of perforin and granzyme are expressed. The CRISPRa gRNAs bind to and express perforin and granzyme. The advantage of this system over the activation approaches above is controlled expression of perforin and granzymes without placing the cell in an activated state. This system can be used to control the expression of other cytotoxic proteins or proteins of interest with therapeutic value, that can be packaged into the lumen of the BioNV. The expression of the desired proteins can then be turned off using CRISPRi plus gRNAs targeting the same regions of the protomers.

In embodiments, perforin and granzyme genes can be engineered into an iPSC and contain regulatory properties embedded in the promoters to allow direct and controlled expression using, for example, a drug inducible or CRISPRa/i or microRNA regulated systems. In this approach, the iPSCs containing the engineered cytotoxic genes can be differentiated into any desired cell type such as monocytes, macrophage, fibroblast, kidney cell, hepatocytes etc. This approach will allow a cell to express perforins and granzymes (and potentially other engineered cytotoxic proteins if necessary or applicable) that it cannot otherwise naturally express (nor can they be activated to express these proteins). The purpose of this approach is to allow the expression of such proteins while exploiting cellular properties that are different from lytic granule producing cells (such as Natural Killer cells, T-cells, neutrophils, etc.). Such exploitable cellular properties include surface markers that enhance tissue tropisms, barrier crossing phenotypes, and higher precision in cellular or tissue targeting that may be lacking in Natural Killer cells, T-cells, neutrophils, etc.)

In embodiments, activating the hypoimmunogenic cell comprises activation by an engineered, non-native biomolecule selected from one or more of a soluble peptide, a chimeric antigen receptor, a small molecule decoy, a small molecule ligand, a designer nucleic acid ligand, a carbohydrate ligand, a viral ligand, a chimeric biomolecular ligand, a fusion protein, an antibody, and an antibody format molecule. In embodiments, the T-cell (or desired cell) can be engineered to include a CAR, where the intercellular domain of the CAR construct can be developed to broadly activate the cell through its signaling domains. For example, in embodiments, 4-1BB can be engineered into a traditional CD3 intercellular activation domain(s) to broaden and sustain T-cell activation through activation of noncanonical NF-kB pathways (and subsequent pathways). In embodiments, the cell is activated by the biomarker antigen that the extracellular domain of the CAR construct has been engineered to recognize through its scFv recognition domain(s). In embodiments, this broad activation can occur through biomarker recognition by different types of extracellular CAR domains such as scFv, V_{H}H nanobody, V_{NAR}, affilins (or other affinity targeted biochemical ligands), or CERs,) or through TCR ligands and components.

In embodiments, activating the hypoimmunogenic cell comprises activation by an inorganic compound (e.g., iron, calcium, potassium, tin, mercury, arsenic, pyrophosphates, etc.).

In embodiments, activating the hypoimmunogenic cell comprises activation by expression, overexpression, or increased activity of a transcription factor (e.g., signal transducer and activator of transcription (STATs), T-bet, Blimp1, NF-kB, MAPK, etc.). In embodiments, the cell is activated by expression and/or overexpression of a constitutively active form of an intracellular signaling pathway, such as a constitutively active STAT5 molecule to drive signal transduction.

In embodiments, the CAR is activated prior to BioNV/exosome formation. In embodiments, the CAR is activated via its target, through another receptor, and/or a virus. In embodiments, the CAR construct typically contains 1^{st}, 2^{nd}, 3^{rd}, or 4^{th} generation intracellular signaling modalities that have been designed to activate T/NK/macrophage cells (among other cell types) into an active mode (metabolically geared) that results in the expression of various biomolecules such as alarmins, interferons, granzymes, perforins, etc. However, in embodiments, the degree of activation is regulated through a different receptor that can result in a low-level activation of cytokine that is more directed or more targeted for a specific purpose. In embodiments, the modified cell can be activated by IL-15 alone to pack the BioNVs/exosomes with this cytokine to recruit naturally occurring lymphocytes to the tumor site.

In embodiments, the CAR construct (extracellular region) is activated by its antigen (e.g., a soluble version of the antigen biomarker that it is targeting). In embodiments, the antigen can be attached to magnetic beads and added to the cell culture where the magnetic beads can then be taken out of the culture after activation, or, the cells can be run through a chromatography column that contains bound antigen, thereby becoming activated as they flow through the column.

In embodiments, the CAR construct can be activated via the appropriate biomarker “antigen,” or portion thereof, that is recognized by the CAR, which could include, but is not limited to, a binding moiety, epitope, or peptide of an scFv, VERR/viral ligand, V_{H}H nanobody, V_{NAR}, affilins (or other affinity targeted biochemical ligands), CER, or TCR ligand or components. In embodiments, the antigen can be the targeted biomarker or an activating virus, or protein(s) of an activating virus. In embodiments, the antigen activation occurs by binding the antigen on a chromatography column, followed by passing the modified cells over the column to be captured by the antigen via CAR-antigen interaction. In embodiments, the CAR-modified cells are lymphocytes which can then be eluted from the column, resulting in purified, activated lymphocytes. In embodiments, activation by antigen involves the addition of low levels of antigen into the media of the modified cell. In embodiments, after activation (through the antigen-second generation or antigen-third generation CAR interaction), the cells are treated by the methods described herein to create the BioNVs/exosomes. In embodiments, the BioNVs/exosomes contain the second-generation (or 3rd generation) CARs on its surface, and the essential lymphocyte activating proteins (lymphocyte anti-cancer cell or anti-defective cell cytokine repertoire), which can include, but are not limited to perforin and granzyme B.

Generally, activating with antigen can cause caveats due to issues with separating the biomarker antigen from modified cell CAR receptors. In embodiments, several viruses can be added or exposed to the modified cell (e.g., T cells), to activate them outside of the CAR receptor. In embodiments, antibodies can be attached to a piece of iron or magnetic nanoparticles (iron oxide) and magnetism can be used to separate the virus from the cells after activation to remove the virus.

In embodiments, each “natural” activation mode (e.g., calcium/potassium dependent channel, LFA-1 integrin receptor, IL-2 receptor, SLAMF1(CD150) receptor, IFNγ receptor, TLR receptor, CD4 receptor, viruses, etc.) can be supplemented by engineered versions of activating soluble peptides, CARs, small molecule decoys, small molecule ligands, designer nucleic acid ligands, carbohydrate ligands, viral ligands, chimeric biomolecular ligands or fusion proteins, antibodies, antibody format molecules, and/or combinations thereof. In embodiments, activation is performed by at least one, at least two, at least three, or at least four or more different modes of activation simultaneously. In embodiments, any one or more of the activation modes will result in broad and/or global T cell activation sufficient to produce the minimal levels of therapeutically relevant biomolecules (such as the cytokines described herein) that can be captured from the activated and expanded T cells for processing into the lumen of BioNVs/exosomes.

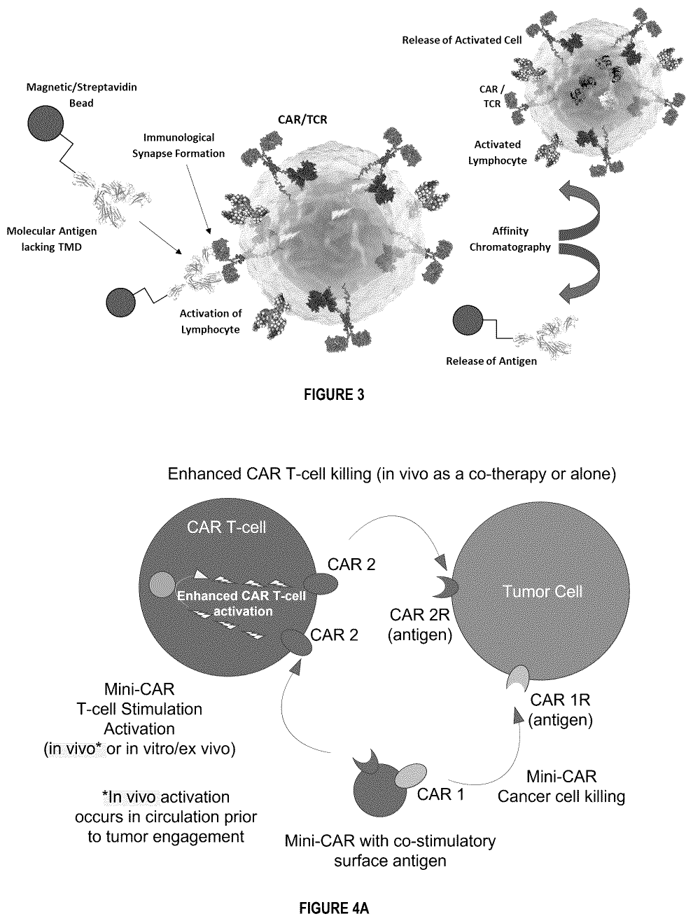

In embodiments, in addition to the pathways of activation, the methods of broad or global cellular regulation can include the conjugation of antigen(s), among other activating molecules, to magnetic or streptavidin-biotin beads to ensure antigen separation from the cell activation receptors (FIG. 3). For example, in embodiments, a biomarker antigen that forms an immunological synapse (IS) with a CAR construct can be conjugated to magnetic or streptavidin-biotin beads and then added to the CAR containing cells in vitro to activate the cells. In embodiments, once activated, the biomarker antigen(s) can be mechanically removed from the cell suspension.

In embodiments, activating the hypoimmunogenic cell comprises activation at the DNA level by one or more of a transposase-based method, Cre/Lox-based method, endonuclease-based method, homologous recombination (HR)-based method, non-homologous end joining (NEHJ)-based method, microhomology-mediated end-joining (MMEJ)-based method, homology-mediated end joining (HMEJ)-based method, small RNA, or a combination thereof. In embodiments, the small RNA comprises one or more of a guide RNA (gRNA), tracer RNA (tracrRNA), micro RNA (miRNA), RNA inference (RNAi), small interference RNA (siRNA), duplex RNA, Piwi-interacting RNA (piRNA), small nuclear RNA (snRNA), small nucleolar RNA (snoRNA), antisense oligonucleotide (ASO), locked nucleic acid (LNA), splice switching oligonucleotide (SSO), tRNA, complementary messenger RNA, repeat associated small interfering RNA (rasiRNA), and small non-coding RNA.

In embodiments, activating the hypoimmunogenic cell comprises activation at the RNA level by one or more guide RNA (gRNA), tracer RNA (tracrRNA), micro RNA (miRNA), RNA inference (RNAi), small interference RNA (siRNA), duplex RNA, Piwi-interacting RNA (piRNA), small nuclear RNA (snRNA), small nucleolar RNA (snoRNA), antisense oligonucleotide (ASO), locked nucleic acid (LNA), splice switching oligonucleotide (SSO), tRNA, complementary messenger RNA, repeat associated small interfering RNA (rasiRNA), endonuclease, small non-coding RNA, IRES element, or a combination thereof.

In embodiments, the cell is activated by one or more RNA-guided endonucleases. In embodiments, RNA-guided endonucleases can be used to inactivate (e.g., cause a knock-out mutation) and/or activate gene expression (e.g., knock-out a gene repressor) for purposes of metabolically tuning a cell or otherwise activating a signaling pathway that results in therapeutically relevant biomolecules. In embodiments, activating the hypoimmunogenic cell comprises activation by an endogenous promoter region and/or enhancer region, for example, by knock-in of a constituently active promoter/enhancer element upstream of a gene of interest. In embodiments, activating the hypoimmunogenic cell comprises activation by stably integrating a genetic element. In embodiments, activating the hypoimmunogenic cell comprises activation by transient expression of a genetic element.

In embodiments, the above examples of activation (e.g., calcium/potassium dependent channel, LFA-1 integrin receptor, IL-2 receptor, SLAMF1(CD150) receptor, IFNγ receptor, TLR receptor, CD4 receptor, and viruses) relate to T cells and subsets of T-cells (e.g., T-helper cells, T-memory cells, etc.) and NK cells. However, in embodiments, the activation of cells through surface receptors with the goal to produce therapeutically relevant biomolecules to be packaged in the lumen of BioNVs/exosomes can occur in any other cell type that can be applicable to a target disease. For example, in embodiments, the broad or global regulation of macrophages can produce different inflammatory and/or anti-antigen directed cytokines at differing concentrations depending on the activation of surface receptors. In embodiments, using alternate cell types can make use of phenotypes related to cell membrane proteins have been shown to transfer to the properties of the BioNV/exosome. For example, in embodiments, macrophage cell membrane proteins involved with tissue and barrier crossing events for the entire cell can be captured in the resulting BioNV/exosome; the resultant BioNV/exosome can then be capable of crossing similar barriers, such as the blood brain barrier.

In embodiments, hepatocytes can be regulated so they produce therapeutically relevant biomolecules that can be used in the context of human disease, such as liver diseases. In embodiments, hepatocytes can undergo broad/global regulation to alter the cell surface receptors that are responsible for downstream cytokine-driven growth and repair at each phase of the cell cycle (e.g., regulation for repair pathways can be segmented at different stages of repair and growth: G0, G1, S-Phase). In embodiments, hepatocytes can be engaged (activated and/or genetically engineered) with methods like those described above in a T cell regulation scenario.

In embodiments, activating the hypoimmunogenic cell results in a metabolically altered state of the hypoimmunogenic cell. In embodiments, methods of activation and/or engineering can be applied to other cell types that are involved with either singular or multi-functional pathways that entail the regulation of the cell from a resting state to a metabolically altered state where newly expressed biomolecules have therapeutically relevant value that can be harnessed in a BioNV/exosome, through BioNV/exosome processing methods.

In embodiments, the one or more therapeutically relevant biomolecules is a chemokine, interferon, interleukin, alarmin, lymphokine, perforin, granzyme, granulysin, tumor necrosis factor (TNF), colony-stimulating factor, bone morphogenic protein (BMP), erythropoietin (EPO), granulocyte-stimulating factor (G-CSF), granulocyte macrophage colony-stimulating factor (GM-CSF), gene editing payload, fusion protein, antibody or antibody format, or a combination thereof.

In embodiments, the one or more therapeutically relevant biomolecules comprises a cytokine. In embodiments, a cytokine refers to a broad category of small proteins on the order of about 5 kDa to about 50 kDa that have important functions in cell signaling; cytokines cannot generally cross the lipid bilayer of cells to enter the cytoplasm. In embodiments, cytokine can include chemokines, interferons (IFNα/β/γ), interleukins (ILs), alarmins, lymphokines, and tumor necrosis factors (TNFs), colony-stimulating factors, bone morphogenic protein (BMP), erythropoietin (EPO), granulocyte-stimulating factor (G-CSF), granulocyte macrophage colony-stimulating factor (GM-CSF), or a combination thereof. In embodiments, the one or more therapeutically relevant biomolecules is a pro-inflammatory cytokine. In embodiments, the one or more therapeutically relevant biomolecules is an anti-inflammatory cytokine. In embodiments, the one or more therapeutically relevant biomolecules is a perforin. In embodiments, the one or more therapeutically relevant biomolecules is a granzyme (e.g., granzyme A, B, H, K, and M).

TABLE 4
Illustrative interleukins that can be packaged into BioNVs/exosomes.

| | | Targets | | |
|---|---|---|---|---|
| | Illustrative | Illustrative | Illustrative | |
| Name | Source | Receptors | Cells | Illustrative Function |
| IL-1 | macrophages, | CD121a/IL1R1, | T helper cells | co-stimulation |
| | B cells, | CD121b/IL1R2 | B cells | maturation & proliferation |
| | monocytes, | | NK cells | Activation |
| | dendritic cells | | macrophages, | inflammation, induce acute phase reaction, |
| | | | endothelium | large amounts induce fever |
| IL-2 | Th1-cells | CD25/IL2RA, | Activated T cells | stimulates growth and differentiation of T cell |
| | | CD122/IL2RB, | and B cells, | response. Can be used in clinical trials to raise |
| | | CD132/IL2RG | NK cells, | CD4 counts in HIV positive patients. |
| | | | macrophages, | |
| | | | oligodendrocytes | |
| IL-3 | Activated T helper | CD123/IL3RA, | hematopoietic | differentiation and proliferation of myeloid |
| | cells, | CD131/IL3RB | stem cells | progenitor cells to e.g., erythrocytes, |
| | mast cells, | | | granulocytes |
| | NK cells, | | mast cells | growth and histamine release |
| | endothelium, | | | |
| | eosinophils | | | |
| IL-4 | Th2 cells, naïve | CD124/IL4R, | activated B cells | proliferation and differentiation, IgG and IgE |
| | CD4+ cell, | CD132/IL2RG | | synthesis. Important role in allergic response |
| | memory CD4+ | | | (IgE) |
| | cells, | | T cells | Proliferation |
| | mast cells, | | endothelium | increase expression of vascular cell adhesion |
| | macrophages | | | molecule (VCAM-1) promoting adhesion of |
| | | | | lymphocytes.^{[48]} |
| IL-5 | Th2 cells, | CD125/IL5RA, | eosinophils | Production |
| | mast cells, | CD131/IL3RB | B cells | differentiation, IgA production |
| | eosinophils | | | |
| IL-6 | macrophages, | CD126/IL6RA, | Activated B cells | differentiation into plasma cells |
| | Th2 cells, | CD130/IR6RB | plasma cells | antibody secretion |
| | B cells, | | hematopoietic | Differentiation |
| | astrocytes, | | stem cells | |
| | endothelium | | T cells, others | Induces acute phase reaction, hematopoiesis, |
| | | | | differentiation, inflammation |
| IL-7 | Bone marrow | CD127/IL7RA, | pre/pro-B cell, | differentiation and proliferation of lymphoid |
| | stromal cells and | CD132/IL2RG | pre/pro-T cell, NK | progenitor cells, involved in B, T, and NK cell |
| | thymus stromal | | cells | survival, development, and homeostasis, |
| | cells | | | ↑proinflammatory cytokines |
| IL-8 or | macrophages, | CXCR1/IL8RA, | neutrophils, | Neutrophil chemotaxis |
| CXCL8 | lymphocytes, | CXCR2/IL8RB/ | basophils, | |
| | epithelial cells, | CD128 | lymphocytes | |
| | endothelial cells | | | |
| IL-9 | Th2 cells, | CD129/IL9R | T cells, | Potentiates IgM, IgG, IgE, stimulates mast cells |
| | specifically by | | B cells | |
| | CD4+ helper cells | | | |
| IL-10 | monocytes, | CD210/IL10RA, | macrophages | cytokine production |
| | Th2 cells, | CDW210B/IL10RB | B cells | Activation |
| | CD8+ T cells, | | mast cells | |
| | mast cells, | | Th1 cells | Inhibits Th1 cytokine production |
| | macrophages, | | | (IFN-γ, TNF-β, IL-2) |
| | B cell subset | | Th2 cells | Stimulation |
| IL-11 | bone marrow | IL11RA | bone marrow | acute phase protein production, |
| | stroma | | stroma | osteoclast formation |
| IL-12 | dendritic cells, | CD212/IL12RB1, | Activated T cells, | differentiation into Cytotoxic T cells with IL-2, |
| | B cells, | IR12RB2 | NK cells | ↑IFN-γ, INF-α, ↓ IL-10 |
| | T cells, | | | ↑IFN-γ, TNF-α |
| | macrophages | | | |
| IL-13 | Activated | IL13R | TH2-cells, | Stimulates growth and differentiation of B cells |
| | Th2 cells, | | B cells, | (IgE), inhibits TH1-cells and the production of |
| | mast cells, | | macrophages | macrophage inflammatory cytokines (e.g. IL-1, |
| | NK cells | | | IL-6), ↓ IL-8, IL-10, IL-12 |
| IL-14 | T cells and certain | | activated B cells | controls the growth and proliferation of B cells, |
| | malignant B cells | | | inhibits Ig secretion |
| IL-15 | mononuclear | IL 15RA | T cells, activated B | Induces production of Natural killer cells |
| | phagocytes, | | cells | |
| | macrophages | | | |
| IL-16 | lymphocytes, | CD4 | CD4+ T cells (Th- | CD4+ chemoattractant |
| | epithelial cells, | | cells) | |
| | eosinophils, CD8+ | | | |
| | T cells | | | |
| IL-17 | T helper 17 cells | CDw217/IL17RA, | epithelium, | osteoclastogenesis, angiogenesis, |
| | (Th17) | IL 17RB | endothelium, | ↑ inflammatory cytokines |
| IL-18 | Macrophages | CDw218a/IL18R1 | Th1 cells, | Induces production of IFNγ, |
| | | | NK cells | ↑ NK cell activity |
| IL-19 | — | IL20R | | — |
| IL-20 | Activated | IL20R | | regulates proliferation and differentiation of |
| | keratinocytes and | | | keratinocytes |
| | monocytes | | | |
| IL-21 | activated T helper | IL21R | All lymphocytes, | costimulates activation and proliferation of |
| | cells, | | dendritic cells | CD8+ T cells, augment NK cytotoxicity, |
| | NKT cells | | | augments CD40-driven B cell proliferation, |
| | | | | differentiation and isotype switching, promotes |
| | | | | differentiation of Th17 cells |
| IL-22 | T helper 17 cells | IL22R | | Production of defensins. Activates STAT1 and |
| | (Th17) | | | STAT3; increases production of acute phase |
| | | | | proteins, such as serum amyloid A, Alpha 1- |
| | | | | anti-chymotrypsin and haptoglobin |
| IL-23 | macrophages, | IL23R | | Maintenance of IL-17 producing cells, |
| | dendritic cells | | | increases angiogenesis but reduces CD8 T-cell |
| | | | | infiltration |
| IL-24 | melanocytes, | IL20R | | Plays important roles in tumor suppression, |
| | keratinocytes, | | | wound healing and psoriasis by influencing cell |
| | monocytes, | | | survival, inflammatory cytokine expression. |
| | T cells | | | |
| IL-25 | T Cells, | LY6E | | Induces the production IL-4, IL-5 and IL-13, |
| | mast cells, | | | which stimulate eosinophil expansion |
| | eosinophils, | | | |
| | macrophages, | | | |
| | mucosal epithelial | | | |
| | cells | | | |
| IL-26 | T cells, | IL20R1 | | Enhances secretion of IL-10 and IL-8 and cell |
| | monocytes | | | surface expression of CD54 on epithelial cells |
| IL-27 | macrophages, | IL27RA | | Regulates the activity of B lymphocyte and T |
| | dendritic cells | | | lymphocytes |
| IL-28 | — | IL28R | | Plays a role in immune defense against viruses |
| IL-29 | — | | | Plays a role in host defenses against microbes |
| IL-30 | — | | | Forms one chain of IL-27 |
| IL-31 | Th2 cells | IL31RA | | May play a role in inflammation of the skin |
| IL-32 | — | | | Induces monocytes and macrophages to |
| | | | | secrete TNF-α, IL-8 and CXCL2 |
| IL-33 | epithelial cells | | | Induces helper T cells to produce type 2 |
| | | | | cytokine |
| IL-35 | regulatory T cells | | | Suppression of T helper cell activation |
| IL-36 | — | | | Regulates DC and T cell responses |

In embodiments, once the cell is activated it will produce surface markers, cytoplasmic proteins, differences in mRNA and lor DNA expression profiles that indicate the cell has been activated. The surface markers can be analyzed by flow cytometry to measure the degree of activation with precision. Further, the degree of cytoplasmic cytotoxic protein production can also be analyzed to measure the degree of activation, using any of the following individually or in combination via flow cytometry, immunoblotting, mass spectroscopy, ELISA, etc. Further, the mRNA levels can be measured using standard mRNA detection and amplification procedures such as nested or qPCR (but not limited to these). Further, the differences in genetic profiling between resting and activated cells can be analyzed to determine the degree of activation. This can be accomplished with DNA expression arrays or similar technologies.

In embodiments, the same measurements as above (minus the genetic measurements, since BioNVs do not contain cellular nucleic acids) can be used to detect and monitor the degree of cellular activation can be applied to the BioNV as well. By measuring the same markers of activation, indirect measurement is achieved.

In embodiments, the therapeutic exosome encapsulates a payload; e.g., “lumen-loading”, or the ability of the exosome to have a payload loaded into the lumen (space in the biomimetic nanovesicle). In embodiments, the payload is one or more of a biologic, a nucleic acid, a fusion protein, a fluorescent protein, a tracing dye, a radionuclide, and/or a small molecule. In embodiments, the payload is a therapeutic payload for a disease type that the CAR is targeted against. In embodiments, the payload comprises one or more of an alkylating agent, an anthracycline, an antimetabolite, an anti-tumor antibiotic, an antibody or antibody format, a corticosteroid, a plant alkaloid, a topoisomerase inhibitor, a checkpoint inhibitor, an anti-infective agent, and/or a growth factor.

In embodiments, the nucleic acid payload encodes one or more of a CRISPR/Cas component, guide RNA (gRNA), tracer RNA (tracrRNA), micro RNA (miRNA), RNA inference (RNAi), small interference RNA (siRNA), duplex RNA, Piwi-interacting RNA (piRNA), small nuclear RNA (snRNA), small nucleolar RNA (snoRNA), antisense oligonucleotide (ASO), locked nucleic acid (LNA), splice switching oligonucleotide (SSO), tRNA, ribosomal RNA (rRNA), short hairpin (shRNA) complementary messenger RNA, repeat associated small interfering RNA (rasiRNA), and small non-coding RNA.

In embodiments, the one or more gene editors is a site-directed endonuclease, TALEN, ZFN, RNase P RNA, CRISPR/Cas nuclease, C2c1, C2c2, C2c3, Cas9, Cpf1, TevCas9, Archaea Cas9, CasY.1, CasY.2, CasY.3, CasY.4, CasY.5, CasY.6, CasX, Cas omega, transposase, and/or any ortholog or homolog thereof. In embodiments, the gene editors can also include gRNA, which, as used herein, refers to guide RNA. In embodiments, the gRNA can be a sequence complimentary to a coding or a non-coding sequence and can be tailored to the particular sequence to be targeted. In embodiments, the gRNA can be a sequence complimentary to a protein coding sequence, for example, a sequence encoding one or more viral structural proteins, (e.g., gag, pol, env and tat). In embodiments, the gRNA sequence can be a sense or anti-sense sequence. In embodiments, when a gene editor composition is administered herein, preferably without limitation, including two or more gRNAs; however, a single gRNA can also be used.

In embodiments, the gene editing payload comprises a transactivating response region (TAR) loop system. In embodiments, the therapeutic BioNV/exosome encapsulates a plasmid which expresses a gene editor and contains a TAR loop sequence between the 5′ end of the promoter and the gene editor/guide cassette and acts as a barrier, blocking transcription. In embodiments, transcription will only trigger in cells that are infected and contain the HIV Tat protein. In embodiments, the Tat protein binds to the TAR Loop, relaxes it, and frees the promoter for transcription, thereby expressing the editor and its guides.

In embodiments, BioNVs/exosomes can encapsulate disease-specific therapeutic payloads, for example, an increase in perforin or granzyme from an activated lymphocyte can be packaged into the resultant BioNVs/exosome to puncture cancer cells. In embodiments, an increase in cellular production of alarmins can be packaged into the resultant BioNV/exosome to enhance recruitment of additional lymphocytes to the site of a solid tumor. In embodiments, cells can be modified to increase production of gene editing payloads so that BioNVs/exosomes can incorporate gene editing material for tissue-specific targeting.

In embodiments, focused cellular regulation can be necessary if a single type of cytokine, or a small subset of cytokines, is desired that has therapeutically relevant value to be encapsulated within the lumen of a BioNV/exosome, instead of a broad/global expression method that mostly accompanies aa large series of regulation scenarios.

In embodiments, therapeutically relevant biomolecules (e.g., cytokines) can be selectively expressed from within any given cell by manipulating regulation points within points of signaling transduction. In embodiments, one point (or multiple points) within a single signaling pathway (or multiple pathways) can be activated, for example, the activation of a kinase, (de)activation of a phosphorylase, activation of a hydrolase (such as GTPase), introduction of an inhibitor (to diverge or block signals from one pathway to another), or an accelerating activator/agonist. In embodiments, these signaling pathways can be selectively activated by supplying cells with proteins, peptides, small molecules, nucleic acids, carbohydrates, chimeric molecules, viral ligands, inorganic elements/compounds (e.g., calcium), etc., either individually or in combination, to focus cellular pathways to drive expression of therapeutically relevant biomolecules within the cell which can then be packaged into the lumen of a BioNV/exosome (FIG. 3). In embodiments, the points of regulation can include, but are not limited to, one or more cell surface receptors targeted either individually, or in combination, to focus a signal within the cell to express the biomolecules of interest. In embodiments, the cells can be genetically modified so that they are especially sensitive to activation of these pathways resulting in a metabolically/phenotypically tuned cell (of any kind) which BioNVs/exosomes can be made from. In embodiments, cell can be made “especially sensitive” to activation by certain stimuli by overexpression of cell surface receptors, overexpression of intracellular signaling molecules (e.g., STATs, NF-κB, MAPK/ERK/ATM kinases, etc.), and/or constitutively active mutants thereof.

In embodiments, regulation points within a cell can be engineered to express specific therapeutically relevant proteins of interest as a stable cell line (e.g., stable integration for constitutive express).

In embodiments, transcription factors can be activated within a cell by supplying a small molecule, (de)phosphorylation event, or nucleic acid, etc. in embodiments, transcription factors can be transiently expressed by plasmids. In embodiments, transcription factors can be stably expressed from integration (e.g., with constitutively active promoters) to generate a stable cell line with constitutive signaling of signaling pathways related to therapeutic biomolecules.

In embodiments, the promoter region or enhancer regions of a gene can be regulated by the overexpression of a specific transcription factor, regulated by microRNAs, regulated by tRNAs, activated or inhibited using (g)RNA guided endonucleases with activating or inhibitory domains linked to them (e.g., CRIPSRa/CRISPRi).

In embodiments, a gene of therapeutically relevant interest can be integrated (either stably or transiently) into the cell of interest (based on desirable properties such as membrane proteins that infer barrier crossing). In embodiments, stably integrated genes can be activated by any one of method described herein.

In embodiments, background (unwanted) mRNAs can be silenced with interfering RNAs (e.g., siRNA, RNAi, etc.), to enhance the presence/expression of desired mRNAs to proteins of interest. In embodiments, mRNAs can also be regulated through IRES elements. In embodiments, specific spliced variants can be enhanced by the addition of IRES enhancing and/or repressing biomolecules, to produce a desired therapeutically relevant peptide/protein that can be packaged into a BioNV/exosome during post-activated cellular processing.

In embodiments, unwanted genes can be knocked-out to enhance the expression of desired therapeutically relevant genes. Those skilled in the art will appreciate the methods available to for transient and stable knock-out of undesired genes and/or entire signaling pathways during the processing for BioNVs/exosomes.

In embodiments, focused activation through CAR constructs can be performed. In embodiments, the intercellular domains of a CAR construct can be designed to include activating domains that are specifically focused on a single (or multiple) signaling pathways. In embodiments, these constructs can be activated through the extracellular binding domain of the CRA construct (e.g., supplying the cells a specific stimuli).

In embodiments, methods for regulation of genes described herein to enhance the presence of therapeutically relevant biomolecules within a desired cell (that is metabolically geared to produce the desired therapeutically relevant biomolecules) can be used to increase the concentration (or regulate to a desired concentration) of therapeutically relevant biomolecules, so that the regulated cell can then be processed into a BioNV or a naturally shed exosome that contains the desired concentrations of therapeutically relevant biomolecules within its lumen to treat mammalian diseases.

### Therapeutic BioNVs

In aspects, the present disclosure includes therapeutic BioNVs comprising (i) one or more membrane-embedded targeting agents targeted against one or more cellular biomarkers, (ii) one or more membrane-embedded proteins of an α-phagocytic integrin, CCL2, H2-M3, FasL, MFEG8, PD-L1 (in BioNVs derived from non-activated cell sources) and/or CTLA-4, SerpinB9, and anti-IL-6R antibody or antibody format, (iii) membrane-embedded protein of one of either CD24, CD47, CD200, chimeric CD24/CD47, chimeric CD24/CD200, and chimeric CD47/CD200, or two of either CD24, CD47, and CD200, and (iv) a membrane substantially lacking proteins of one or more of HLA-A, HLA-B, HLA-C, HLA-F, CIITA, IL-6, TRAC, TRBC, one of either HLA-E or HLA-G, and one or more of IL-4, IL-10, and IL-16, wherein the one or more therapeutically relevant biomolecules is a chemokine, interferon, interleukin, alarmin, lymphokine, perforin, tumor necrosis factor (TNF), colony-stimulating factor, bone morphogenic protein (BMP), erythropoietin (EPO), granulocyte-stimulating factor (G-CSF), granulocyte macrophage colony-stimulating factor (GM-CSF), gene editing payload, fusion protein, antibody or antibody format, or a combination thereof.

In aspects, the present disclosure includes therapeutic BioNVs comprising (i) one or more membrane-embedded targeting agents targeted against one or more cellular biomarkers, (ii) one or more membrane-embedded proteins of an α-phagocytic integrin, CCL2, H2-M3, FasL, MFEG8, PD-L1 (in BioNVs derived from non-activated cell sources) and/or CTLA-4, SerpinB9, and anti-IL-6R antibody or antibody format, (iii) a membrane-embedded protein of either CD24 and CD47, or chimeric CD24/CD47, and (iv) a membrane substantially lacking proteins of one or more of HLA-A, HLA-B, HLA-C, HLA-F, CIITA, IL-6, TRAC, TRBC, one of either HLA-E or HLA-G, SerpinB9 and CD200, and one or more of IL-4, IL-10, and IL-16, wherein the one or more therapeutically relevant biomolecules is a chemokine, interferon, interleukin, alarmin, lymphokine, perforin, granzyme, granulysin, tumor necrosis factor (TNF), colony-stimulating factor, bone morphogenic protein (BMP), erythropoietin (EPO), granulocyte-stimulating factor (G-CSF), granulocyte macrophage colony-stimulating factor (GM-CSF), gene editing payload, fusion protein, antibody or antibody format, or a combination thereof.

In embodiments, BioNVs that express PD-L1 are derived from non-activated cells and the BioNVs substantially lack perforin and/or granzyme.

In embodiments, the BioNV expresses and/or has activity of one or more antigen, ligand, and/or receptor of a tumor cell or cancer tissue. In embodiments, the one or more antigen, ligand, and/or receptor of a tumor cell or cancer tissue is listed in Table 4, Table 5, and/or Table 6. In embodiments, the one or more antigen, ligand, and/or receptor of a tumor cell or cancer tissue is a neoantigen, such as a mutant variant or peptide specific to a cancer or disease state that is otherwise not present in a subject to be treated. In embodiments, the BioNV expresses and/or has activity of a co-stimulatory molecule for immune cells. In embodiments, the co-stimulatory molecule for immune cells is IL-15.

In embodiments, BioNVs contain one or more outwardly facing, membrane-embedded targeting agents (e.g., CARs) capable of binding one or more target molecules. In embodiments, BioNVs contain outwardly facing, membrane-embedded CARs capable of binding a target molecule. In embodiments, therapeutic BioNVs are biomimetic due to the nanovesicle composition which originates from the plasma membrane of allogeneic, hypoimmunogenic modified cells. In embodiments, therapeutic BioNVs comprise plasma membrane-derived lipid bilayers, fully encapsulating an aqueous core which can house a variety of cell-derived molecules, including perforins, granzymes, cytokines, gene editing payloads, etc. In embodiments, the aqueous core of the therapeutic BioNVs can further enclose exogenous biologics, fluorescent proteins, tracing dyes, radionuclides, and small molecules, among other therapeutic agents.

In embodiments, the CAR constructs can comprise a variety of structural molecules. The structure-function of a prototypical CAR includes a fusion protein comprising an extracellular (or outwardly facing) binding moiety (e.g., scFv), connected by a hinge peptide (e.g., CH2/CH3 domains from an IgG Fc region, Gly-Gly-Ser peptide linkage, CD28 peptide, CD8a peptide, etc.) to a transmembrane domain (e.g., CD28, CD3ζ, CD4, CD8a, ICOS, etc.), followed by a variety of intracellular signaling domains (e.g., 4-1BB, CD3ζ, CD28, 4-1BB, ICOS, CD27, OX40, etc.). In embodiments, BioNVs lack the intracellular machinery of whole cells and therefore the CAR design does not necessitate any intracellular signaling molecules (primary CAR construct). In embodiments, the CAR construct includes an extracellular scFV binding moiety fused with an IgG CH2/CH3 linker to a CD28 transmembrane domain and substantially lacks any intracellular domains or functionality. In embodiments, the CAR constructs have the prototypical intracellular domains swapped or otherwise fused to anchor proteins, e.g., PLA2 domain from an AAV, fusion proteins, radionuclide-binding domains, cytoskeletal elements, small molecule transporting domains, etc., which may aid in the fusion to target cells and/or packaging and release of therapeutic payloads.

In embodiments, CAR antigen-binding molecules comprise a variety of binding moieties, including antibody-based or antibody format binding domains. In embodiments, therapeutic BioNVs comprise antibody or antibody format binding moieties selected from one or more of a monoclonal antibody, polyclonal antibody, antibody fragment, Fab, Fab′, Fab′-SH, F(ab′)2, Fv, single chain Fv (scFv), diabody, nanobody, linear antibody, bispecific antibody, multi-specific antibody, chimeric antibody, humanized antibody, human antibody, and fusion protein comprising the antigen-binding portion of an antibody. In embodiments, the CAR construct includes binding moieties with a Bispecific T cell Engager (BiTE), viral epitope recognition receptor (VERR), variable heavy chain IgG fragment VHH, VNAR, or through an engineered T-Cell Receptor (TCR).

To ensure proper directionality of CARs and to eliminate BioNVs lacking CARs, in embodiments, HPLC-based affinity chromatography techniques can be used to select and concentrate only the BioNVs with a sufficient surface concentration of solvent-exposed CARs. HPLC-based affinity chromatography techniques can be used to reduce the concentration of contaminating cell material and NVs which harbor immunogenic cell surface markers, either by positive or negative selection.

In embodiments, therapeutic BioNVs can include NVs with an outer plasma membrane leaflet only, an inner plasma membrane leaflet only, and/or both leaflets of a plasma membrane lipid bilayer intact. iPSC-derived NVs, in embodiments, include additional lipid additives (e.g., phosphatidylethanolamine, phosphatidylcholine, phosphatidylinositols, ceramides, lecithin, etc.), non-ionic surfactants (e.g., sorbitan monostearate, octadecylamine, etc.), sterols (e.g., cholesterol, bile salt derivatives, etc.), polyols (e.g., maltodextrin, sorbitol, sucrose, mannitol, etc.) and proteins (e.g., serum albumin, etc.) added for improved physicochemical properties, such as thermal stability and therapeutic payload packaging/release. The amount of cholesterol and the length and saturation of the hydrocarbon chains of the phospholipids can affect the rigidity and the stability of the bilayer, and in turn the capability of the NVs to host and release drugs, biomolecules, and other therapeutic payloads. In embodiments, therapeutic BioNVs also incorporate zwitterionic lipids and methods of using zwitterionic lipids, for example, as described in US Patent Publication No. US 20130216607, the contents of which are herein incorporated by reference in its entirety. Correspondingly, functionalization of the hydrophilic heads of the lipids with polymers or biomolecules can provide additional features to the vesicle surface, thus shaping their interaction with blood components, tissues, and the immune system in vivo.

In embodiments, the therapeutic BioNV encapsulates a payload; e.g., “lumen-loading”, or the ability of the BioNV to have a payload loaded into the lumen (space in the biomimetic nanovesicle). In embodiments, the payload is one or more of a biologic, a nucleic acid, a fusion protein, a fluorescent protein, a tracing dye, a radionuclide, and/or a small molecule. In embodiments, the payload is a therapeutic payload for a disease type that the CAR is targeted against. In embodiments, the payload comprises one or more of an alkylating agent, an anthracycline, an antimetabolite, an anti-tumor antibiotic, an antibody or antibody format, a corticosteroid, a plant alkaloid, a topoisomerase inhibitor, a checkpoint inhibitor, an anti-infective agent, and/or a growth factor.

In embodiments, the nucleic acid payload encodes one or more of a CRISPR/Cas component, guide RNA (gRNA), tracer RNA (tracrRNA), micro RNA (miRNA), RNA inference (RNAi), small interference RNA (siRNA), duplex RNA, Piwi-interacting RNA (piRNA), small nuclear RNA (snRNA), small nucleolar RNA (snoRNA), antisense oligonucleotide (ASO), locked nucleic acid (LNA), splice switching oligonucleotide (SSO), tRNA, ribosomal RNA (rRNA), short hairpin (shRNA) complementary messenger RNA, repeat associated small interfering RNA (rasiRNA), and small non-coding RNA.

In embodiments, the payload includes gene editing nucleic acids and/or proteins, such as for example, TALENs, ZFNs, RNase P RNA, C2c1, C2c2, C2c3, Cas9, Cpf1, TevCas9, Archaea Cas9, CasY.1, CasY.2, CasY.3, CasY.4, CasY.5, CasY.6, CasX Cas omega, transposase, and/or any ortholog or homolog of any of these editors. In embodiments, the gene editors can also include gRNA, which, as used herein, refers to guide RNA. In embodiments, the gRNA can be a sequence complimentary to a coding or a non-coding sequence and can be tailored to the particular sequence to be targeted. In embodiments, the gRNA can be a sequence complimentary to a protein coding sequence, for example, a sequence encoding one or more viral structural proteins, (e.g., gag, pol, env and tat). In embodiments, the gRNA sequence can be a sense or anti-sense sequence. In embodiments, when a gene editor composition is administered herein, preferably without limitation, including two or more gRNAs; however, a single gRNA can also be used.

In embodiments, the therapeutic BioNV comprises one or more perforin molecules and/or one or more granzyme molecules derived from a cell from which the BioNV is derived. In embodiments, the therapeutic BioNV comprises one or more perforin molecules and one and/or more granzyme molecules exogenously added to the BioNV. In embodiments, BioNVs designed to carry granzymes lack CD200 and SerpinB9.

In embodiments, the therapeutic BioNV can deliver a gene editing payload comprising a transactivating response region (TAR) loop system. In embodiments, the therapeutic BioNV encapsulates a plasmid which expresses a gene editor and contains a TAR loop sequence between the 5′ end of the promoter and the gene editor/guide cassette and acts as a barrier, blocking transcription. In embodiments, transcription will only trigger in cells that are infected and contain the HIV Tat protein. In embodiments, the Tat protein binds to the TAR Loop, relaxes it, and frees the promoter for transcription, thereby expressing the editor and its guides.

In embodiments, primary targeted therapeutic BioNVs can be used to deliver small molecule therapeutic payloads. In embodiments, second generation (or 3rd or 4th gen) CAR-containing therapeutic BioNVs derived from activated lymphocytes can contain cytokines and other cytotoxic peptides. In embodiments, therapeutic BioNVs can be formatted to encapsulate and deliver plasmid DNA, for example, to express gene editing nucleases and gRNA in target cells. Alternatively, or additionally, in embodiments, therapeutic BioNVs can encapsulate the nucleases and gRNA. In embodiments, targeted second generation (or 3rd or 4th gen) therapeutic BioNVs can be designed to encapsulate and deliver additional therapeutic proteins or peptides of interest.

In embodiments, the therapeutic BioNV substantially lacks the above to be allogeneic and/or hypoimmune. In embodiments, the therapeutic BioNV does not produce an adverse immune reaction upon infusion into a subject for treating a disease.

In embodiments, the therapeutic BioNV is formed by processing the activated hypoimmunogenic cell by one or more of sonication, adaptive focused acoustics technology, French press, extrusion, serial extrusion, enzymatic rupture of cells (e.g., trypsinization), cell lysis by detergent, and/or electroporation. In embodiments, the cell disruption is by serial extrusion, for example, if the BioNVs are derived from whole cells using the serial extrusion process, the diminishing pore size of the polycarbonate filters coincide with the diameter and volume of the BioNV.

In embodiments, therapeutic BioNVs can be analyzed for homogeneity of size by dynamic light scattering (DLS), flow cytometry, mass photometry, among other methods of determining particle size. In embodiments, therapeutic BioNVs can be filtered for a particle size, or range of sizes, to optimize renal clearance and other clinically-relevant NV properties. In embodiments, therapeutic BioNVs are about 20 nm to 1200 nm in size. In embodiments, therapeutic BioNVs are about 10 nm in size, about 20 nm in size, about 30 nm in size, about 40 nm in size, about 50 nm in size, about 60 nm in size, about 70 nm in size, about 80 nm in size, about 90 nm in size, about 100 nm in size, about 120 nm in size, about 140 nm in size, about 160 nm in size, about 180 nm in size, about 200 nm in size, about 300 nm in size, about 400 nm in size, about 500 nm in size, about 600 nm in size, about 700 nm in size, about 800 nm in size, about 900 nm in size, about 1000 nm in size, about 1100 nm in size, or about 1200 nm in size. In embodiments, therapeutic BioNVs range in size from about 10 nm to 20 nm in size, about 20 nm to 30 nm in size, about 30 nm to 40 nm in size, about 40 nm to 50 nm in size, about 50 nm to 60 nm in size, about 60 nm to 70 nm in size, about 70 nm to 80 nm in size, about 80 nm to 90 nm in size, about 90 nm to 100 nm in size, about 10 nm to 100 nm in size, about 100 nm to 200 nm in size, about 200 nm to 400 nm in size, about 400 nm to 600 nm in size, about 600 nm to 800 nm in size, about 800 nm to 1000 nm in size, or about 1000 nm to 1200 nm in size.

### Therapeutic Exosomes

In aspects, the present disclosure includes therapeutic exosomes comprising (i) one or more membrane-embedded targeting agents targeted against one or more cellular biomarkers, (ii) one or more membrane-embedded proteins of an α-phagocytic integrin, CCL2, H2-M3, FasL, MFEG8, PD-L1 (in BioNVs derived from non-activated cell sources) and/or CTLA-4, SerpinB9, and anti-IL-6R antibody or antibody format, (iii) a membrane-embedded protein of one of either CD24, CD47, CD200, chimeric CD24/CD47, chimeric CD24/CD200, and chimeric CD47/CD200, or two of either CD24, CD47, and CD200, (iv) a membrane substantially lacking proteins of one or more of HLA-A, HLA-B, HLA-C, HLA-F, CIITA, IL-6, TRAC, TRBC, one of either HLA-E or HLA-G, and one or more of IL-4, IL-10, and IL-16, wherein the one or more therapeutically relevant biomolecules is a chemokine, interferon, interleukin, alarmin, lymphokine, perforin, tumor necrosis factor (TNF), colony-stimulating factor, bone morphogenic protein (BMP), erythropoietin (EPO), granulocyte-stimulating factor (G-CSF), granulocyte macrophage colony-stimulating factor (GM-CSF), gene editing payload, fusion protein, antibody or antibody format, or a combination thereof.

In aspects, the present disclosure includes therapeutic exosomes comprising (i) one or more membrane-embedded targeting agents targeted against one or more cellular biomarkers, (ii) one or more membrane-embedded proteins of an α-phagocytic integrin, CCL2, H2-M3, FasL, MFEG8, PD-L1 (in BioNVs derived from non-activated cell sources) and/or CTLA-4, SerpinB9, and anti-IL-6R antibody or antibody format, (iii) a membrane-embedded protein of either CD24 and CD47, or chimeric CD24/CD47, and (iv) a membrane substantially lacking proteins of one or more of HLA-A, HLA-B, HLA-C, HLA-F, CIITA, IL-6, TRAC, TRBC, one of either HLA-E or HLA-G, SerpinB9 and CD200, and one or more of IL-4, IL-10, and IL-16, wherein the one or more therapeutically relevant biomolecules is a chemokine, interferon, interleukin, alarmin, lymphokine, perforin, granzyme, granulysin, tumor necrosis factor (TNF), colony-stimulating factor, bone morphogenic protein (BMP), erythropoietin (EPO), granulocyte-stimulating factor (G-CSF), granulocyte macrophage colony-stimulating factor (GM-CSF), gene editing payload, fusion protein, antibody or antibody format, or a combination thereof.

In embodiments, exosomes that express PD-L1 are derived from non-activated cells and the exosomes substantially lack perforin and/or granzyme.

In embodiments, the exosome expresses and/or has activity of one or more antigen, ligand, and/or receptor of a tumor cell or cancer tissue. In embodiments, the one or more antigen, ligand, and/or receptor of a tumor cell or cancer tissue is listed in Table 4, Table 5, and/or Table 6. In embodiments, the one or more antigen, ligand, and/or receptor of a tumor cell or cancer tissue is a neoantigen, such as a mutant variant or peptide specific to a cancer or disease state that is otherwise not present in a subject to be treated. In embodiments, the exosome expresses and/or has activity of a co-stimulatory molecule for immune cells. In embodiments, the co-stimulatory molecule for immune cells is IL-15. Exosomes that express and/or have activity of one or more neoantigens and-or co-stimulatory molecules, in embodiments, can be used in the production of whole cells, as described herein, and/or BioNVs, e.g., by neoantigen-targeted activation/stimulation.

In embodiments, exosomes contain one or more outwardly facing, membrane-embedded targeting agents (e.g., CARs) capable of binding one or more target molecules. In embodiments, exosomes contain outwardly facing, membrane-embedded CARs capable of binding a target molecule. In embodiments, therapeutic exosomes are biomimetic due to the nanovesicle composition which originates from the plasma membrane of allogeneic, hypoimmunogenic modified cells. In embodiments, therapeutic exosomes comprise plasma membrane-derived lipid bilayers, fully encapsulating an aqueous core which can house a variety of cell-derived molecules, including perforins, granzymes, cytokines, gene editing payloads, etc. In embodiments, the aqueous core of the therapeutic exosomes can further enclose exogenous biologics, fluorescent proteins, tracing dyes, radionuclides, and small molecules, among other therapeutic agents.

In embodiments, CAR constructs can comprise a variety of structural molecules. The structure-function of a prototypical CAR includes a fusion protein comprising an extracellular (or outwardly facing) binding moiety (e.g., scFv), connected by a hinge peptide (e.g., CH2/CH3 domains from an IgG Fc region, Gly-Gly-Ser peptide linkage, CD28 peptide, CD8a peptide, etc.) to a transmembrane domain (e.g., CD28, CD3ζ, CD4, CD8a, ICOS, etc.), followed by a variety of intracellular signaling domains (e.g., 4-1BB, CD3ζ, CD28, 4-1BB, ICOS, CD27, OX40, etc.). In embodiments, therapeutic exosomes lack the intracellular machinery of whole cells and therefore the CAR design does not necessitate any intracellular signaling molecules (primary CAR construct). In embodiments, the CAR construct includes an extracellular scFV binding moiety fused with an IgG CH2/CH3 linker to a CD28 transmembrane domain and substantially lacks any intracellular domains or functionality. In embodiments, the CAR constructs have the prototypical intracellular domains swapped or otherwise fused to anchor proteins, e.g., PLA2 domain from an AAV, fusion proteins, radionuclide-binding domains, cytoskeletal elements, small molecule transporting domains, etc., which may aid in the fusion to target cells and/or packaging and release of therapeutic payloads.

In embodiments, CAR antigen-binding molecules comprise a variety of binding moieties, including antibody-based or antibody format binding domains. In embodiments, therapeutic exosomes comprise antibody or antibody format binding moieties selected from one or more of a monoclonal antibody, polyclonal antibody, antibody fragment, Fab, Fab′, Fab′-SH, F(ab′)2, Fv, single chain Fv (scFv), diabody, nanobody, linear antibody, bispecific antibody, multi-specific antibody, chimeric antibody, humanized antibody, human antibody, and fusion protein comprising the antigen-binding portion of an antibody. In embodiments, the CAR construct includes binding moieties with a Bispecific T cell Engager (BiTE), viral epitope recognition receptor (VERR), variable heavy chain IgG fragment VHH, VNAR, or through an engineered T-Cell Receptor (TCR).

To ensure proper directionality of CARs and to eliminate exosomes lacking CARs, in embodiments, HPLC-based affinity chromatography techniques can be used to select and concentrate only the exosomes with a sufficient surface concentration of solvent-exposed CARs. HPLC-based affinity chromatography techniques can be used to reduce the concentration of contaminating cell material and NVs which harbor immunogenic cell surface markers, either by positive or negative selection.

In embodiments, therapeutic exosomes differ from therapeutic BioNVs due to being naturally shed from cells via canonical vesicular pathways, i.e., exosomes can be harvested from cells without irreversibly mechanically disrupting (e.g., rupturing, shearing, etc.) the plasma membrane of the cells from which they are derived. In embodiments, exosomes can be harvested from the culture media of the cells, for example, by differential ultracentrifugation of culture media.

In embodiments, harvesting the therapeutic exosomes from the activated, hypoimmunogenic cell comprises inducing hypoxia. In embodiments, therapeutic exosome formation can be increased from cells using numerous strategies such as inducing cellular stress.

In embodiments, harvesting the therapeutic exosomes from the activated, hypoimmunogenic cell comprises expressing or increasing expression of one or more cellular factors. In embodiments, overexpressing cellular factors such as tetraspanin CD9 or hypoxia-inducible factor-1a can induce exosome formation for harvesting.

In embodiments, harvesting the therapeutic exosomes from the activated, hypoimmunogenic cell comprises supplying one or more small molecule exosome modulators. In embodiments, supplying small molecule modulators can include fenoterol, norepinephrine, N-methyldopamine, and/or mephenesin.

In embodiments, harvesting the therapeutic exosomes from the activated, hypoimmunogenic cell comprises supplementing the media with one or more exosome factors. In embodiments, exosome factors can include nutritional supplementation to media, such as with forskolin. In embodiments, exosome factors can include providing specific cell types stimuli for increased production of therapeutic exosomes, for example, cardiac fibroblasts can be stimulated with TGF-β to increase collagen expression, which leads to exosome formation.

In embodiments, the therapeutic exosome encapsulates a payload; e.g., “lumen-loading”, or the ability of the exosome to have a payload loaded into the lumen (space in the biomimetic nanovesicle). In embodiments, the payload is one or more of a biologic, a nucleic acid, a fusion protein, a fluorescent protein, a tracing dye, a radionuclide, and/or a small molecule. In embodiments, the payload is a therapeutic payload for a disease type that the CAR is targeted against. In embodiments, the payload comprises one or more of an alkylating agent, an anthracycline, an antimetabolite, an anti-tumor antibiotic, an antibody or antibody format, a corticosteroid, a plant alkaloid, a topoisomerase inhibitor, a checkpoint inhibitor, an anti-infective agent, and/or a growth factor.

In embodiments, the nucleic acid payload encodes one or more of a CRISPR/Cas component, guide RNA (gRNA), tracer RNA (tracrRNA), micro RNA (miRNA), RNA inference (RNAi), small interference RNA (siRNA), duplex RNA, Piwi-interacting RNA (piRNA), small nuclear RNA (snRNA), small nucleolar RNA (snoRNA), antisense oligonucleotide (ASO), locked nucleic acid (LNA), splice switching oligonucleotide (SSO), tRNA, ribosomal RNA (rRNA), short hairpin (shRNA) complementary messenger RNA, repeat associated small interfering RNA (rasiRNA), and small non-coding RNA.

In embodiments, the payload includes gene editing nucleic acids and/or proteins, such as for example, TALENs, ZFNs, RNase P RNA, C2c1, C2c2, C2c3, Cas9, Cpf1, TevCas9, Archaea Cas9, CasY.1, CasY.2, CasY.3, CasY.4, CasY.5, CasY.6, CasX Cas omega, transposase, and/or any ortholog or homolog of any of these editors. In embodiments, the gene editors can also include gRNA, which, as used herein, refers to guide RNA. In embodiments, the gRNA can be a sequence complimentary to a coding or a non-coding sequence and can be tailored to the particular sequence to be targeted. In embodiments, the gRNA can be a sequence complimentary to a protein coding sequence, for example, a sequence encoding one or more viral structural proteins, (e.g., gag, pol, env and tat). In embodiments, the gRNA sequence can be a sense or anti-sense sequence. In embodiments, when a gene editor composition is administered herein, preferably without limitation, including two or more gRNAs; however, a single gRNA can also be used.

In embodiments, the therapeutic exosome can deliver a gene editing payload comprising a transactivating response region (TAR) loop system. In embodiments, the therapeutic BioNV encapsulates a plasmid which expresses a gene editor and contains a TAR loop sequence between the 5′ end of the promoter and the gene editor/guide cassette and acts as a barrier, blocking transcription. In embodiments, transcription will only trigger in cells that are infected and contain the HIV Tat protein. In embodiments, the Tat protein binds to the TAR Loop, relaxes it, and frees the promoter for transcription, thereby expressing the editor and its guides.

In embodiments, primary targeted therapeutic exosomes can be used to deliver small molecule therapeutic payloads. In embodiments, second generation (or 3rd or 4th gen) CAR-containing therapeutic exosomes derived from activated lymphocytes can contain cytokines and other cytotoxic peptides. In embodiments, therapeutic exosomes can be formatted to encapsulate and deliver plasmid DNA, for example, to express gene editing nucleases and gRNA in target cells. Alternatively, or additionally, in embodiments, therapeutic exosomes can encapsulate the nucleases and gRNA. In embodiments, targeted second generation (or 3rd or 4th gen) therapeutic exosomes can be designed to encapsulate and deliver additional therapeutic proteins or peptides of interest.

In embodiments, therapeutic exosome can be analyzed for homogeneity of size by dynamic light scattering (DLS), flow cytometry, mass photometry, among other methods of determining particle size. In embodiments, therapeutic exosomes can be filtered for a particle size, or range of sizes, to optimize renal clearance and other clinically-relevant NV properties. In embodiments, therapeutic exosomes are about 10 nm to 200 nm in size. In embodiments, therapeutic exosomes can be about 10 nm in size, about 20 nm in size, about 30 nm in size, about 40 nm in size, about 50 nm in size, about 60 nm in size, about 70 nm in size, about 80 nm in size, about 90 nm in size, about 100 nm in size, about 120 nm in size, about 140 nm in size, about 160 nm in size, about 180 nm in size, about 200 nm in size. In embodiments, therapeutic exosome range in size from about 10 nm to 20 nm in size, about 20 nm to 30 nm in size, about 30 nm to 40 nm in size, about 40 nm to 50 nm in size, about 50 nm to 60 nm in size, about 60 nm to 70 nm in size, about 70 nm to 80 nm in size, about 80 nm to 90 nm in size, about 90 nm to 100 nm in size, about 10 nm to 100 nm in size, about 100 nm to 200 nm in size.

### Methods of Treating Disease

In embodiments, therapeutic BioNVs/exosomes can be used to treat and/or prevent a disease or disorder. In embodiments, the disease or disorder is a cancer, infectious disease, hereditary disorder, or an orphan disease.

In embodiments, the cancer is one or more of a carcinoma, a sarcoma, a myeloma, a leukemia, a lymphoma, a mixed type cancer, and/or a metastatic cancer.

In embodiments, the cancer is selected from Acute Biphenotypic Leukemia, Acute Eosinophilic Leukemia, Acute Lymphoblastic Leukemia, Acute Lymphocytic Leukemia, Acute Myeloid Dendritic Cell Leukemia, Acute Myeloid Leukemia, Adenocarcinoma of the Lung, Adrenal Gland Tumors, Adrenocortical Carcinoma, AIDS-related Cancers, AIDS-related Lymphoma, Alveolar Soft Part and Cardiac Sarcoma, Amyloidosis, Anal Cancer, Anaplastic Large Cell Lymphoma, Angioimmunoblastic T-cell Lymphoma, Appendix Cancer, Astrocytoma, Ataxia-telangiectasia, Attenuated Familial Adenomatous Polyposis, B-cell Prolymphocytic Leukemia, Basal Cell Carcinoma, Beckwith-Wiedemann Syndrome, Bile Duct Cancer, Birt-Hogg-Dubb Syndrome, Bladder Cancer, Bone Cancer, Brain and Nervous System Cancer, Brain Stem Glioma, Brainstem Glioma, Brain Tumors, Breast Cancer, Bronchial Adenomas/Carcinoids, Burkitt's Lymphoma, Carcinoid Tumor, Carcinoid Tumors, Carney Complex, Central Nervous System Tumors, Cerebellar Astrocytoma, Cerebral Astrocytoma, Cervical Cancer, Childhood Desmoplastic Ganglioglioma, Cholangiocarcinoma, Chondrosarcoma, Chronic Lymphocytic Leukemia, Chronic Myelogenous Leukemia, Chronic Myeloid Leukemia, Chronic T-cell Lymphocytic Leukemia, Colon Cancer, Colorectal Cancer, Cowden Syndrome, Craniopharyngioma, Cutaneous T-cell Lymphoma, Dermatofibrosarcoma Protuberans, Desmoplastic Small Round Cell Tumor, Diffuse Gastric Cancer, Diffuse Large B-cell Lymphoma, Endocrine System Cancer, Endocrine Tumors, Endometrial Cancer, Eosinophilic Leukemia, Ependymoma, Epithelioid Hemangioendothelioma (EHE), Esophageal Cancer, Ewing Sarcoma, Extragonadal Germ Cell Tumor, Extrahepatic Bile Duct Cancer, Eye Cancer, Eyelid Cancer, Fallopian Tube Cancer, Familial Adenomatous Polyposis, Familial Malignant Melanoma, Familial Clear Cell Renal Cell Carcinoma (RCC), Follicular Lymphoma, Gallbladder Cancer, Gardner Syndrome, Gastric (stomach) Cancer, Gastrointestinal Cancer, Gastrointestinal Carcinoid Tumor, Gastrointestinal Stromal Tumor, Gastrointestinal Stromal Tumor (GIST), Genitourinary and Gynecologic Cancer, Germ Cell Tumor, Gestational Trophoblastic Disease, Gestational Trophoblastic Tumor, Glioblastoma, Glioma, Hairy Cell Leukemia, Head and Neck Cancer, Hematopoietic Cancer, Hepatocellular Cancer, Hepatosplenic T-cell Lymphoma, HIV-related Cancer, Hodgkin's Lymphoma, Hypopharyngeal Cancer, Inflammatory Breast Cancer, Intravascular Large B-cell Lymphoma, Invasive Cribriform Carcinoma, Invasive Lobular Carcinoma, Islet Cell Carcinoma (endocrine Pancreas), Islet Cell Tumors, Juvenile Polyposis Syndrome, Kaposi Sarcoma, Keratoacanthoma, Kidney Cancer, Lacrimal Gland Tumor, Large Granular Lymphocytic Leukemia, Laryngeal and Hypopharyngeal Cancer, Leiomyomatosis and Renal Cell Cancer, Leiomyosarcoma, Li-Fraumeni Syndrome (LFS), Liposarcoma, Liver Cancer, Lung Cancer, Lymphomas of Primary Cutaneous Origin, Lymphomatoid Granulomatosis, Lymphoplasmacytic Lymphoma, Lynch Syndrome, Malignant Fibrous Histiocytoma of Bone, Mantle Cell Lymphoma, Marginal Zone B-cell Lymphoma, Mast Cell Leukemia, Mastocytosis, Mediastinal Large B Cell Lymphoma, Medullary Carcinoma, Medulloblastoma, Melanoma, Meningioma, Merkel Cell Carcinoma, Mesothelioma, Mixed Polyposis Syndrome, Mucosa-associated Lymphoid Tissue Lymphoma, Muir-Torre Syndrome (MTS), Multiple Endocrine Neoplasia Syndrome, Multiple Endocrine Neoplasia Type 1, Multiple Endocrine Neoplasia Type 2, Multiple Myeloma, Multiple Myeloma/Plasma Cell Neoplasm, Mycosis Fungoides, Myelodysplastic Syndromes, MYH-associated Polyposis, Myxosarcoma, Nasal Cavity and Paranasal Sinus Cancer, Nephroblastoma, Nasopharyngeal Cancer, Nasopharyngeal Carcinoma, Neuroblastoma, Neuroendocrine Tumors, Neurofibromatosis Type 1, Neurofibromatosis Type 2, Nevoid Basal Cell Carcinoma Syndrome, Nodal Marginal Zone B Cell Lymphoma, Non-Hodgkin Lymphoma, Non-small Cell Lung Cancer, Non-small Cell Lung Carcinoma, Oligodendroglioma, Optic Nerve Glioma, Oral and/or Oropharyngeal Cancer, Osteosarcoma, Ovarian Cancer, Ovarian Epithelial Cancer, Ovarian Germ Cell Tumor, Pancreatic Cancer, Papillary Renal Cell Carcinoma, Paranasal Sinus and Nasal Cavity Cancer, Parathyroid Cancer, Pelvic Cancer, Penile Cancer, Peutz-Jeghers Syndrome, Pharyngeal Cancer, Pheochromocytoma, Phyllodes Tumor, Pilocytic Astrocytoma, Pineal Astrocytoma, Pituitary Adenoma, Pituitary Gland Tumors, Plasmablastic Lymphoma, Pleuropulmonary Blastoma, Precursor B Lymphoblastic Leukemia, Primary Central Nervous System Lymphoma, Primary Cutaneous Follicular Lymphoma, Primary Cutaneous Immunocytoma, Primary Effusion Lymphoma, Primitive Neuroectodermal Tumor, Prostate Cancer, Rectal Cancer, Renal Cancer, Renal Cell Carcinoma, Retinoblastoma, Rhabdomyosarcoma, Salivary Gland Cancer, Sarcoma, Sarcomas of Primary Cutaneous Origin, Sebaceous Carcinoma, Sézary Syndrome, Skin Adnexal Tumors, Skin Cancer, Small Bowel Cancer, Small Cell Lung Cancer, Small Intestine Cancer, Splenic Marginal Zone Lymphoma, Squamous-cell Carcinoma of the Lung, Squamous Cell Carcinoma, Squamous Cell Skin Cancer, Stomach Cancer, Surface Epithelial-Stromal Tumor, T-cell Prolymphocytic Leukemia, Testicular Cancer, Thoracic and Respiratory Cancer, Thymoma, Thymic Carcinoma, Thyroid Cancer, Transitional Cell Cancer, Transitional Cell Canceradenoid Cystic Carcinoma, Tuberous Sclerosis Syndrome, Tubular Carcinoma, Turcot Syndrome, Unknown Primary Cancer, Unsorted Cancer, Ureter Cancer, Urethral Cancer, Uterine Cancer, Uterine Sarcoma, Uveal Melanoma, Vaginal Cancer, Visual Pathway and Hypothalamic Glioma, Von Hippel-Lindau (VHL) Syndrome, Vulvar Cancer, Wilms Tumor, and Xeroderma Pigmentosum.

In embodiments, the BioNVs can target cancer cells associated with adenoid cystic carcinoma, adrenal gland tumors, amyloidosis, anal cancer, appendix cancer, astrocytoma, ataxia-telangiectasia, attenuated familial adenomatous polyposis, Beckwith-Wiedemann Syndrome, bile duct cancer, Birt-Hogg-Dube Syndrome, bladder cancer, bone cancer, brain stem glioma, brain tumors, breast cancer, carcinoid tumors, Carney complex, central nervous system tumors, cervical cancer, colorectal cancer, Cowden syndrome, craniopharyngioma, desmoplastic infantile ganglioglioma, endocrine tumors, ependymoma, esophageal cancer, Ewing sarcoma, eye cancer, eyelid cancer, fallopian tube cancer, familial adenomatous polyposis, familial malignant melanoma, familial non-VHL clear cell renal cell carcinoma, gallbladder cancer, Gardner Syndrome, gastrointestinal stromal tumor, germ cell tumor, gestational trophoblastic disease, head and neck cancer, diffuse gastric cancer, leiomyomatosis and renal cell cancer, mixed polyposis syndrome, pancreatitis, papillary renal cell carcinoma, HIV and AIDS-related cancer, islet cell tumors, juvenile polyposis syndrome, kidney cancer, lacrimal gland tumor, laryngeal and hypopharyngeal cancer, acute lymphoblastic leukemia, acute lymphocytic leukemia, acute myeloid leukemia, B-cell prolymphocytic leukemia, hairy cell leukemia, chronic lymphocytic leukemia, chronic myeloid leukemia, chronic T-cell lymphocytic leukemia, eosinophilic leukemia, Li-Fraumeni Syndrome, liver cancer, lung cancer, Hodgkin lymphoma, Non-Hodgkin lymphoma, Lynch Syndrome, mastocytosis, medulloblastoma, melanoma, meningioma, mesothelioma, Muir-Torre Syndrome, multiple endocrine neoplasia type 1, multiple endocrine neoplasia type 2, multiple myeloma, myelodysplastic syndromes, MYH-associated polyposis, nasal cavity and paranasal sinus cancer, nasopharyngeal cancer, neuroblastoma, neuroendocrine tumors, neurofibromatosis type 1, neurofibromatosis type 2, nevoid basal cell carcinoma syndrome, oral and oropharyngeal cancer, osteosarcoma, ovarian cancer, pancreatic cancer, parathyroid cancer, penile cancer, Peutz-Jeghers Syndrome, pituitary gland tumors, pleuropulmonary blastoma, prostate cancer, retinoblastoma, rhabdomyosarcoma, salivary gland cancer, sarcoma, alveolar soft part and cardiac sarcoma, Kaposi sarcoma, skin cancer, small bowel cancer, stomach cancer, testicular cancer, thymoma, thyroid cancer, tuberous sclerosis syndrome, Turcot Syndrome, unknown primary, uterine cancer, vaginal cancer, Von Hippel-Lindau Syndrome, Wilms tumors, or Xeroderma pigmentosum.

In embodiments, therapeutic BioNVs/exosomes used to treat and/or prevent a disease or disorder are targeted against one or more biomarkers of Table 5 and/or Table 6.

TABLE 5
Illustrative checkpoint biomarkers and
ligands of checkpoint biomarkers.

| Biomarker | Illustrative Function |
|---|---|
| CTLA-4 | Immune checkpoint |
| PD-L1 | Suppress adaptive immune system |
| PD-L2 | Immune checkpoint receptor ligand |
| B7-H3 | Immune checkpoint |
| B7-H4 | Immune co-inhibitory molecule |
| BTLA | Immune regulatory receptor |
| HVEM | Receptor binding TNF receptor associated factor |
| | molecules |
| TIM3 | Immune response regulator |
| GAL9 | Induces T-cell death |
| LAG3 | Immune checkpoint |
| VISTA | Immune regulatory receptor |
| KIR | Regulate immune tolerance |
| 2B4 | Regulate NK cells |
| CD160 | Checkpoint inhibitor |
| CGEN-15049 | Immune checkpoint |
| CHK1 | Initiate DNA damage response |
| CHK2 | Regulates cell division |
| A2aR | Signals adenosine |
| B-7 family ligands | Regulate immune response |

TABLE 6
Illustrative cancer cell biomarkers.

| Biomarker | Illustrative Function |
|---|---|
| AFP (alpha- | Hepatocellular carcinoma (HCC) |
| feroprotein) | |
| Mesothelin | mesothelioma |
| ER (estrogen | Breast cancer |
| receptor) | |
| PR (progesterone | Breast cancer |
| receptor) | |
| HER-2/neu | Breast cancer |
| EGFR (epidermal | non-small-cell lung cancer |
| growth factor | |
| receptor) | |
| KRAS | non-small-cell lung cancer, colorectal cancer |
| UGT1A1 | colorectal cancer |
| c-KIT | Blood, gastric, skin cancers |
| CD30 | lymphomas |
| PDGFR (platelet | Gastrointestinal cancer |
| derived growth | |
| factor receptor) | |
| TEM8 (tumor | lung, breast, and colorectal cancer |
| endothelial | |
| marker 8) | |
| EIIIB | Various solid tumors |
| CA-125 | Ovarian cancer |
| CD2 | Adhesion between T cells and other cell types |
| | and T-cell activation |
| CD9 | Renal cell carcinoma |
| CD11 b | Various tumors |
| CD18 | Various tumors |
| CD19 | lymphomas |
| CD20 | lymphomas |
| CD21 | lymphomas |
| CD22 | endometrial cancer, intestinal cancer, skin |
| | cancer, leukemia |
| CD24 | Regulation of B-cell proliferation and |
| | differentiation |
| CD30 | lymphomas |
| CD31 | Angiosarcoma, Cell adhesion. Plays a key role in |
| | leukocyte trafficking across endothelium |
| CD32a | Dormant T cells harboring viruses |
| CD38 | Cell adhesion and signal transduction |
| CD41 | Ovarian cancer |
| CD42b | lymphomas |
| CD43 | Breast, colon, gastric, lung cancers |
| CD44 | Cell adhesion and migration |
| CD48 | Cell adhesion and T-cell costimulation |
| CD51 | Colorectal cancer |
| CD54 | Colorectal cancer, Cell adhesion, lymphocyte |
| | activation, and migration |
| CD61 | Breast, ovarian cancer |
| CD62P | Platelet activation |
| CD63 | Lung cancer |
| CD66a | Neutrophil activation, homophilic and heterophilic |
| | adhesion |
| CD66b | Cell adhesion and neutrophil activation |
| CD66c | Cell adhesion and neutrophil activation |
| CD66e | Homophilic and heterophilic adhesion. May play |
| | a role in the metastasis of cancer cells |
| CD74 | MHC class II antigen processing |
| CD87 | Cell chemotaxis and adhesion |
| CD96 | Involved in adhesive interactions of activated |
| | T cells and NK cells |
| CD98 | Cell activation and cell aggregation |
| CD105 | Angiogenesis. Regulatory component of TGF-beta |
| | receptor complex. Modulates cellular response |
| | to TGF beta 1 |
| CD106 | Leukocyte adhesion. Transmigration and |
| | costimulation of T cells. |
| CD107a | Degranulation marker |
| CD111 | Entry mediator for several herpes viruses (HSV1, |
| | HSV2, PRV, BHV1). Believed to be involved in cell |
| | adhesion mechanisms |
| CD112 | Intracellular adhesion, component of adherins |
| | junctions. Receptor for herpes simplex |
| | virus |
| CD117 | Crucial for development of gonadal and pigment |
| | stem cells |
| CD126 | Receptor for IL-6 |
| CD130 | Transmits activities of IL-6, IL-11, LIF and CNF |
| CD138 | Cell proliferation, cell migration, and cell-matrix |
| | interactions |
| CD140b | Cell proliferation, differentiation and survival |
| CD143 | Converts angiotensin I to angiotensin II by release of |
| | terminal His-Leu, which results in an increase of the |
| | vasoconstrictor activity of angiotensin. Cell adhesion. |
| CD144 | Cell adhesion. |
| CD146 | Cell adhesion. Involved in heterophilic cell to cell |
| | interactions |
| CD147 | Cell adhesion and T-cell activation |
| CD166 | Mediates cell adhesion by binding to CD6 for |
| | intrathymic T-cell development |
| CD171 | Multidomain cell adhesion molecule required for |
| | normal neurohistogenesis |
| CD202b | Signaling, migration and adhesion |
| CD208 | Dendritic cell maturation marker, involved in cell |
| | proliferation |
| CD221 | Receptor for IGF-I and IGF-II. Mediates mitogenic |
| | and anti-apoptotic signals |
| CD227 | Cell adhesion and signaling |
| CD239 | Laminin alpha-5 receptor |
| CD309 | Activation of this receptor by VEGF plays a role in |
| | angiogenesis, development, wound healing, cell |
| | proliferation, migration and differentiation |
| CD321 | Plays a role in epithelial tight junction formation, |
| | regulation of leukocyte erythrocytes. migration, |
| | epithelial barrier maintenance and platelet activation |
| CD324 | Calcium-dependent cell adhesion protein |
| CD326 | A homotypic calcium-independent T-cell adhesion |
| | molecule |
| CD333 | Cell-surface receptor for fibroblast growth factors |
| CD340 | Binds tightly to other ligand-bound EGF receptor |
| | family members to form a heterodimer, stabilizing |
| | ligand binding and enhancing kinase-mediated |
| | activation of downstream signaling pathways |
| CLEC-2 | Hepatocellular and gastric cancers |
| CD147 | Bladder cancer |
| GPVI | Contributes to thrombosis in cancer |

In embodiments, the infectious disease includes a lytic viral infection, a lysogenic viral infection, and/or both.

### CAR-T BioNV Co-Administration

In embodiments, methods of treating and/or preventing a disease or disorder herein include co-administering a whole cell therapy (e.g., T cell, NK cell, TIL, macrophage therapy) with a BioNV. In embodiments, supplementing a whole cell therapy with therapeutic BioNVs/exosomes can be used to decrease the effective dosage of the whole cell therapy needed, reducing CRS, off-target effects, teratoma potential, and the like. In embodiments, methods of treating and/or preventing a disease or disorder include treating cancer, including slowing the growth of a tumor or cancer tissue, reduction of tumor volume, reduction of metastasis, increasing tumor-infiltration lymphocytes (TILs), and/or amelioration of symptoms.

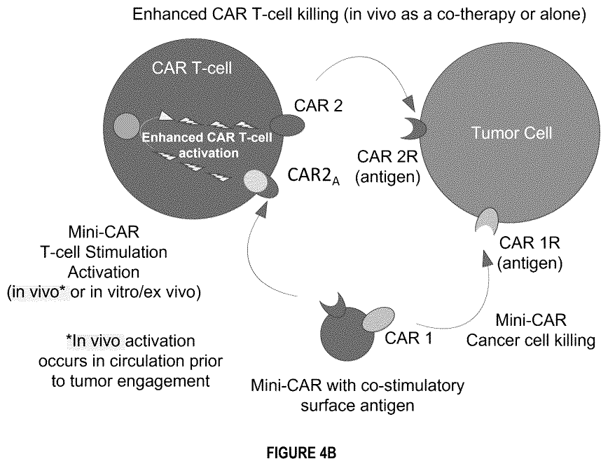

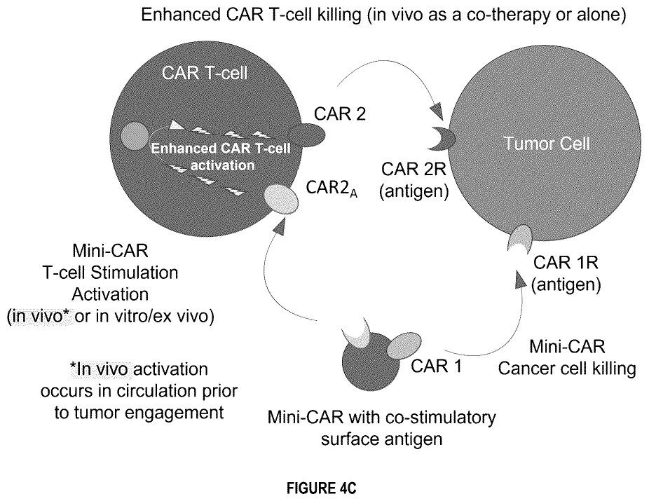

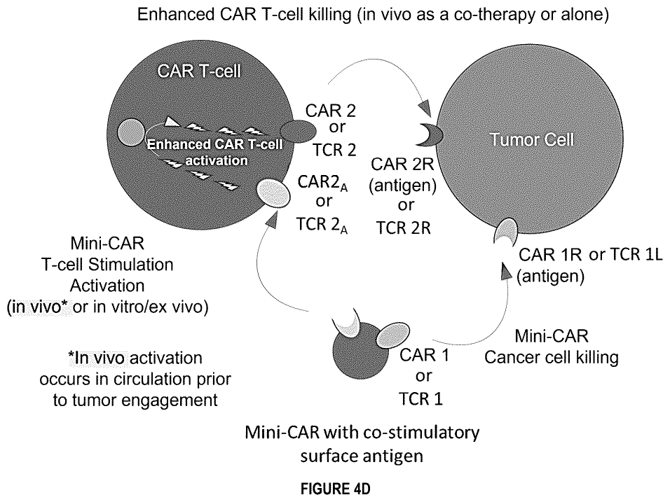

In embodiments, methods of treatment and/or prevention of a disease or disorder herein include administering one or more BioNVs paired with one or more whole cell therapies (e.g., as shown in FIGS. 4A-4C). In embodiments, the pairing is selected as a function of the disease to be treated. In embodiments, the pairing is selected as a function of the desired BioNV—whole cell interaction. In embodiments, the pairing is selected as a function of both the disease to be treated and the nature of the desired interaction between the BioNV and whole cell therapy.

In embodiments, methods of treatment and/or prevention of a disease or disorder include co-administration of a BioNV that has at least one cancer antigen-binding ligand/receptor with a whole cell therapy that has at least one cancer antigen-binding ligand/receptor. In embodiments, the BioNV and whole cell therapy target the same cancer antigen. In embodiments, the BioNV and whole cell therapy target different cancer antigens. Without wishing to be bound by theory, targeting different antigens (i.e., two or more) allows for targeting tumors that present 2 or more biomarkers and/or prevents or diminishes the potential for antigenic escape of a tumor or cancer tissue. For example, without wishing to be bound by theory, a limitation to treating cancer is that the cancer cells could lose expression of an antigen/biomarker that is targeted for treatment.

In embodiments, the BioNV for co-administration includes on its surface an antigen-binding construct (e.g., a CAR) and one or more cancer antigens/biomarkers, or neoantigens derived therefrom. In embodiments the cancer antigens/biomarkers, or neoantigens derived therefrom, is or comprises one or more proteins listed in Table 4, Table 5, and/or Table 6. In embodiments, the BioNV has one or more co-stimulatory molecules (e.g., IL-15) present on the surface for stimulation of a co-administered whole cell (e.g., T cell, NK cell, or macrophage). In embodiments, the BioNV antigen-binding construct does not substantially bind or recognize the antigen/biomarker, or neoantigen derived therefrom, that is also present on the BioNV (e.g., to prevent cross-reactivity).

In embodiments, the BioNV presenting cancer antigens/biomarkers, or neoantigens derived therefrom, is administered as a monotherapy. Without wishing to be bound by theory, in embodiments, the neoantigen-presenting BioNV stimulates a natural T cell response in the subject to be treated via T cell receptor (TCR) engagement, where the natural T cells (now activated in circulation) exhibit improved honing to the site of a tumor or cancer tissue, and exhibits improved cell-mediated cytotoxicity capabilities that would otherwise not exist.

In embodiments, the BioNV presenting cancer antigens/biomarkers, or neoantigens derived therefrom, is co-administered with one of more whole cell therapies. Without wishing to be bound by theory, in embodiments, a BioNV with a CAR and an antigen enables activation/stimulation of a co-administered whole cell therapy (e.g., T cell, NK cell, macrophage, etc.). Without wishing to be bound by theory, in embodiments, whole cells administered to a subject (e.g., CAR-T cells) can become activated by the BioNVs circulating in the blood, which upon activation can improve honing of the whole cell to a tumor or cancer tissue, rather than waiting for the whole cell to become activated in a tumor microenvironment. Without wishing to be bound by theory, in embodiments, cancer antigen/biomarker (or neoantigen) present on a BioNV co-administered with a whole cell counterpart can increase the kinetics (e.g., pharmacokinetics/pharmacodynamics) of tumor cell killing/clearance by the whole cell therapy or BioNV alone.

In embodiments, methods of treatment and/or prevention of a disease or disorder herein include administering a BioNV with a first antigen-binding protein, such as a CAR that recognizes a cancer antigen (e.g., CAR 1 as shown in FIG. 4A), and a second ligand, receptor, and/or antigen that is recognized by a whole cell (e.g., as shown in FIG. 4A). In embodiments, the second ligand, receptor, and/or antigen is the same as, comprises, or resembles a ligand, receptor, and/or antigen expressed by the cancer cell to be targeted (e.g., CAR 2R as shown in FIG. 4A). In embodiments, the second ligand, receptor, and/or antigen on the BioNV is a cancer neoantigen. In embodiments, this BioNV is co-administered with a whole cell that includes an antigen-binding protein, such as a CAR (e.g., CAR 2 as shown in FIG. 4A), where this antigen-binding protein recognizes both the antigen, ligand, or receptor present on the cancer cell, as well as the antigen, ligand, or receptor that is present on the BioNV (e.g., as shown in FIG. 4A). Without wishing to be bound by theory, this treatment strategy improves upon the traditional application of a whole cell therapy by 1) improving the targeting of the whole cell to a tumor cell or cancer tissue by increasing the local concentration of the target antigen, ligand, or receptor, and 2) enhancing whole cell activation/stimulation via the antigen-specific construct due to higher order binding from the combined interaction with both the tumor cell or cancer tissue and the BioNV.

In embodiments, methods of treatment and/or prevention of a disease or disorder herein include administering a BioNV with a first antigen-binding protein, such as a CAR that recognizes a cancer antigen (e.g., CAR 1 as shown in FIG. 4B), and a second ligand, receptor, and/or antigen that is recognized by a whole cell (e.g., as shown in FIG. 4B). In embodiments, the second ligand, receptor, and/or antigen is the same as, comprises, or resembles a ligand, receptor, and/or antigen expressed by the cancer cell to be targeted (e.g., CAR 2R as shown in FIG. 4B). In embodiments, the second ligand, receptor, and/or antigen on the BioNV is a cancer neoantigen. In embodiments, this BioNV is co-administered with a whole cell that includes two or more antigen-binding protein constructs, such as a CAR (e.g., CAR 2 and CAR 2A as shown in FIG. 4B). In embodiments, the whole cell includes a first antigen-binding construct (e.g., CAR) that binds an antigen, ligand, or receptor present on a tumor cell or cancer tissue on the cancer cell, and a second antigen-binding construct (e.g., CAR) binds an antigen, ligand, and/or receptor present on the BioNV (e.g., as shown in FIG. 4B).

In embodiments, the two or more antigen-binding constructs present on the whole cell differ in their intracellular domains—including costimulatory domains and/or intracellular signaling domains, as described herein. In embodiments, the two or more antigen-binding constructs differ in their signaling functionality. For example, in embodiments, the two or more antigen-binding constructs have intracellular domains that signal via different, non-competing signaling pathways, for instance a pro-persistence/pro-survival pathway and a cell-mediated cytotoxicity pathway. In embodiments, the two or more antigen-binding constructs signal through redundant, or overlapping, signaling pathways. Without wishing to be bound by theory, this treatment strategy improves upon the traditional administration of a whole cell therapy by, without limitation, 1) improving the targeting of the whole cell to a tumor cell or cancer tissue by increasing the local concentration of the target antigen, ligand, or receptor, 2) enhancing whole cell activation/stimulation via the antigen-specific construct due to higher order binding from the combined interactions with the tumor cell or cancer tissue and the BioNV, and 3) enhancing whole cell activation/stimulation via the two or more different antigen-specific constructs having different intracellular domains.

In embodiments, methods of treatment and/or prevention of a disease or disorder herein include administering a BioNV with a first antigen-binding protein, such as a CAR (e.g., CAR 1 as shown in FIG. 4C), and a second ligand, receptor, and/or antigen that is recognized by a whole cell (e.g., as shown in FIG. 4C). In embodiments, the second ligand, receptor, and/or antigen is not the same as, does not comprise, or does not resemble a ligand, receptor, and/or antigen expressed by the cancer cell to be targeted (e.g., CAR 2R as shown in FIG. 4C). In embodiments, the second ligand, receptor, and/or antigen present on the BioNV—to be recognized by the whole cell—is selected for a specific cell-BioNV interaction that is not intended to appreciably occur elsewhere in the subject to be treated. In embodiments, the BioNV is co-administered with a whole cell that includes at least two different antigen-binding constructs, such as a CAR that binds an antigen, ligand, or receptor present on a cancer cell (e.g., CAR 2 as shown in FIG. 4C) and a CAR that binds the BioNV (e.g., CAR 2A as shown in FIG. 4C). In embodiments, the whole cell has a first antigen-binding construct that binds an antigen, ligand, or receptor present on a tumor cell or cancer tissue, and a second antigen-binding construct that binds a ligand, receptor, and/or antigen that is present on the BioNV (e.g., as shown in FIG. 4C), wherein the ligand, receptor, and/or antigen on the BioNV is, different from the antigen, ligand, or receptor present on the tumor cell or cancer tissue.

In embodiments, the first antigen-binding construct and the second antigen-binding construct present on the whole cell differ in their intracellular domains—including costimulatory domains and/or intracellular signaling domains, as described herein. In embodiments, the two antigen-binding constructs differ in their signaling functionality. For example, in embodiments, the whole cell has a first antigen-binding construct that binds a tumor cell or cancer tissue (e.g., CAR 2-CAR 2R as shown in FIG. 4C) that comprises intracellular domains that signal via cell-mediated cytotoxicity signaling pathways (e.g., for cellular release of granzyme, perforin, among other antitumor cytokines). For example, in embodiments, the whole cell has a second antigen-binding construct that binds a BioNV (e.g., CAR 2A as shown in FIG. 4C) that comprises intracellular domains that signal via different, non-cell-mediated cytotoxicity signaling pathways, e.g., such as a pro-persistence, pro-survival, and/or pro-proliferative pathway. Without wishing to be bound by theory, this treatment strategy improves upon the traditional application of a whole cell therapy by, without limitation, 1) improving the targeting of the whole cell to a tumor cell or cancer tissue by increasing the local concentration of the target antigen, ligand, or receptor, 2) enhancing whole cell activation/stimulation via the antigen-specific construct due to higher order binding from the combined interactions with the tumor cell or cancer tissue and the BioNV, 3) enhancing whole cell activation/stimulation via the two or more different intracellular domains, and 4) reducing peripheral, or off-target activation/stimulation of the whole cell by requiring both the tumor cell or cancer tissue interaction, as well as the BioNV interaction, for full function of the whole cell.

In embodiments, a T cell receptor (TCR) can be used in place of, or in addition to the CAR constructs described herein (e.g., as shown in FIG. 40). In embodiments, the TCR is a native TCR originating from a subject intended to be treated. In embodiments, the TCR is an engineered T cell receptor.

In embodiments, methods of treatment and/or prevention of a disease or disorder include co-administration include first administering an effective amount (e.g., one or more doses) of a BioNV prior to administration of an effective amount (e.g., one or more doses) of a whole cell therapy. In embodiments, the BioNV is first administered about or at least about 2 hours before the whole cell therapy, about or at least about 4 hours before the whole cell therapy, about or at least about 6 hours before the whole cell therapy, about or at least about 8 hours before the whole cell therapy, about or at least about 12 hours before the whole cell therapy, about or at least about 24 hours before the whole cell therapy, about or at least about 2 days before the whole cell therapy, about or at least about 3 days before the whole cell therapy, about or at least about 4 days before the whole cell therapy, about or at least about 5 days before the whole cell therapy, about or at least about 6 days before the whole cell therapy, or about or at least about 1 week or more before the whole cell therapy. In embodiments, the BioNV is first administered to decorate cancer cells prior to administering a whole cell therapy.

In embodiments, methods of treatment and/or prevention of a disease or disorder include administering the BioNV and whole cell therapy are administering contemporaneously.

In embodiments, methods of treatment and/or prevention of a disease or disorder include co-administration include first administering an effective amount (e.g., one or more doses) of a whole cell prior to administration of an effective amount (e.g., one or more doses) of a BioNV. In embodiments, the whole cell is first administered about or at least about 2 hours before the BioNV, about or at least about 4 hours before the BioNV, about or at least about 6 hours before the BioNV, about or at least about 8 hours before the BioNV, about or at least about 12 hours before the BioNV, about or at least about 24 hours before the BioNV, about or at least about 2 days before the BioNV, about or at least about 3 days before the BioNV, about or at least about 4 days before the BioNV, about or at least about 5 days before the BioNV, about or at least about 6 days before the BioNV, or about or at least about 1 week or more before the BioNV. In embodiments, the whole cell therapy is first administered due to the relative ease of BioNVs for tissue/tumor penetrance.

In embodiments, the whole cell is a whole cell therapy that includes one or more of a CAR-T cell, CAR-NK cell, and/or CAR-macrophage cell therapy. In embodiments, the whole cell therapy is one or more of: ABECMA (idecabtagene vicleucel, Celgene Corporation, Bristol-Myers Squibb), ADSTILADRIN (nadofaragene firadenovec, Ferring Pharmaceuticals A/S), BREYANZI (lisocabtagene maraleucel, Juno Therapeutics, Inc., Bristol-Myers Squibb), CARVYKTI (ciltacabtagene autoleucel, Janssen Biotech, Inc.), GINTUIT (Allogeneic Cultured Keratinocytes and Fibroblasts in Bovine Collagen, Organogenesis Inc.), HEMGENIX (etranacogene dezaparvovec, CSL Behring LLC), HPC (Cord Blood, multiple sources), IMLYGIC (talimogene laherparepvec, BioVex, Inc., Amgen Inc.), KYMRIAH (tisagenlecleucel, Novartis Pharmaceuticals Corporation), LAVIV (Azficel-T, Fibrocell Technologies), LUXTURNA (voretigene neparvovec, Spark Therapeutics, Inc.), MACI (Autologous Cultured Chondrocytes on a Porcine Collagen Membrane, Vericel Corp.), PROVENGE (sipuleucel-T, Dendreon Corp.), RETHYMIC (allogeneic processed thymus tissue, Enzyvant Therapeutics GmbH), SKYSONA (elivaldogene autotemcel, bluebird bio, Inc.), STRATAGRAFT (allogenic cultured keratinovytes and dermal fibroblasts in murine collagen, Stratatech Corporation, TECARTUS (brexucabtagene autoleucel, Kite Pharma, Inc.), YESCARTA (axicabtagene ciloleucel, Kite Pharma, Inc.), ZYNTEGLO (betibeglogene autotemcel, Bluebird Bio, Inc.), ZOLGENSMA (onasemnogene abeparvovec-xioi; Novartis Gene Therapies, Inc.). In embodiments, the whole cell therapy is one or more of the whole cell therapies described herein that has been engineered to include one or more BioNV-compatible cell surface proteins, as described herein. In embodiments, the whole cell therapy is one or more of the whole cell therapies described herein that has been engineered to be a hypoimmunogenic cell, as described herein.

In embodiments, the administration of the BioNV in addition to the administration of the whole cell improves the functionality of whole cell, relative to administration of the whole cell alone. The functionality of the whole cell, in embodiments, includes one or more of cell-mediated cytotoxicity, cytokine release, tumor cell or cancer cell killing, honing to a tumor cell or cancer tissue, tissue infiltration, proliferation, persistence, and/or survival. In embodiments, the BioNV reduces one or more toxicities of the whole cell. In embodiments, toxicities of whole cell therapies include propensity for off-target binding (e.g., binding in peripheral tissues, non-specific activation, etc.) and associated adverse events.

In embodiments, methods of treating and/or preventing a disease or disorder include administering an additional therapeutic agent. In embodiments, the additional therapeutic agent can be any additional anti-cancer agent, anti-infective agent, analgesic, and/or non-steroidal inflammatory agent (NSAID).

In embodiments, therapeutic BioNVs/exosomes can be frozen at −80° C., or suitable for storage at about −80° C., and/or lyophilized (e.g., for reconstitution in buffer). In embodiments, therapeutic BioNVs/exosomes can be stable at about ambient temperature, at about −20° C., at about 4° C., at about 25° C., or at about 37° C. for at least about 1 hour, at least about 2 hours, at least about 4 hours, at least about 6 hours, at least about 12 hours, at least about 24 hours, at least about 2 days, at least about one week, or at least about one month or longer.

In embodiments, treating and/or preventing a disease or disorder can be achieved within about 2 weeks, within about 4 weeks, within about 6 weeks, within about 12 weeks, within about 18 weeks, within about 24 weeks, within about 6 months, within about 1 year, or within about 2 or more years from administration of the composition and methods with such compositions.

### Dosing and Administration

The dosage of any therapeutic BioNVs/exosomes disclosed herein as well as the dosing schedule can depend on various parameters and factors, including, but not limited to, the specific therapeutic BioNVs/exosomes, the disease being treated, the severity of the condition, whether the condition is to be treated or prevented, the subject's age, weight, and general health, and the administering physician's discretion. Additionally, pharmacogenomic (the effect of genotype on the pharmacokinetic, pharmacodynamic or efficacy profile of a therapeutic) information about a particular subject may affect dosage used. Furthermore, the exact individual dosages can be adjusted somewhat depending on a variety of factors, including the specific combination of the agents being administered, the time of administration, the route of administration, the nature of the formulation, the rate of excretion, the disease being treated, the severity of the disorder, and the anatomical location of the disorder. Some variations in the dosage can be expected.

In embodiments, delivery of therapeutic BioNVs/exosomes can be like that of a vesicle, in particular a liposome (see Langer, 1990, Science 249:1527-1533; Treat et al., in Liposomes in Therapy of Infectious Disease and Cancer, Lopez-Berestein and Fidler (eds.), Liss, New York, pp. 353-365 (1989)).

Methods of treating and/or preventing a disease or disorder using therapeutic BioNVs/exosomes described herein, in embodiments, include dosage ranges in concentration of number of therapeutic BioNVs/exosomes per kilogram (kg) subject body weight. In embodiments, suitable dosage ranges for methods described herein can include from about 10³ BioNVs/kg (or exosomes/kg) to about 10¹² BioNVs/kg (or exosomes/kg). In embodiments, the therapeutic BioNVs/exosomes are present in the composition at a concentration of about 10³ BioNVs/mL (or exosomes/mL) to about 10¹⁴ BioNVs/mL (or exosomes/mL). Alternatively, in embodiments, therapeutic BioNV/exosome compositions are present in compositions as weight/volume in the range of about 5 ng/mL to about 500 mg/mL. In embodiments, the therapeutic BioNV/exosome dosages are based on the size of the therapeutic BioNVs/exosomes used for the treatment, for example, therapeutic BioNVs/exosomes at 1000 nm are provided in approximately 5-fold to 10-fold fewer amounts than 100 nm BioNVs/exosomes for a comparable dose.

In embodiments, therapeutic BioNVs/exosomes disclosed herein are administered by a controlled-release or a sustained-release means or by delivery of a device that is well known to those of ordinary skill in the art. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,556, each of which is incorporated herein by reference in its entirety. Such dosage forms can be useful for providing controlled or sustained-release of one or more active ingredients using, for example, hydropropylmethyl cellulose, other polymer matrices, gels, permeable membranes, osmotic systems, multilayer coatings, microparticles, microspheres, or a combination thereof, to provide the desired release profile in varying proportions. Controlled- or sustained-release of an active ingredient can be stimulated by various conditions, including but not limited to, changes in pH, changes in temperature, stimulation by an appropriate wavelength of light, concentration or availability of enzymes, concentration or availability of water, or other physiological conditions or compounds.

In embodiments, polymeric materials are used (see Medical Applications of Controlled Release, Langer and Wise (eds.), CRC Pres., Boca Raton, Florida (1974); Controlled Drug Bioavailability, Drug Product Design and Performance, Smolen and Ball (eds.), Wiley, New York (1984); Ranger and Peppas, 1983, J. Macromol. Sci. Rev. Macromol. Chem. 23:61; Levy et al., 1985, Science 228:190; During et al., 1989, Ann. Neurol. 25:351; Howard et al., 1989, J. Neurosurg. 71:105).

In embodiments, a controlled-release system is placed in proximity of the target area to be treated, thus requiring only a fraction of the systemic dose (see, e.g., Goodson, in Medical Applications of Controlled Release, supra, vol. 2, pp. 115-138 (1984)). Other controlled-release systems discussed in the review by Langer, 1990, Science 249:1527-1533 may be used.

In embodiments, the methods using therapeutic BioNVs/exosomes include applying therapeutic BioNVs/exosomes to a surface of a device (e.g., a catheter) or contained within a pump, patch, or other drug delivery device. The excipient or carrier can be selected based on the mode and route of administration. Suitable pharmaceutical carriers, as well as pharmaceutical necessities for use in pharmaceutical formulations, are described in Remington's Pharmaceutical Sciences (E. W. Martin), a well-known reference text in this field, and in the USP/NF (United States Pharmacopeia and the National Formulary).

In embodiments, therapeutic BioNVs/exosomes can be administered at doses that are congruent to dosages of whole cells, for example, based on CAR concentration. In embodiments, the typical concentration range of CAR protein per microgram of T cells is between 0.20 ng-0.70 ng, whereas a single BioNV may have a total number of CARs that is 5 times to 10,000 times less than the whole cell, resulting in a conversion of BioNV mass to CAR concentration, where the CAR concentration can be assumed equivalent (such as the case in exosomes) or increased (such as the case in BioNVs) to the cell from which it originated (e.g., the T cell). In embodiments, the concentration and/or surface density of the targeting agent (e.g., CAR) is increased on the BioNV compared to the whole cell from which is it derived. In embodiments, the concentration and/or surface density of the targeting agent (e.g., CAR) is enriched by serial extrusion processing of the whole cell. In embodiments, the concentration and/or surface density of the targeting agent (e.g., CAR), among other cell surface molecules, on the BioNV is 2-fold to 100-fold increased relative to the whole cell. In embodiments, due to exosomes being naturally shed, their concentration and/or surface density of the targeting agent (e.g., CAR), among other cell surface molecules, is substantially the same as the whole cell.

The dosage regimen utilizing any therapeutic BioNVs/exosomes disclosed herein can be selected in accordance with a variety of factors including cancer type, species, age, weight, sex, and medical condition of the subject; the severity of the condition to be treated; the route of administration; the renal or hepatic function of the subject; the pharmacogenomic makeup of the individual; and the specific composition of the disclosure employed. Any therapeutic BioNVs/exosomes disclosed herein can be administered in a single daily dose, or the total daily dosage can be administered in divided doses of two, three, or four times daily. Furthermore, any therapeutic BioNVs/exosomes disclosed herein can be administered continuously rather than intermittently throughout the dosage regimen.

In embodiments, therapeutic BioNVs/exosomes are administered in consecutive doses about every hour, about every 2 hours, about every 6 hours, about every 12 hours, about every 24 hours, about every 2 days, about every 4 days, about every 7 days, about every 2 weeks, about every 4 weeks, or about every month.

### Additional Therapeutic Agents

In embodiments, the compositions or methods described herein further comprise a therapeutically effective amount of one or more additional therapeutic agents. In embodiments, the therapeutically effective amount of one or more additional therapeutic agents may be in solution with a therapeutic BioNV/exosome, adsorbed onto the surface of the NV, or a payload encapsulated within a therapeutic BioNV/exosome. In embodiments, the additional therapeutic agent is one or more of a checkpoint inhibitor, an analgesic, and/or an anti-infective agent.

In embodiments, the present compositions or methods contemplate other additional therapeutic agents, for example, an analgesic, to aid in treating inflammation or pain at the site of the administration, or an anti-infective agent to prevent infection of the site of treatment with the composition. Non-limiting examples of additional therapeutic agents include analgesics, such as nonsteroidal anti-inflammatory drugs, opiate agonists and salicylates; anti-infective agents, such as anthelmintics, antianaerobics, antibiotics, aminoglycoside antibiotics, antifungal antibiotics, cephalosporin antibiotics, macrolide antibiotics, miscellaneous B-lactam antibiotics, penicillin antibiotics, quinolone antibiotics, sulfonamide antibiotics, tetracycline antibiotics, antimycobacterials, antituberculosis antimycobacterials, antiprotozoals, antimalarial antiprotozoals, antiviral agents, anti-retroviral agents, scabicides, anti-inflammatory agents, corticosteroid anti-inflammatory agents, antipruritics/local anesthetics, topical anti-infectives, antifungal topical anti-infectives, antiviral topical anti-infectives; electrolytic and renal agents, such as acidifying agents, alkalinizing agents, diuretics, carbonic anhydrase inhibitor diuretics, loop diuretics, osmotic diuretics, potassium-sparing diuretics, thiazide diuretics, electrolyte replacements, and uricosuric agents; enzymes, such as pancreatic enzymes and thrombolytic enzymes; gastrointestinal agents, such as antidiarrheals, antiemetics, gastrointestinal anti-inflammatory agents, salicylate gastrointestinal anti-inflammatory agents, antacid anti-ulcer agents, gastric acid-pump inhibitor anti-ulcer agents, gastric mucosal anti-ulcer agents, H2-blocker anti-ulcer agents, cholelitholytic agents, digestants, emetics, laxatives and stool softeners, and prokinetic agents; general anesthetics, such as inhalation anesthetics, halogenated inhalation anesthetics, intravenous anesthetics, barbiturate intravenous anesthetics, benzodiazepine intravenous anesthetics, and opiate agonist intravenous anesthetics; hormones and hormone modifiers, such as abortifacients, adrenal agents, corticosteroid adrenal agents, androgens, anti-androgens; immunobiological agents, such as immunoglobulins, immunosuppressives, toxoids, and vaccines; local anesthetics, such as amide local anesthetics and ester local anesthetics; musculoskeletal agents, such as anti-gout anti-inflammatory agents, corticosteroid anti-inflammatory agents, gold compound anti-inflammatory agents, immunosuppressive anti-inflammatory agents, nonsteroidal anti-inflammatory drugs (NSAIDs), salicylate anti-inflammatory agents; minerals; vitamins, such as water soluble or fat soluble vitamins, vitamin A, vitamin B, vitamin C, vitamin D, vitamin E, and/or vitamin K; and radionuclides such as Yttrium-90, Iodine-131, Samarium-153, Lutetium-177, Astatine-211, Lead-212/bismuth-212, Radium-223, Actinium-225, and Thorium-227.

Additional non-limiting examples of useful therapeutic agents from the above categories include: (1) analgesics in general, such as lidocaine or derivatives thereof, and NSAID analgesics, including diclofenac, ibuprofen, ketoprofen, and naproxen; (2) opiate agonist analgesics, such as codeine, fentanyl, hydromorphone, and morphine; (3) salicylate analgesics, such as aspirin (ASA) (enteric coated ASA); (4) Hi-blocker antihistamines, such as clemastine and terfenadine; (5) anti-infective agents, such as mupirocin; (6) antianaerobic anti-infectives, such as chloramphenicol and clindamycin; (7) antifungal antibiotic anti-infectives, such as amphotericin b, clotrimazole, fluconazole, and ketoconazole; (8) macrolide antibiotic anti-infectives, such as azithromycin and erythromycin; (9) miscellaneous B-lactam antibiotic anti-infectives, such as aztreonam and imipenem; (10) penicillin antibiotic anti-infectives, such a s nafcillin, oxacillin, penicillin G, and penicillin V; (11) quinolone antibiotic anti-infectives, such as ciprofloxacin and norfloxacin; (12) tetracycline antibiotic anti-infectives, such as doxycycline, minocycline, and tetracycline; (13) antituberculosis antimycobacterial anti-infectives such as isoniazid (INH), and rifampin; (14) antiprotozoal anti-infectives, such as atovaquone and dapsone; (15) antimalarial antiprotozoal anti-infectives, such as chloroquine and pyrimethamine; (16) anti-retroviral anti-infectives, such as ritonavir and zidovudine; (17) antiviral anti-infective agents, such as acyclovir, ganciclovir, interferon alfa, remdesivir, and rimantadine; (18) antifungal topical anti-infectives, such as amphotericin B, clotrimazole, miconazole, and nystatin; (19) antiviral topical anti-infectives, such as acyclovir; (20) electrolytic and renal agents, such as lactulose; (21) loop diuretics, such as furosemide; (22) potassium-sparing diuretics, such as triamterene; (23) thiazide diuretics, such as hydrochlorothiazide (HCTZ); (24) uricosuric agents, such as probenecid; (25) enzymes such as RNase and DNase; (26) antiemetics, such as prochlorperazine; (27) salicylate gastrointestinal anti-inflammatory agents, such as sulfasalazine; (28) gastric acid-pump inhibitor anti-ulcer agents, such as omeprazole; (29) H2-blocker anti-ulcer agents, such as cimetidine, famotidine, nizatidine, and ranitidine; (30) digestants, such as pancrelipase; (31) prokinetic agents, such as erythromycin; (32) ester local anesthetics, such as benzocaine and procaine; (33) musculoskeletal corticosteroid anti-inflammatory agents, such as beclomethasone, betamethasone, cortisone, dexamethasone, hydrocortisone, and prednisone; (34) musculoskeletal anti-inflammatory immunosuppressives, such as azathioprine, cyclophosphamide, and methotrexate; (35) musculoskeletal nonsteroidal anti-inflammatory drugs (NSAIDs), such as diclofenac, ibuprofen, ketoprofen, ketorlac, and naproxen; (36) minerals, such as iron, calcium, and magnesium; (37) vitamin B compounds, such as cyanocobalamin (vitamin B12) and niacin (vitamin B3); (38) vitamin C compounds, such as ascorbic acid; and (39) vitamin D compounds, such as calcitriol.

### Compositions of Therapeutic BioNVs/Exosomes

In aspects, the present disclosure relates to compositions that can be used for the treatment of mammalian diseases comprising an allogeneic, hypoimmunogenic therapeutic BioNV/exosome comprising one or more therapeutically relevant biomolecules, as described herein.

In embodiments, compositions include therapeutic BioNVs/exosomes. In embodiments, compositions can include a therapeutic BioNV/exosome and at least one or more of an anti-cancer therapeutic, anti-infective therapeutic, or gene editing payload. In embodiments, composition include a therapeutic BioNVs/exosomes which can adsorb therapeutic molecules onto the surface of the NV and/or encapsulate a therapeutic payload within an aqueous compartment of the NV. In embodiments, the composition comprises a therapeutically effective amount of the BioNVs/exosomes.

In embodiments, the composition is allogeneic and/or hypoimmunogenic. In embodiments, the composition is derived from iPSCs (among other cell types) which have been modified to reduce expression of immunogenic molecules and/or increase expression of immunoprotective molecules.

In embodiments, the composition of a therapeutic BioNV/exosome is hypoimmunogenic. For example, in embodiments, the composition does not result in an inflammatory reaction and/or an immune response upon administration. In embodiments, the therapeutic BioNVs/exosomes are hypoimmunogenic. In embodiments, upon administration to a subject, the composition, optionally the therapeutic BioNVs/exosomes therein, elicits less than about 50%, about 45%, about 40%, about 35%, about 30%, about 25%, about 24%, about 23%, about 22%, about 21%, about 20%, about 19%, about 18%, about 17%, about 16%, about 15%, about 14%, about 13%, about 12%, about 11%, about 10%, about 9%, about 8, about 7%, about 6%, about 5%, about 4%, about 3%, about 2%, or about 1% of an inflammatory or immune response measured as a function of cytokine, chemokine, or immunomodulatory enzyme concentration, such as IL-1, IL-2, IL-3, IL-4, IL-6, IL-7, IL-9, IL-10, IL-11, IL-12, IL-13, IL-14, IL-15, IL-16, IL-17, IL-20, IFN-α/β/γ, TNFα/β, IDO, HLA-G, HGF, PGE2, among others, or any combination thereof, in comparison, e.g. to a cognate whole cell therapy counterpart.

In embodiments, the therapeutic BioNVs/exosomes are present in the composition at a concentration of about 10³ BioNVs/mL (or exosomes/mL) to about 10¹⁴ BioNVs/mL (or exosomes/mL). Alternatively, in embodiments, therapeutic BioNV/exosome compositions are present in compositions as weight/volume in the range of about 5 ng/mL to about 500 mg/mL.

In embodiments, the composition is substantially free of one or more bacteria, virus, fungus, spore, mycoplasma, pyrogen, and in more particular embodiments, is substantially free of all the foregoing. In embodiments, the composition is substantially free of whole cells and intracellular cell components including organelles such as nuclei, mitochondria, Golgi, etc., and/or substantially free of non-CAR-expressing NVs and/or substantially free of ruptured, damaged NVs. In embodiments, the composition is substantially free of cellular chromatin, nucleosomes, and other genetic material and non-therapeutic nucleic acids. In embodiments, BioNV/exosomes and BioNV/exosome compositions are substantial free of cellular genomic DNA.

In embodiments, therapeutic BioNVs/exosomes are modular and allogeneic (off-the-shelf) due to the lack of immunogenicity from engineered iPSCs. In embodiments, the lack of whole cell signaling components allows BioNVs/exosomes to be easily tunable for target specificity and resistance to immunosuppressive signals. In embodiments, therapeutic BioNVs/exosomes lack the genetic elements that contribute to runaway cytokine storms, minimizing patient risk of cytokine release syndrome (CRS). In embodiments, the amounts of active cytokine, perforin, granzymes, granulysin, interferon, interleukins, etc., encapsulated within the therapeutic BioNV/exosome are regulated during upstream (pre-BioNV/exosome derivation) cellular processes. In embodiments, therapeutic BioNVs/exosomes are derived from cells capable of crossing biological barriers and/or viral receptors known for facilitation crossing.

Without wishing to be bound by theory, therapeutic BioNVs/exosomes generated from iPSC engineered allogeneic base cell lines represent immune invisible, meaning that therapeutic BioNVs/exosomes have the potential for multi-dosing, therapeutic BioNV/exosome antibody-mediated neutralization is minimized, and immune cell-mediated clearance is evaded (T cell and macrophage). In embodiments, therapeutic BioNVs/exosomes do not contain viable genetic material from the cells they were derived to cause CRS or teratoma. In embodiments, increased expression of certain cytokines encapsulated within a therapeutic BioNV/exosome can recruit natural T cells. In embodiments, BioNVs/exosomes can be derived from modified cell types with or without barrier penetrating ligands to further control activity post-infusion.

### Pharmaceutical Compositions and Formulations of Therapeutic BioNVs/Exosomes

In aspects, the composition is a pharmaceutical composition. In embodiments, the pharmaceutical compositions of the present disclosure are formulated to provide a therapeutically effective amount of therapeutic BioNVs/exosomes as the active ingredient. In embodiments, the pharmaceutical compositions of the present disclosure are formulated to provide a therapeutically effective amount of one or more anticancer therapeutics as a payload within a therapeutic BioNV/exosome as the active ingredient. Typically, the pharmaceutical compositions also comprise one or more pharmaceutically acceptable excipients, carriers, including inert solid diluents and fillers, diluents, including sterile aqueous solution and various organic solvents, permeation enhancers, solubilizers and adjuvants.

Pharmaceutical excipients can be liquids, such as water and oils, including those of petroleum, animal, vegetable, or synthetic origin, such as peanut oil, soybean oil, mineral oil, sesame oil and the like. The pharmaceutical excipients can be, for example, saline, gum acacia, gelatin, starch paste, talc, keratin, colloidal silica, urea, and the like. In addition, auxiliary, stabilizing, thickening, lubricating, and coloring agents can be used. Pharmaceutically acceptable excipients are generally sterile when administered to a subject. Water is a useful excipient when any agent disclosed herein is administered intravenously. Saline solutions and aqueous dextrose and glycerol solutions can also be employed as liquid excipients, specifically for injectable solutions. Suitable pharmaceutical excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. Any composition disclosed herein, if desired, can also formulated with wetting or emulsifying agents, or pH buffering agents. Other examples of suitable pharmaceutical excipients are described in Remington's Pharmaceutical Sciences 1447-1676 (Alfonso R. Gennaro eds., 19th ed. 1995), incorporated herein by reference.

In embodiments, the composition comprises an excipient or carrier. In embodiments, the diluent is a pharmaceutically acceptable excipient or carrier.

In embodiments, the pharmaceutical composition comprises a pharmaceutically acceptable diluent. Non-limiting example of diluents include liquid diluents such as water, ethanol, propylene glycol, glycerin, and various combinations thereof, and inert solid diluents such as calcium carbonate, calcium phosphate or kaolin. In embodiments, the diluent comprises one or more of saline, phosphate buffered saline, Dulbecco's Modified Eagle Medium (DMEM), alpha modified Minimal Essential Medium (alpha MEM), Roswell Park Memorial Institute Media 1640 (RPMI Media 1640), HBSS, human albumin, Ringer's solution, and the like, or any combination thereof.

In embodiments, the active ingredient is typically mixed with an excipient, diluted by an excipient or enclosed within such a carrier in the form of, for example, a capsule, tablet, sachet, paper, or other container. When the excipient serves as a diluent, it can be a solid, semisolid, or liquid material (e.g., normal saline), which acts as a vehicle, carrier or medium for the active ingredient. In embodiments, the compositions can be in the form of tablets, pills, powders, lozenges, sachets, cachets, elixirs, suspensions, emulsions, solutions, syrups, aerosols (as a solid or in a liquid medium), lotions, creams, ointments, gels, soft and hard gelatin capsules, suppositories, sterile injectable solutions, and sterile packaged powders. As is known in the art, the type of diluent can vary depending upon the intended route of administration. In embodiments, the resulting compositions can include additional agents, such as preservatives, cryopreservatives (e.g., DMSO), and/or lyoprotectants (e.g., polyols, salts). In embodiments, the carrier can be, or can include a lipid-based or polymer-based colloid. In embodiments, the carrier material can be a colloid formulated as a liposome, a hydrogel, a microparticle, a nanoparticle, or a block copolymer micelle. In embodiments, the carrier material can form a capsule, and that material may be a polymer-based colloid.

In embodiments, the pharmaceutical compositions comprising the therapeutic BioNVs/exosomes include a solubilizing agent. In embodiments, the pharmaceutical compositions comprising the therapeutic BioNVs/exosomes include a cryoprotective agent or an agent to improve thermal stability, such as DMSO or glycerol. The pharmaceutical compositions, in embodiments, can be delivered with a suitable vehicle or delivery device as known in the art.

In embodiments, the composition comprises a scaffold of biomaterials. In a non-limiting example, the three-dimensional biomaterials include therapeutic BioNVs/exosomes embedded in an extracellular matrix attached to, or dispersed within, or trapped within the scaffold. In embodiments, the biomaterials are biodegradable and/or synthetic.

In embodiments, the scaffold comprises biodegradable biomaterials. Non-limiting examples of biodegradable biomaterials include fibrin, collagen, elastin, gelatin, vitronectin, fibronectin, laminin, reconstituted basement membrane matrix, starch, dextran, alginate, hyaluron, chitin, chitosan, agarose, sugars, hyaluronic acid, poly (lactic acid), poly (glycolic acid), polyethylene glycol, decellularized tissue, self-assembling peptides, polypeptides, glycosaminoglycans, derivatives and mixtures thereof. Other useful biodegradable polymers or polymer species include, but are not limited to, polydioxanone, polycarbonate, polyoxalate, poly (α-ester), polyanhydride, polyacetate, polycaprolactone, poly (ortho Esters), polyamino acids, polyamides, and mixtures and copolymers thereof, L-lactic acid and D-lactic acid stereopolymers, copolymers of bis (para-carboxyphenoxy) propanoic acid and sebacic acid, sebacic acid copolymers, caprolactone Copolymer, poly (lactic acid)/poly (glycolic acid)/polyethylene glycol copolymer, polyurethane and poly (lactic acid) copolymer, polyurethane and poly (lactic acid) copolymer, α-amino acid copolymer, α-amino acid and caproic acid copolymer, A-benzylglutamate and polyethylene glycol copolymers, succinate and poly (glycol) copolymers, polyphosphazenes, polyhydroxy-alkanoates and mixtures thereof. Binary and ternary systems are also contemplated. In embodiments, the scaffold comprises one or more of collagen, various proteoglycans, alginate-based substrates, and chitosan. In embodiments, the scaffold comprises one or more of a hydrogel, silk, Matrigel, acellular and/or decellarized scaffolds, poly-ε-caprolactone scaffolds, resorbable scaffolds, and nanofiber-hydrogel composite.

In embodiments, the scaffold comprises synthetic biomaterials. Non-limiting examples of synthetic biomaterials include lactone-based polyesters or copolyesters such as polylactide, polycaprolactonglycolide, polyorthoesters, polyanhydrides, polyaminoacids, polysaccharides, polyphosphazenes, poly(ether-ester) copolymers (e.g., PEO-PLLA); polydimethylsiloxane, poly(ethylene-vinylacetate), acrylate-based polymers or copolymers (e.g., polyhydroxyethyl methylmethacrylate, polyvinyl pyrrolidinone), fluorinated polymers such as polytetrafluoroethylene and cellulose esters.

In embodiments, the compositions can be prepared in any manner well known in the pharmaceutical arts, and can be administered by a variety of routes (e.g., subcutaneous, intravenous, etc.) depending upon whether local or systemic treatment is desired and upon the area to be treated. In embodiments, administration can be topical (including ophthalmic and to mucous membranes including intranasal, vaginal, and rectal delivery), pulmonary (e.g., by inhalation or insufflation of powders or aerosols, including by nebulizer; intratracheal, intranasal, epidermal and transdermal), ocular, oral, or parenteral. In embodiments, methods can include ocular delivery, topical administration (eye drops), subconjunctival, periocular or intravitreal injection or introduction by balloon catheter or ophthalmic inserts surgically placed in the conjunctival sac. In embodiments, parenteral administration includes intravenous, intra-arterial, subcutaneous, intraperitoneal or intramuscular injection or infusion; or intracranial, e.g., intrathecal or intraventricular administration. In embodiments, parenteral administration can be in the form of a single bolus dose, or may be, for example, by a continuous perfusion pump.

In embodiments, pharmaceutical compositions and formulations for topical administration may include transdermal patches, ointments, lotions, creams, gels, drops, suppositories, sprays, liquids, powders, and the like. In embodiments, methods of treating and/or preventing cancer include the use of pharmaceutical carriers, aqueous, powder or oily bases, thickeners, and the like.

In embodiments, the pharmaceutical compositions contain, as the active ingredient, nucleic acids and vectors described herein in combination with one or more pharmaceutically acceptable carriers. In embodiments, the terms “pharmaceutically acceptable” (or “pharmacologically acceptable”) refer to molecular entities and compositions that do not produce an adverse, allergic or other untoward reaction, when administered to an animal or a human, as appropriate. The methods and compositions disclosed herein can be applied to a wide range of species, e.g., humans, non-human primates (e.g., monkeys), horses or other livestock, dogs, cats, ferrets or other mammals kept as pets, rats, mice, or other laboratory animals. In embodiments, the term “pharmaceutically acceptable carrier,” includes any and all solvents, dispersion media, coatings, antibacterial, isotonic and absorption delaying agents, buffers, excipients, binders, lubricants, gels, surfactants and the like, that may be used as media for a pharmaceutically acceptable substance.

In embodiments, the compositions can be applied to a surface of a device (e.g., a catheter) or contained within a pump, patch, or other drug delivery device. In embodiments, the compositions can be administered alone, or in a mixture, in the presence of a pharmaceutically acceptable excipient or carrier (e.g., physiological saline). The excipient or carrier is selected based on the mode and route of administration. Suitable pharmaceutical carriers, as well as pharmaceutical necessities for use in pharmaceutical formulations, are described in Remington's Pharmaceutical Sciences (E. W. Martin), a well-known reference text in this field, and in the USP/NF (United States Pharmacopeia and the National Formulary).

In embodiments, the compositions, e.g., pharmaceutical compositions, disclosed herein are suspended in a saline buffer (including, without limitation, TBS, PBS, and the like).

The present technology includes the disclosed therapeutic BioNVs/exosomes in various formulations of pharmaceutical compositions. Therapeutic BioNVs/exosomes disclosed herein, in embodiments, can take the form of solutions, suspensions, emulsion, drops, tablets, pills, pellets, capsules, capsules containing liquids, powders, sustained-release formulations, emulsions, aerosols, sprays, suspensions, or any other form suitable for use.

Pharmaceutical compositions comprising the therapeutic BioNVs/exosomes described herein may conveniently be presented in unit dosage forms and may be prepared by any of the methods well known in the art of pharmacy. Such methods generally include the step of bringing therapeutic agents into association with a carrier, which constitutes one or more accessory ingredients. Typically, the pharmaceutical compositions are prepared by uniformly and intimately bringing therapeutic agent into association with a liquid carrier, a finely divided solid carrier, or both, and then, if necessary, shaping the product into dosage forms of the desired formulation (e.g., wet or dry granulation, powder blends, etc., followed by tableting using conventional methods known in the art).

In embodiments, any therapeutic BioNVs/exosomes disclosed herein are formulated in accordance with routine procedures as a pharmaceutical composition adapted for a mode of administration disclosed herein.

Subjects and/or Animals

In embodiments, the subject and/or animal intended for use with therapeutic BioNVs/exosomes is a mammal, e.g., a human, mouse, rat, guinea pig, dog, cat, horse, cow, pig, rabbit, sheep, or non-human primate. In embodiments, the subject and/or animal is a non-mammal, for example, a zebrafish. In embodiments, the subject and/or animal is a transgenic animal comprising a fluorescent cell, such as, for example, an RPE cell and/or an immune cell with GFP. In embodiments, the subject and/or animal is a human. In embodiments, the therapeutic BioNVs/exosomes originate from fluorescently-tagged cells and/or are packaged with fluorescently-tagged proteins or tags (with e.g., GFP). In embodiments, the human is a pediatric human, human adult, geriatric human, an infant or child. In other embodiments, the human is referred to as a patient.

In embodiments, the method of treatment includes administering to a human who has an age in a range of from about 0 months to about 6 months old, from about 6 months to about 12 months old, from about 12 months to about 18 months old, from about 18 months to about 36 months old, from about 1 year to about 5 years old, from about 5 years to about 10 years old, from about 10 years to about 15 years old, from about 15 years to about 20 years old, from about 20 years to about 25 years old, from about 25 years to about 30 years old, from about 30 years to about 35 years old, from about 35 years to about 40 years old, from about 40 years to about 45 years old, from about 45 years to about 50 years old, from about 50 years to about 55 years old, from about 55 years to about 60 years old, from about 60 years to about 65 years old, from about 65 years to about 70 years old, from about 70 years to about 75 years old, from about 75 years to about 80 years old, from about 80 years to about 85 years old, from about 85 years to about 90 years old, from about 90 years to about 95 years old or from about 95 years to about 100 years old.

In embodiments, the subject is a non-human animal, and therefore the disclosure pertains to veterinary use. In embodiments, the non-human animal is a household pet. In embodiments, the non-human animal is a livestock animal.

In embodiments, sera and/or immune cells and/or tumor cells are evaluated and/or effected. In embodiments, immune cells include cells of a subject's and/or animal's innate immune system. In embodiments, such cells include, but are not limited to NK cell, monocyte, DC, B cell, macrophage, CD4+ T cell, and CD8+ T cell. In various embodiments, the disclosure provides for detecting a presence, detecting an absence, or measuring an amount of tumor volume, tumor cells, metastasis, cDNA, or RNA in a sample originating from a subject.

### Kits

The disclosure, in embodiments, provides kits that can simplify the administration of any agent described herein. An exemplary kit of the disclosure comprises any agent described herein in unit dosage form. In embodiments, the unit dosage form is a container, such as a pre-filled syringe, which can be sterile, containing any agent described herein and a pharmaceutically acceptable carrier, diluent, excipient, or vehicle. In embodiments, the kit further comprises a label or printed instructions instructing the use of any agent described herein. In embodiments, the kit also includes a lid speculum, topical anesthetic, and a cleaning agent for the injection surface. In embodiments, the kit further comprises one or more additional agents described herein.

In aspects, the present disclosure includes a syringe comprising one or more compositions of the present disclosure. In embodiments, the syringe is prefilled with a volume of the composition. In embodiments, the syringe is prefilled in a volume of about 1 mL to about 10 mL. In embodiments, the syringe is prefilled in a volume of about 10 mL, about 9 mL, about 8 mL, about 7 mL, about 6 mL, about 5 mL, about 4 mL, about 3 mL, about 2 mL, about 1.9 mL, about 1.8 mL, about 1.7 mL, about 1.6 mL, about 1.5 mL, about 1.4 mL, about 1.3 mL, about 1.2 mL, about 1.1 mL, or about 1.0 mL or less of the composition.

In embodiments, the syringe comprises a composition having a shelf stability ranging from about 1 hour to about 1 week. In embodiments, the syringe comprises a composition having a shelf stability of at least about 12 hours, about 24 hours, about 36 hours, about 48 hours, or about 72 hours when stored at a temperature ranging from about −85° C. to about 25° C. In embodiments, the syringe comprises a composition having a shelf stability of at least about 12 hours, about 24 hours, about 36 hours, about 48 hours, or about 72 hours when stored at a temperature ranging from about 15° C. to about 25° C.

In embodiments, the storage temperature is about −80° C. In embodiments, the storage temperature is about −20° C. In embodiments, the storage temperature is about 4° C. In embodiments, the storage temperature is about 21° C. In embodiments, the kit includes lyophilized BioNVs/exosomes.

In one embodiment, the kit comprises a container containing a composition comprising BioNVs/exosomes of the present disclosure, a therapeutically effective amount of an additional therapeutic agent, such those described herein, and instructions for use.

### EXAMPLES

### Example 1: Controlled Expression of Therapeutically-Relevant Biomolecules in Cells for Lumen-Localized Payloads in BioNVs and Exosomes

Isolated CAR NK cells will be grown to a desired log phase consistency (defined by the amount of BioNV to be derived and/or the size limits associated with growth medium flasks, vessels, or bioreactors). The cells will then be stimulated for activation. Several activation mechanisms can be used, including i) irradiated antigen presenting cells, ii) anti-CD3/CD28-coated beads, iii) IL-2, iv) a stimulatory molecule(s), such as an antigen-coated beads that are recognized by the CAR construct, v) chemicals such as phorbol esters, or vi) via other methods known to those skilled in the art.

The cells can be cleaned or separated from the stimulant for activation, such as removal from the anti-CD3/CD28-coated beads (or beads with an alternate activating antigen bound to them) by filtration, centrifugation, affinity column, and/or magnetism (e.g., if using magnetic beads). The cells may alternately be cleaned of antigen presenting cells via flow cytometry or affinity column chromatography, or combinations or both.

The cells will next be allowed to return to a resting state. Cells will be observed for their size over a period of 1-14 days to identify the resting state. After cells have returned to a resting state, a second activation process will be implemented. During this period, cells will be tested for one or more of CAR density, CD57/CD16 expression, and intracellular therapeutic protein concentration using analytic methods of enzyme-linked immunosorbent assay (ELISA), flow cytometry, iodixanol density gradient centrifugation, immunoblot, and/or PCR analysis. The cells will be restimulated by either irradiated antigen presenting cells, anti-CD3 and/or anti-CD28-coated beads (or beads with alternate activating antigen bound to them), chemicals such as phorbol esters, or other methods described in the art. This restimulation step reduces activation-induced cell death in NK cells.

The beads will then be separated, and the cells expanded as per known protocols outlined, e.g., as described in WO2020172328, WO2017037083, WO2011080740, and WO2014028453, each of which is hereby incorporated by reference in its entirety. The cell expansion can range from a 1-fold expansion and higher to the desired fold increase depending on the quantities of cells needed to derive the appropriate number of BioNVs.

The final, post-dual activated cell population will again be sampled and tested for one or more of CAR density, CD57/CD16 expression, and intracellular therapeutic protein concentration using combined analytic methods of enzyme-linked immunosorbent assay (ELISA), flow cytometry, immunoblot and/or PCR analysis.

The protocol will be conducted with the proper controls, which will vary depending on the CAR construct and purpose of the CAR-containing BioNV. One control will include cells treated in parallel and using the protocol above, that have not been activated—i.e., non-activated CAR NKs. Similar activation protocols can be used among other cell types with cell marker validation (in this case CD57/CD16 for NK cells) varying among the cell type used.

### Example 2: Generating BioNVs Via Serial Extrusion

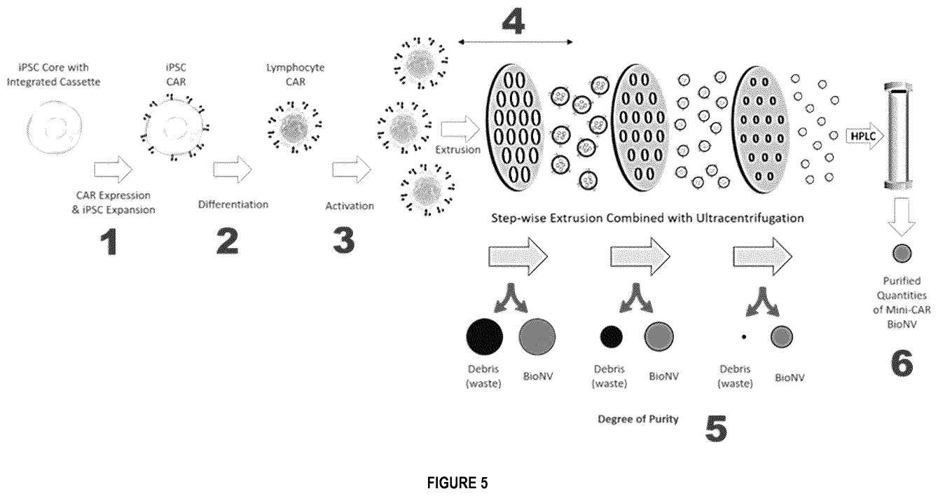

Biomimetic Nanovesicles (BioNVs) can be produced substantially as illustrated in the scheme depicted in FIG. 5.

The level of CAR expression can be measured in the hypoimmunogenic cell line using a combination of flow cytometry and iodixanol density gradient (e.g., STEP 1 of FIG. 5).

The differentiation of the iPSC-expressing surface CAR into CAR-lymphocytes can be analyzed by lymphocyte marker identification including, for example CD4/CD8 (T-cells) or CD56/CD16 (Natural Killers cells), among other cell surface markers (e.g., STEP 2 of FIG. 5). The expression profile can be determined via flow cytometry, RT-PCR, and/or CRISPR-based analytics.

Next, the activation of the CAR lymphocytes can be achieved using biomarker antigen-coated beads in low, pre-determined concentrations over the course of two weeks in two stages (e.g., STEP 3 of FIG. 5). This process can also analyze the quality of the Immunological Synapse (IS) between the CAR and the antigen-coated beads, using well-established protocols to measure i) the quantification of F-Actin accumulation at the site of synapse formation, ii) the distribution of pZeta at synapse, iii) the clustering of an antigen through the IS location, and/or iv) the polarization of lytic granules (LGs) that contain perforin and granzymes.

After the activation of the lymphocytes, the cells are expanded using established protocols (e.g., STEP 4 of FIG. 5). After expansion, the levels of perforin and granzyme (or other lumen payloads if applicable) are analyzed per cell population to ensure consistent concentration levels on a per-batch basis. This is accomplished using a series of qPCR, immunoblotting, flow cytometry, and/or mass spectrometry. The expansion step may not be necessary if a large enough cell population from Step 3 can be achieved.

Once the cells are activated to produce the desired therapeutic protein(s), they are expanded, harvested, washed several times, and then placed into a buffered extrusion medium. The cells are then wholly processed via serial extrusion through each step of the polycarbonate filter system that consists of diminishing pore size (e.g., STEP 5 of FIG. 5). In the initial extrusion step of the serial extrusion process, the nucleus (along with nuclear components including nuclear pores, genomic material, and transcription factors) and mitochondria are eliminated. The sample is then treated with endonuclease, e.g., BENZONASE. BENZONASE is a non-specific, recombinant endonuclease that cleaves all types of DNA and RNA variants into non-functional fragments <8 soluble base pairs. This leads to the highest reduction of nucleic acid load on a per sample and scalable basis and does not interfere with BioNV membrane chemistry. The cleavage process also eliminates nucleic viscosity, allowing for subsequent loading and passage of materials through the next set of extrusion filters.

The serial extrusion process will avoid the elimination of other organelles such as the Golgi Apparatus or the ER. The membrane system of these organelles is highly evolved to traffic vesicles (release and uptake) between folded membranes. For example, the cis and trans face of the Golgi Apparatus contain unique lipid compositions that facilitate low energy barrier absorption and release in the trafficking of vesicles. These components are relatively low in the cytoplasmic membrane. Therefore, isolating the cytoplasmic membrane for BioNV derivation is not as favorable. As the BioNVs are passed through the polycarbonate filters in the serial extrusion process, they undergo destruction and spontaneous formation based on the pore size. This process results in BioNVs containing membranes with a homogenous mixture of cytoplasm, Golgi, and ER lipid content and protein components that can considerably increase their affinity for cellular and tissue delivery uptake in comparison to BioNVs processed to eliminate these organelles. These features could translate to better and more consistent uptake of BioNVs into targeted cells at much lower doses than systems that do not incorporate these properties.

After the extrusion step, the BioNVs are passed through an α-CD3 HPLC (FPLC in scale-up) column to remove the low percent (approximately 0.05%) of inverted BioNVs that spontaneously form during the serial extrusion process (e.g., STEP 6 of FIG. 5). This is done to ensure the resultant BioNVs have homogenous directionality with respect to the membranes. Low loss of yield occurs during this step, as it is a flow-through process to capture impurities. Once the BioNVs have been collected after the HPLC/FPLC step, they are tested through a standardization process.

The standardization process includes one or more the following assays:

BioNV homogeneity: the use of Nanoparticle Flow Cytometry (NanoFCM) can confirm BioNV concentration, homogeneity of size, the density of the BioNVs, and/or the homogeneity of the BioNV lumen constituents.

Concentration of lumen payload: NanoFM technology can be used to determine the type and concentration of the nucleic acids/proteins that are packaged into the lumen of the BioNVs. These data can be confirmed in parallel with one or more methods including immunoblot, mass spectrometry, and BCA analyses to determine the nucleic acid and protein content of BioNVs.

BioNV Stability: one or more of nanoparticle tracking analysis (NTA), dynamic light scattering (DLS), and electron microscopy (EM) can be used in combination with immunoblot and/or mass spectrometry analyses to determine the physical and biochemical features of the BioNVs over 8 to 10 months. Data from these assays can include the protein expression profile, the degree of intact BioNV membranes/packaging, and/or the degree of aggregation.

Membrane Integrity: the integrity of the BioNV membranes is evaluated using calcein release assays combined with NanoFCM to assess membrane permeability. The results can provide insight into the leakage properties of the BioNVs against standardized BioNV panels.

Quality of lumen payload: the quality of the lumen-packaged payloads can be determined using multiple analytic assays, depending on the nature of the payload. In instances where the deliverable is a nucleic acid, qPCR and/or sequencing over 8 to 10 months can be used to check the integrity and quantity of the nucleic acid payloads. For proteins, an analysis of the BioNV constituents using one or more of NanoFCM, mass spectrometry, and immunoblot analysis can be used to analyze the protein payload.

CAR Quality and Surface Density: CAR surface density can be determined using NanoFCM, mass spectrometry, and/or immunoblot analyses. CAR surface density is expected to be at least about 5-fold to at least about 10-fold higher in BioNVs compared to whole cell surface densities. This could considerably enhance targeting to the antigen in comparison to a whole cell. CAR quality can be determined at the cellular stage as described above (e.g., as in STEP 3). A mathematical model can be used to extrapolate cellular quality data and apply it to the BioNVs in relation to efficacy study data outcomes.

BioNV Functionality: BioNVs can be tested for basic functionality, including multiple and defined standardization assays, such as in vitro cellular uptake into targeted cells with and without expressed antigen, as well their ability to cross dense tissues such as those in human retinal models. Following these basic functionality assays, which can be performed immediately after the serial extrusion process, the pre-clinical studies will address the remainder of the quality and functionality properties of the BioNVs.

### Definitions

The following definitions are used in connection with the disclosure herein. Unless defined otherwise, all technical and scientific terms used herein have the same meaning as commonly understood to one of skill in the art to which this disclosure belongs.

An “effective amount,” or “therapeutically effective amount,” is an amount that is effective for treating, preventing, or ameliorating a mammalian disease.

As used herein, “a,” “an,” or “the” can mean one or more than one.

As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present technology that do not contain those elements or features.

Although the open-ended term “comprising,” as a synonym of terms such as including, containing, or having, is used herein to describe and claim the disclosure, the present disclosure, or embodiments thereof, and may alternatively be described using alternative terms such as “consisting of” or “consisting essentially of.”

In embodiments, a “BioNV,” “exosome,” “therapeutic BioNV,” or “therapeutic exosome,” refers to biomimetic nanovesicles (NVs) which encapsulate an aqueous compartment housing at least one therapeutic biomolecule. In embodiments, therapeutic BioNVs/exosomes are allogeneic and/or hypoimmune. In embodiments, therapeutic BioNVs/exosomes comprise at least one surface-oriented, membrane-embedded CARs. In embodiments, “nanovesicles (NVs),” as referred to herein, are lipid-bound vesicles on the order of about 10 nm to about 1200 nm in size which encapsulate an aqueous core. In embodiments, lipid bound NVs can form using lipid monolayers, lipid bilayers, or maintain multilamellar forms. In embodiments, therapeutic BioNV/exosome refers to biologically derived nano-sized vesicles that can have designed biological functionalization. In embodiments, therapeutic BioNVs/exosomes are “biomimetic” in that they are derived from endogenous cellular material, more specifically, they substantially recapitulate plasma membrane material found in cells. In embodiments, the cells from which therapeutic BioNVs/exosomes originate can include stem cells of any kind, including cell types differentiated from said stem cells. In embodiments, therapeutic BioNVs/exosomes are substantially free of encapsulated cellular debris including nucleic acid, organelles, or organelle parts. In embodiments, therapeutic BioNVs/exosomes are characterized as having one or more, 2 or more, 3 or more, 4 or more, 5 or more, or 6 or more of the following:
a. being about 10 nm to about 1200 nm in size;
b. having a total volume of about 500 nm³ to about 5 μm³ (assuming spherical shape);
c. having a content of at least one phospholipid and cholesterol;
d. having a surface membrane having one or more of CD34, CCL21, PD-L1 (in BioNVs/exosomes derived from non-activated cell sources), FasL, SerpinB9, H2-M3, CD47, CTLA-4, CD24, CD200, MFG-E8, NCAM, α-phagocytic integrin, and/or anti-6R antibody or antibody format, or a chimera of any one or more thereof; having a surface membrane substantially lacking T cell receptor components (TRAC and/or TRBC), MHC class I components, and/or MHC class II components, lacking proteins of one or more of HLA-A, HLA-B, HLA-C, HLA-E or HLA-G (but not both of HLA-E and HLA-G), HLA-F, and/or CIITA, SerpinB9, substantially lacking proteins inside the vesicle of one or more of IL-4, IL-6, IL-10, and/or IL-16;
e. encapsulating one or more therapeutically relevant biomolecules, including for example, a cytokine including chemokines, interferons (IFNα/β/γ), interleukins, alarmins, lymphokines, tumor necrosis factors (TNFs), colony-stimulating factors, bone morphogenic protein (BMP), erythropoietin (EPO), granulocyte-stimulating factor (G-CSF), granulocyte macrophage colony-stimulating factor (GM-CSF), pro-inflammatory cytokines, anti-inflammatory cytokines, perforin granzyme (e.g., granzyme A, B, H, K, and M), gene editing payloads, fusion proteins, antibodies or antibody format constructs, or a combination thereof.
f. having a membrane-embedded targeting agent comprising a target-binding moiety which can include an antibody or antibody format selected from one or more of a CAR, monoclonal antibody, polyclonal antibody, antibody fragment, Fab, Fab′, Fab′-SH, F(ab′)2, Fv, single chain Fv (scFv), diabody, nanobody, linear antibody, bispecific antibody, multi-specific antibody, chimeric antibody, humanized antibody, human antibody, fusion protein comprising the antigen-binding portion of an antibody, Bispecific T cell Engagers (BiTE), viral epitope recognition receptor (VERR) or viral ligand, a variable heavy chain IgG fragment V_{H}H or V_{NAR} or through a T-Cell Receptor (TCR), wherein the targeting agent can target a single biomarker or multiple biomarkers, or multiple parts of a single biomarker;
g. being capable of adsorbing and/or encapsulating a payload of one or more perforins, granzymes, cytokines, cytotoxic proteins, non-naturally occurring cellular agents, checkpoint inhibiting agents, recombinant gene editing payloads, antibodies or antibody fragments, small molecule inhibitors, biologics, radionuclides, tracing agents, dyes, fluorescent proteins, among other therapeutic payloads, and/or any combination thereof; and
h. being capable of not causing a deleterious immune reaction in subjects.

In embodiments, “induced pluripotent stem cells,” or “iPSCs” refers to stem cells that can be generated directly from adult cells. iPSCs can originate from differentiated cells that are reprogrammed back into an embryonic-like pluripotent state. iPSCs can generally propagate indefinitely and become any cell type of the organism they originate.

In embodiments, “allogeneic,” as used herein, refers to biological material, tissues, or cells, which are genetically dissimilar and originally immunological incompatible, despite originating from the same species. Allogeneic BioNVs/exosomes, for example, are material that originates from a first subject (iPSC donor) and can be provided to any number of distinct subjects who are not genetically identical.

In embodiments, “hypoimmunogenic” or “hypoimmune,” as used herein in reference to a modified cell and/or BioNV/exosome, refers to a reduced capacity to generate an immunological response. In embodiments, iPSCs and BioNV/exosomes can be hypoimmunogenic due to reduced or ablated expression and/or activity of one or more specific cell surface proteins and/or secreted proteins, such as T cell receptor (TCR) proteins, cytokine response syndrome proteins, MHC class I or II proteins, etc. In embodiments, iPSCs and BioNVs/exosomes can be hypoimmunogenic due to increased immunoprotective cell surface proteins, such as CD47, CD34, CD24, CD200, α-phagocytic integrins, etc. In embodiments, BioNV/exosomes can be hypoimmunogenic due to not triggering CRS in a subject and/or not inducing HLA incompatibility.

In embodiments, “knocking-out,” “silencing,” “inactivating,” “disrupting,” or “blocking,” and their equivalencies, with respect to transcription, gene expression, or protein expression, refers to an amount of transcription, gene or protein expression which is reduced from a normal state or less than the wild-type state in a particular cell subset. The reduction can be significant so that no gene expression occurs, or a negligible amount of expression occurs.

In embodiments, “overexpression,” as used herein, refers to an amount of transcription, gene or protein expression which is increased from a normal state or more than the wild-type state in a particular cell subset.

Those skilled in the art will recognize, or be able to ascertain, using no more than routine experimentation, numerous equivalents to the specific embodiments described specifically herein. Such equivalents are intended to be encompassed in the scope of the following claims.

As used herein, all headings are simply for organization and are not intended to limit the disclosure in any manner. The content of any individual section may be equally applicable to all sections.

All patents and publications referenced herein are hereby incorporated by reference in their entireties, including published PCT application, WO 2020/227369, filed May 6, 2020, titled “Tailored Hypoimmune Nanovesicle Delivery Systems for Cancer Tumors,” and published U.S. non-provisional application, US 20220040106 A1, filed Aug. 3, 2021, titled “Tailored Hypoimmune Nanovesicular Delivery Systems for Cancer Tumors, Hereditary and Infectious Diseases.”

What is claimed is:

1. A method of generating a therapeutic biomimetic nanovesicle (BioNV) comprising:
(a) obtaining a hypoimmunogenic cell;
(b) activating the hypoimmunogenic cell to express one or more therapeutically relevant biomolecules; and
(c) processing the activated, hypoimmunogenic cell to generate the therapeutic BioNV, wherein the therapeutic BioNV comprises the hypoimmunogenic cell plasma membrane and encapsulates the one or more therapeutically relevant biomolecules.

2. A method of generating therapeutic exosomes comprising:
(a) obtaining a hypoimmunogenic cell;
(b) activating the hypoimmunogenic cell to express one or more therapeutically relevant biomolecules; and
(c) harvesting naturally shed therapeutic exosomes from said activated, hypoimmunogenic cell, wherein the therapeutic exosomes comprise the hypoimmunogenic cell plasma membrane and encapsulates the one or more therapeutically relevant biomolecules.

3. A method of treating or preventing a disease or disorder comprising administering a therapeutically effective amount of a therapeutic biomimetic nanovesicle (BioNV) generated by claim 1 to a subject in need thereof.

4. A method of treating or preventing a disease or disorder comprising administering a therapeutically effective amount of a therapeutic exosome generated by claim 2 in a subject in need thereof.

5. The method of any one of claims 1-4, wherein the hypoimmunogenic cell is a stem cell, an induced pluripotent stem cell (iPSC), a reprogrammed pluripotent or multipotent cell, an embryonic stem cell, a mesenchymal stem cell, or a differentiated cell which originates from any stem cell thereof.

6. The method of any one of claims 1-4, wherein the hypoimmunogenic cell is a T cell, helper T cell, T-memory cell, or NK cell.

7. The method of any one of claims 1-4, wherein the hypoimmunogenic cell is a macrophage.

8. The method of any one of claims 1-4, wherein the hypoimmunogenic cell is a monocyte.

9. The method of any one of claims 1-4, wherein the hypoimmunogenic cell is a hepatocyte, a cardiomyocyte, a neuron, an endothelial cell, a pancreatic cell, or a retinal pigmented epithelium (RPE) cell.

10. The method of any one of the preceding claims, wherein the hypoimmunogenic cell substantially lacks one or more MHC class I proteins, MHC class II proteins, T cell receptor (TCR) proteins, and/or cytokine release syndrome (CRS) proteins.

11. The method of any one of the preceding claims, wherein the hypoimmunogenic cell has a β2-macroglobulin (B2M) gene disruption and/or a disruption that reduces or ablates MHC class I protein expression and/or activity.

12. The method of any one of the preceding claims, wherein the hypoimmunogenic cell has a CIITA gene disruption and/or a disruption that reduces or ablates MHC class II protein expression and/or activity.

13. The method of any one of the preceding claims, wherein the hypoimmunogenic cell has an HLA-A gene disruption and/or a disruption that reduces or ablates HLA-A protein expression and/or activity.

14. The method of any one of the preceding claims, wherein the hypoimmunogenic cell has an HLA-B gene disruption and/or a disruption that reduces or ablates HLA-B protein expression and/or activity.

15. The method of any one of the preceding claims, wherein the hypoimmunogenic cell has an HLA-C gene disruption and/or a disruption that reduces or ablates HLA-C protein expression and/or activity.

16. The method of any one of the preceding claims, wherein the hypoimmunogenic cell has an HLA-E gene disruption or an HLA-G gene disruption and/or a disruption that reduces or ablates HLA-E or HLA-G protein expression and/or activity.

17. The method of any one of the preceding claims, wherein the hypoimmunogenic cell has an HLA-F gene disruption and/or a disruption that reduces or ablates HLA-F protein expression and/or activity.

18. The method of any one of the preceding claims, wherein the hypoimmunogenic cell has a T cell alpha constant (TRAC) gene disruption and/or a disruption that reduces or ablates TRAC protein expression and/or activity.

19. The method of any one of the preceding claims, wherein the hypoimmunogenic cell has a T cell beta constant (TRBC) gene disruption and/or a disruption that reduces or ablates TRBC protein expression and/or activity.

20. The method of any one of the preceding claims, wherein the hypoimmunogenic cell has reduced or ablated expression of a PD-1 gene and/or reduced or ablated PD-1 protein expression and/or activity, wherein the hypoimmunogenic cell is activated; or the hypoimmunogenic cell has expression or increased expression of a PD-1 gene and/or gene product, wherein the hypoimmunogenic cell is not activated.

21. The method of any one of the preceding claims, wherein the hypoimmunogenic cell has an IL-4 gene disruption and/or a disruption that reduces or ablates IL-4 protein expression and/or activity.

22. The method of any one of the preceding claims, wherein the hypoimmunogenic cell has an IL-6 gene disruption and/or a disruption that reduces or ablates IL-6 protein expression and/or activity.

23. The method of any one of the preceding claims, wherein the hypoimmunogenic cell has an IL-10 gene disruption and/or a disruption that reduces or ablates IL-10 protein expression and/or activity.

24. The method of any one of the preceding claims, wherein the hypoimmunogenic cell has an IL-16 gene disruption and/or a disruption that reduces or ablates IL-16 protein expression and/or activity.

25. The method of any one of the preceding claims, wherein the hypoimmunogenic cell has a SerpinB9 gene disruption and/or a disruption that reduces or ablates SerpinB9 protein expression and/or activity.

26. The method of any one of claims 1-24, wherein the hypoimmunogenic cell expresses or has increased expression and/or activity of a SerpinB9 gene and/or gene product.

27. The method of any one of the preceding claims, wherein the hypoimmunogenic cell expresses or has increased expression and/or activity of a CCL2 gene and/or gene product.

28. The method of any one of the preceding claims, wherein the hypoimmunogenic cell expresses or has increased expression of a PD-L1 gene and/or gene product, and wherein the hypoimmunogenic cell is not activated; or wherein the modified cell has reduced or ablated expression of a PD-L1 gene and/or gene product, and wherein the hypoimmunogenic cell is activated.

29. The method of any one of the preceding claims, wherein the hypoimmunogenic cell expresses or has increased expression and/or activity of a H2-M3 gene and/or gene product.

30. The method of any one of the preceding claims, wherein the hypoimmunogenic cell expresses or has increased expression and/or activity of a CD47 gene and/or gene product.

31. The method of any one of the preceding claims, wherein the hypoimmunogenic cell expresses or has increased expression and/or activity of a CD24 gene and/or gene product.

32. The method of any one of the preceding claims, wherein the hypoimmunogenic cell expresses or has increased expression and/or activity of a chimeric CD24/CD47 gene and/or gene product.

33. The method of any one of the preceding claims, wherein the hypoimmunogenic cell expresses or has increased expression and/or activity of a CTLA-4 gene and/or gene product.

34. The method of any one of the preceding claims, wherein the hypoimmunogenic cell expresses or has increased expression and/or activity of a CD200 gene and/or gene product.

35. The method of any one of the preceding claims, wherein the hypoimmunogenic cell expresses or has increased expression and/or activity of a chimeric CD24/CD200 gene and/or gene product or a chimeric CD47/CD200 gene and/or gene product.

36. The method of any one of the preceding claims, wherein the hypoimmunogenic cell expresses or has increased expression and/or activity of an MFG-E8 gene and/or gene product.

37. The method of any one of the preceding claims, wherein the hypoimmunogenic cell expresses or has increased expression and/or activity of a NCAM gene and/or gene product.

38. The method of any one of the preceding claims, wherein the hypoimmunogenic cell expresses or has increased expression and/or activity of an α-phagocytic integrin gene and/or gene product.

39. The method of any one of the preceding claims, wherein the hypoimmunogenic cell expresses or has increased expression and/or activity of an antibody or antibody format molecule which targets an IL-6 surface receptor (anti-IL-6R).

40. The method of any one of the preceding claims, wherein the hypoimmunogenic cell expresses a FasL gene and/or gene product.

41. The method of any one of the preceding claims, wherein the hypoimmunogenic cell does not overexpress a FasL gene and/or gene product.

42. The method of any one of the preceding claims, wherein the hypoimmunogenic cell substantially lacks expression and/or activity of one or more immunogenic proteins and expresses or has increased expression of one or more immunoprotective proteins.

43. The method of any one of the preceding claims, wherein the hypoimmunogenic cell has reduced or ablated expression and/or activity of 3 or more immunogenic proteins, 4 or more immunogenic proteins, 5 or more immunogenic proteins, 6 or more immunogenic proteins, 7 or more immunogenic proteins, 8 or more immunogenic proteins, 9 or more immunogenic proteins, 10 or more immunogenic proteins, 11 or more immunogenic proteins, or 12 or more immunogenic proteins.

44. The method of any one of the preceding claims, wherein the hypoimmunogenic cell expresses or has increased expression of 3 or more immunoprotective proteins, 4 or more immunoprotective proteins, 5 or more immunoprotective proteins, 6 or more immunoprotective proteins, 7 or more immunoprotective proteins, 8 or more immunoprotective proteins, 9 or more immunoprotective proteins, or 10 or more immunoprotective proteins.

45. The method of any one of the preceding claims, wherein the hypoimmunogenic cell has reduced or ablated expression and/or activity of a gene and/or gene product of HLA-A, HLA-B, HLA-C, HLA-F, CIITA, IL-6, TRAC, TRBC, and one of either HLA-E or HLA-G.

46. The method of any one of claims 1-44, wherein the hypoimmunogenic cell has reduced or ablated expression and/or activity of a gene and/or gene product of HLA-A, HLA-B, HLA-C, HLA-F, CIITA, IL-6, TRAC, TRBC, SerpinB9, and one of either HLA-E or HLA-G.

47. The method of any one of claims 1-44, wherein the hypoimmunogenic cell comprises a hypoimmunogenic cell that has reduced or ablated expression and/or activity of a gene and/or gene product of HLA-A, HLA-B, HLA-C, HLA-F, CIITA, IL-6, TRAC, TRBC, SerpinB9, one of either HLA-E or HLA-G, and one or more of IL-4, IL-10, and IL-16.

48. The method of any one of claims 1-47, wherein the hypoimmunogenic cell expresses or has increased expression and/or activity of α-phagocytic integrin, CCL2, H2-M3, FasL, MFEG8, and PD-L1 and/or CTLA-4, wherein the hypoimmunogenic cell does not overexpress FasL, and wherein the hypoimmunogenic cell is not activated with the expression of PD-L1; and
expresses or has increased expression and/or activity of one of either CD24, CD47, CD200, chimeric CD24/CD47, chimeric CD24/CD200, and chimeric CD47/CD200, or two of either CD24, CD47, and CD200.

49. The method of any one of claims 1-47, wherein the hypoimmunogenic cell expresses or has increased expression and/or activity of α-phagocytic integrin, CCL2, H2-M3, FasL, MFEG8, SerpinB9, and PD-L1 and/or CTLA-4, wherein the hypoimmunogenic cell does not overexpress FasL, and wherein the hypoimmunogenic cell is not activated with the expression of PD-L1; and
expresses or has increased expression and/or activity of one of either CD24, CD47, CD200, chimeric CD24/CD47, chimeric CD24/CD200, and chimeric CD47/CD200, or two of either CD24, CD47, and CD200.

50. The method of any one of claims 1-47, wherein the hypoimmunogenic cell expresses or has increased expression and/or activity of a CD200 gene and/or gene product and does not express and/or substantially lacks either a CD24 or a CD47 gene and/or gene product.

51. The method of any one of claims 1-47, wherein the hypoimmunogenic cell has no expression and/or activity of a SerpinB9 gene and/or gene product and a CD200 gene and/or gene product.

52. The method of any one of the preceding claims, wherein the hypoimmunogenic cell is allogeneic.

53. The method of any one of the preceding claims, wherein the hypoimmunogenic cell does not cause an immune reaction in patients to which it or a BioNV or exosome derived therefrom is administered.

54. The method of any one of the preceding claims, wherein the hypoimmunogenic cell comprises one or more targeting agents.

55. The method of claim 54, wherein the one or more targeting agents comprises a chimeric antigen receptor (CAR).

56. The method of claim 55, wherein the CAR is bispecific.

57. The method of claim 55, wherein the CAR lacks an intracellular portion.

58. The method of claim 55, wherein the CAR comprises a targeting agent, a transmembrane domain, and an intracellular domain, which comprises a costimulatory domain and/or a signaling domain.

59. The method of claim 58, wherein the transmembrane domain is derived from CD28, CD3ζ, CD4, CD8a, or ICOS, or a fragment thereof.

60. The method of claim 58, wherein the intracellular domain comprises an intracellular signaling domain of a CD3ζ-chain and/or one or more co-stimulatory molecules, optionally selected from CD28, 4-1BB, ICOS, CD27, and OX40.

61. The method of claim 54, wherein the one or more targeting agents comprises an antibody or antibody format.

62. The method of claim 61, wherein the antibody or antibody format is selected from one or more of a monoclonal antibody, polyclonal antibody, antibody fragment, Fab, Fab′, Fab′-SH, F(ab′)2, Fv, single chain Fv (scFv), V_{NAR}, V_{H}H, afflilin, diabody, nanobody, linear antibody, bispecific antibody, multi-specific antibody, chimeric antibody, humanized antibody, human antibody, and fusion protein comprising the antigen-binding portion of an antibody.

63. The method of claim 62, wherein the antibody format is a scFv.

64. The method of claim 54, wherein the one or more targeting agents comprises a viral epitope recognition receptor (VERR) and/or viral ligand.

65. The method of claim 54, wherein the one or more targeting agents comprises a ligand for a receptor and/or a receptor for a ligand.

66. The method of anyone of the preceding claims, wherein activating the hypoimmunogenic cell comprises activation of a TCR/CD3 receptor complex by a protein antigen.

67. The method of any one of claims 1-65, wherein activating the hypoimmunogenic cell comprises activation of a TCR/CD3 receptor complex by a small molecule.

68. The method of any one of claims 1-65, wherein activating the hypoimmunogenic cell comprises activation of a TCR/CD3 receptor complex by a viral antigen.

69. The method of any one of claims 1-65, wherein activating the hypoimmunogenic cell comprises activation of a calcium dependent channel.

70. The method of any one of claims 1-65, wherein activating the hypoimmunogenic cell comprises activation of an LFA-1 integrin receptor.

71. The method of any one of claims 1-65, wherein activating the hypoimmunogenic cell comprises activation of a CD28 receptor.

72. The method of any one of claims 1-65, wherein activating the hypoimmunogenic cell comprises activation of an IL-2 receptor.

73. The method of any one of claims 1-65, wherein activating the hypoimmunogenic cell comprises activation of a SLAMF1(CD150) receptor.

74. The method of claim 73, wherein the activation of the SLAMF1(CD150) receptor is by a measles virus.

75. The method of claim 73, wherein the activation of the SLAMF1(CD150) receptor is by a Gram-negative bacteria.

76. The method of any one of claims 1-65, wherein activating the hypoimmunogenic cell comprises activation of an IFNγ receptor.

77. The method of any one of claims 1-65, wherein activating the hypoimmunogenic cell comprises activation of a CD4 receptor by one or more viruses.

78. The method of any one of claims 1-65, wherein activating the hypoimmunogenic cell comprises activation by an engineered, non-native biomolecule selected from one or more of a soluble peptide, a chimeric antigen receptor, a small molecule decoy, a small molecule ligand, a designer nucleic acid ligand, a carbohydrate ligand, a viral ligand, a chimeric biomolecular ligand, a fusion protein, an antibody, and an antibody format molecule.

79. The method of any one of claims 1-65, wherein activating the hypoimmunogenic cell comprises activation by an inorganic compound.

80. The method of any one of claims 1-65, wherein activating the hypoimmunogenic cell comprises activation by expression, overexpression, or increased activity of a transcription factor.

81. The method of any one of the preceding claims, wherein activating the hypoimmunogenic cell comprises activation at the DNA level by one or more of a transposase-based method, Cre/Lox-based method, endonuclease-based method, homologous recombination (HR)-based method, non-homologous end joining (NEHJ)-based method, microhomology-mediated end-joining (MMEJ)-based method, homology-mediated end joining (HMEJ)-based method, small RNA, or a combination thereof.

82. The method of claim 81, wherein the small RNA comprises one or more of a guide RNA (gRNA), tracer RNA (tracrRNA), micro RNA (miRNA), RNA inference (RNAi), small interference RNA (siRNA), duplex RNA, Piwi-interacting RNA (piRNA), small nuclear RNA (snRNA), small nucleolar RNA (snoRNA), antisense oligonucleotide (ASO), locked nucleic acid (LNA), splice switching oligonucleotide (SSO), tRNA, complementary messenger RNA, repeat associated small interfering RNA (rasiRNA), endonuclease, and small non-coding RNA.

83. The method of any one claims 1-80, wherein activating the hypoimmunogenic cell comprises activation at the RNA level by one or more guide RNA (gRNA), tracer RNA (tracrRNA), micro RNA (miRNA), RNA inference (RNAi), small interference RNA (siRNA), duplex RNA, Piwi-interacting RNA (piRNA), small nuclear RNA (snRNA), small nucleolar RNA (snoRNA), antisense oligonucleotide (ASO), locked nucleic acid (LNA), splice switching oligonucleotide (SSO), tRNA, complementary messenger RNA, repeat associated small interfering RNA (rasiRNA), endonuclease, small non-coding RNA, IRES element, or a combination thereof.

84. The method of any one of claims 1-80, wherein activating comprises one or more RNA-guided endonucleases.

85. The method of any one of claims 1-80, wherein activating the hypoimmunogenic cell comprises activation by an endogenous promoter region and/or enhancer region.

86. The method of any one of claims 1-80, wherein activating the hypoimmunogenic cell comprises activation by stably integrating a genetic element.

87. The method of any one of claims 1-80, wherein activating the hypoimmunogenic cell comprises activation by transient expression of a genetic element.

88. The method of any one of the preceding claims, wherein activating the hypoimmunogenic cell results in a metabolically altered state of the hypoimmunogenic cell.

89. The method of any one of the preceding claims, wherein the one or more therapeutically relevant biomolecules is a chemokine, interferon, interleukin, alarmin, lymphokine, perforin, granzyme, granulysin, tumor necrosis factor (TNF), colony-stimulating factor, bone morphogenic protein (BMP), erythropoietin (EPO), granulocyte-stimulating factor (G-CSF), granulocyte macrophage colony-stimulating factor (GM-CSF), gene editing payload, fusion protein, antibody or antibody format, or a combination thereof.

90. The method of claim 89, wherein the cytokine is a pro-inflammatory cytokine.

91. The method of claim 89, wherein the cytokine is an anti-inflammatory cytokine.

92. The method of claim 89, wherein the granzyme is granzyme A, B, H, K, or M.

93. The method of claim 89, wherein the gene editing payload comprises one or more gene editor nucleic acids and/or proteins, or one or more nucleic acids encoding one or more gene editors.

94. The method of claim 93, wherein the one or more gene editors is a site-directed endonuclease, TALEN, ZFN, RNase P RNA, CRISPR/Cas nuclease, C2c1, C2c2, C2c3, Cas9, Cpf1, TevCas9, Archaea Cas9, CasY.1, CasY.2, CasY.3, CasY.4, CasY.5, CasY.6, CasX, Cas omega, transposase, and/or any ortholog or homolog thereof.

95. The method of claim 89, wherein the gene editing payload comprises a transactivating response region (TAR) loop system.

96. The method of any one of claims 1, 3 and 5-95, wherein the therapeutic BioNV comprises:
(i) one or more membrane-embedded targeting agents targeted against one or more cellular biomarkers;
(ii) one or more membrane-embedded proteins of an α-phagocytic integrin, CCL2, H2-M3, FasL, MFEG8, PD-L1 and/or CTLA-4, SerpinB9, and anti-IL-6R antibody or antibody format;
(iii) a membrane-embedded protein of one of either CD24, CD47, CD200, chimeric CD24/CD47, chimeric CD24/CD200, and chimeric CD47/CD200, or two of either CD24, CD47, and CD200; and
(iv) a membrane substantially lacking proteins of one or more of HLA-A, HLA-B, HLA-C, HLA-F, CIITA, IL-6, TRAC, TRBC, one of either HLA-E or HLA-G, and one or more of IL-4, IL-10, and IL-16, and
wherein the one or more therapeutically relevant biomolecules is a chemokine, interferon, interleukin, alarmin, lymphokine, perforin, tumor necrosis factor (TNF), colony-stimulating factor, bone morphogenic protein (BMP), erythropoietin (EPO), granulocyte-stimulating factor (G-CSF), granulocyte macrophage colony-stimulating factor (GM-CSF), gene editing payload, fusion protein, antibody or antibody format, or a combination thereof.

97. The method of any one of claims 1, 3 and 5-95, wherein the therapeutic BioNV comprises:
(i) one or more membrane-embedded targeting agents targeted against one or more cellular biomarkers;
(ii) one or more membrane-embedded proteins of an α-phagocytic integrin, CCL2, H2-M3, FasL, MFEG8, PD-L1 and/or CTLA-4, and anti-IL-6R antibody or antibody format;
(iii) a membrane-embedded protein of either CD24 and CD47, or a chimeric CD24/CD47; and
(iv) a membrane substantially lacking proteins of one or more of HLA-A, HLA-B, HLA-C, HLA-F, CIITA, IL-6, TRAC, TRBC, one of either HLA-E or HLA-G, SerpinB9 and CD200, and one or more of IL-4, IL-10, and IL-16, and
wherein the one or more therapeutically relevant biomolecules is a chemokine, interferon, interleukin, alarmin, lymphokine, perforin, granzyme, granulysin, tumor necrosis factor (TNF), colony-stimulating factor, bone morphogenic protein (BMP), erythropoietin (EPO), granulocyte-stimulating factor (G-CSF), granulocyte macrophage colony-stimulating factor (GM-CSF), gene editing payload, fusion protein, antibody or antibody format, or a combination thereof.

98. The method of claim 96 or 97, wherein the one or more targeting agents is an antibody or antibody format.

99. The method of claim 98, wherein the one or more targeting agents is a CAR.

100. The method of any one of claims 1 and 96-99, wherein processing the activated, hypoimmunogenic cell is by one or more of sonication, adaptive focused acoustics technology, French press, extrusion, serial extrusion, enzymatic rupture, cell lysis by detergent, and/or electroporation.

101. The method of claim 100, wherein processing the activated, hypoimmunogenic cell is by serial extrusion.

102. The method of any one of claims 1 and 96-101, wherein the therapeutic BioNV of about 10 nm to about 1200 nm in size.

103. The method of claim 102, wherein the therapeutic BioNV is about 10 nm to about 100 nm in size.

104. The method of claim 102, wherein the therapeutic BioNV is about 100 nm to about 200 nm in size.

105. The method of claim 102, wherein the therapeutic BioNV is about 200 nm to about 500 nm in size.

106. The method of claim 102, wherein the therapeutic BioNV is about 500 nm to about 1200 in size.

107. The method of any one of claims 2 and 4-95, wherein the therapeutic exosome comprises:
(i) one or more membrane-embedded targeting agents targeted against one or more cellular biomarkers;
(ii) one or more membrane-embedded proteins of an α-phagocytic integrin, CCL2, H2-M3, FasL, MFEG8, PD-L1 and/or CTLA-4, SerpinB9, and anti-IL-6R antibody or antibody format;
(iii) a membrane-embedded protein of one of either CD24, CD47, CD200, chimeric CD24/CD47, chimeric CD24/CD200, and chimeric CD47/CD200, or two of either CD24, CD47, and CD200; and
(iv) a membrane substantially lacking proteins of one or more of HLA-A, HLA-B, HLA-C, HLA-F, CIITA, IL-6, TRAC, TRBC, one of either HLA-E or HLA-G, and one or more of IL-4, IL-10, and IL-16, and
wherein the one or more therapeutically relevant biomolecules is a chemokine, interferon, interleukin, alarmin, lymphokine, perforin, tumor necrosis factor (TNF), colony-stimulating factor, bone morphogenic protein (BMP), erythropoietin (EPO), granulocyte-stimulating factor (G-CSF), granulocyte macrophage colony-stimulating factor (GM-CSF), gene editing payload, fusion protein, antibody or antibody format, or a combination thereof.

108. The method of any one of claims 2 and 4-95, wherein the therapeutic exosome comprises:
(i) one or more membrane-embedded targeting agents targeted against one or more cellular biomarkers;
(ii) one or more membrane-embedded proteins of an α-phagocytic integrin, CCL2, H2-M3, FasL, MFEG8, PD-L1 and/or CTLA-4, SerpinB9, and anti-IL-6R antibody or antibody format;
(iii) a membrane-embedded protein of either CD24 and CD47, or a chimeric CD24/CD47; and
(iv) a membrane substantially lacking proteins of one or more of HLA-A, HLA-B, HLA-C, HLA-F, CIITA, IL-6, TRAC, TRBC, one of either HLA-E or HLA-G, SerpinB9 and CD200, and one or more of IL-4, IL-10, and IL-16, and
wherein the one or more therapeutically relevant biomolecules is a chemokine, interferon, interleukin, alarmin, lymphokine, perforin, granzyme, granulysin, tumor necrosis factor (TNF), colony-stimulating factor, bone morphogenic protein (BMP), erythropoietin (EPO), granulocyte-stimulating factor (G-CSF), granulocyte macrophage colony-stimulating factor (GM-CSF), gene editing payload, fusion protein, antibody or antibody format, or a combination thereof.

109. The method of claim 107 or 108, wherein the one or more targeting agents is an antibody or antibody format.

110. The method of claim 109, wherein the one or more targeting agents is a CAR.

111. The method of any one of claims 2 and 107-110, wherein harvesting the therapeutic exosomes from the activated, hypoimmunogenic cell comprises inducing hypoxia.

112. The method of any one of claims 2 and 107-111, wherein harvesting the therapeutic exosomes from the activated, hypoimmunogenic cell comprises expressing or increasing expression of one or more cellular factors.

113. The method of any one of claims 2 and 107-112, wherein harvesting the therapeutic exosomes from the activated, hypoimmunogenic cell comprises supplying one or more small molecule exosome modulators.

114. The method of any one of claims 2 and 107-113, wherein harvesting the therapeutic exosomes from the activated, hypoimmunogenic cell comprises supplementing the media with one or more exosome factors.

115. The method of any one of claims 2 and 110-114, wherein the therapeutic exosome is about 10 nm to about 200 nm in size.

116. The method of claim 115, wherein the therapeutic exosome is about 10 nm to about 100 nm in size.

117. The method of claim 115, wherein the therapeutic exosome is about 100 nm to about 200 nm in size.

118. The method of claim 3 or 4, wherein the mammalian disease is a cancer, infectious disease, hereditary disorder, or an orphan disease.

119. The method of claim 1 or 2, wherein activating the hypoimmunogenic cell includes using INF-α to activate the JAK1/TYK2 to STAT pathway via the INFAR1 and/or INFAR2 receptors.

120. The method of any one of claims 1-2 and 119, wherein the hypoimmunogenic cell is a natural killer cell and wherein the activating comprises activating a receptor chosen from the group selected from CD2, CD3, CD16, CD314 (NKG2D), CD335, B7-H6, CD158d, IL-2R, IL-12R, DNAM-1, CD2, CD44, CD137, CX3CR1, CD27, CD160, 2B4, and combinations thereof.

121. The method of any one of claims 1-2 and 119-120, wherein the hypoimmunogenic cell is a natural killer cell, Killer T-cell, T-cell (all subsets) and wherein the activating does not lead to degranulation or granule polarization to an immunological synapse.

122. The method of any one of claims 1-2 and 119-121, wherein the hypoimmunogenic cell is a natural killer cell, Killer T-cell, T-cell (all subsets) and wherein the activating leads to granulation.

123. The method of any one of claims 1-2 and 119-122, wherein the hypoimmunogenic cell is a natural killer cell, Killer T-cell, T-cell (all subsets) and wherein the activating leads to polarization of lytic granules.

124. The method of any one of claims 1-2 and 119-123, wherein the activating comprises activating the expression of cytotoxic biomolecules by targeting cytoplasmic signaling molecules that are part of a signaling pathway.

125. The method of any one of claims 1-2 and 119-124, wherein the activating comprises activating PKC by a small molecule or biologic.

126. The method of any one of claims 1-2 and 119-125, wherein the activating comprises activating PKC by calcium and diacylglycerol.

127. The method of any one of claims 1-2 and 119-126, wherein the activating step comprises activating PKC by adding a molecule to the hypoimmunogenic cell selected from the group consisting of Bryostatin 1, Ingenol-3-angelate, Phorbol 12-myristate 13-acetate, Prostratin, SC-9, SC-10, and Phorbol-12,13-dibutyrate.

128. The method of any one of claims 1-2 and 119-120, wherein the activating comprises targeting Crk for perforin and granzyme expression with a small molecule or biologic.

129. The method of claim 128, wherein said targeting Crk step comprises blocking or mutating Crk phosphorylation.

130. The method of any one of claims 1-2 and 119-129, wherein the activating comprises activating transcription factors that lead to activating a compound chosen from the group consisting of perforins, granzymes, other cytotoxic proteins, and combinations thereof.

131. The method of any one of claims 1-2 and 119-130, wherein the activating further comprises activating or repressing gene expression function by CRISPR activation and inhibition systems (CRISPRa/i) of a compound chosen from the group consisting of perforins, granzymes, other cytotoxic proteins, and combinations thereof.

132. The method of any one of claims 1-2 and 119-131, wherein the hypoimmunogenic cell is an induced pluripotent stem cell (iPSC) engineered to comprise at least one perforin and/or granzyme gene are and wherein the iPSC comprises regulatory properties or elements embedded in one or more promoters thereof, allowing direct and controlled expression of the at least one perforin and/or granzyme gene.

133. The method of any one of claims 1-2 and 119-132, further comprising analyzing a substance of the activated hypo immunogenic cell selected from the group consisting of surface markers, cytoplasmic proteins, differences in mRNA expression profiles, differences in DNA expression profiles, and combinations thereof.

134. The method of any one of claims 1-2 and 119-133, further comprising measuring a degree of activation of the activated hypoimmunogenic cell.

135. A method for treating a disease or disorder comprising:
administering a therapeutically effective amount of a biomimetic nanovesicle (BioNV) comprising one or more BioNV antigen-binding constructs; and
administering a therapeutically effective amount of a whole cell comprising one or more whole cell antigen-binding constructs.

136. The method of claim 135, wherein the disease or disorder is cancer.

137. The method of claim 136, wherein the cancer is one or more of a carcinoma, a sarcoma, a myeloma, a leukemia, a lymphoma, a mixed-type cancer, and/or a metastatic cancer.

138. The method of any one of claims 135-137, wherein the BioNV is administered first and the whole cell is administered second, the whole cell is administered first and the BioNV is administered second, or the BioNV and the whole cell are administered contemporaneously.

139. The method of any one of claims 135-138, wherein the one or more BioNV antigen-binding constructs comprises a CAR.

140. The method of any one of claims 135-139, wherein the one or more whole cell antigen-binding comprises a CAR.

141. The method of any one of claims 135-140, wherein the one or more whole cell antigen-binding constructs binds a cancer cell.

142. The method of any one of claims 135-141, wherein the one or more BioNV antigen-binding constructs binds a cancer cell.

143. The method of any one of claims 135-142, wherein the one or more whole cell antigen-binding constructs binds the BioNV.

144. The method of any one of claims 135-143, wherein the one or more BioNV antigen-binding constructs binds the whole cell.

145. The method of any one of claims 135-144, wherein the whole cell comprises a CAR that binds an antigen, ligand, or receptor present on both a cancer cell and the BioNV.

146. The method of any one of claims 135-145, wherein the BioNV comprises:
(i) a CAR that binds an antigen, ligand, or receptor present on a cancer cell; and
(ii) an antigen, ligand, and/or receptor that binds a CAR on the whole cell.

147. The method of any one of claims 135-146, wherein the whole cell comprises:
(i) a first CAR that binds an antigen, ligand, or receptor present on a cancer cell; and
(ii) a second CAR that binds an antigen, ligand, and/or receptor present on the BioNV.

148. The method of claim 147, wherein the antigen, ligand, and/or receptor on the BioNV is, comprises, or resembles, the antigen, ligand, or receptor present on the cancer cell; or wherein the antigen, ligand, and/or receptor on the BioNV is different from the antigen, ligand, or receptor present on the cancer cell.

149. The method of claim 147 or 148, wherein the first CAR is capable of signaling via a pathway that results in cell-mediated cytotoxicity, or comprises one or more intracellular signaling domains capable of signaling via a pathway that results in cell-mediated cytotoxicity.

150. The method of claim 147 or 148, wherein the second CAR is capable of signaling via a pathway that results in cell-mediated cytotoxicity by the whole cell, or comprises one or more intracellular signaling domains capable of signaling via a pathway that results in cell-mediated cytotoxicity.

151. The method of claim 147 or 148, wherein the second CAR is not capable of signaling via a pathway that results in cell-mediated cytotoxicity by the whole cell, or lacks one or more intracellular signaling domains capable of signaling via a pathway that results in cell-mediated cytotoxicity.

152. The method of claim 147 or 148, wherein the second CAR is capable of signaling via a pathway that results in persistence, survival, and/or proliferation of the whole cell, or comprises one or more intracellular signaling domains capable of signaling via a pathway that results in persistence, survival, and/or proliferation of the whole cell.

153. The method of any one of claims 135-152, wherein administering the BioNV improves the functionality of the whole cell.

154. The method of claim 153, wherein the functionality of the whole cell comprises one or more of cell-mediated cytotoxicity, cytokine release, tumor cell or cancer cell killing, honing to a tumor cell or cancer tissue, tissue infiltration, proliferation, persistence, and/or survival.

155. The method of any one of claims 135-154, wherein administering the BioNV reduces one or more toxicities of the whole cell.
